(19) United States
(12) Patent Application Publication
(10) Pub. No.: US 2025/0213674 A1
(43) Pub. Date: Jul. 3, 2025

(54) VIRAL VECTORS

(71) Applicants: UNIVERSITY COURT OF THE UNIVERSITY OF ST ANDREWS, Fife (GB); CITY, UNIVERSITY OF LONDON, London (GB)

(72) Inventors: Richard RANDALL, St Andrews (GB); Stephen GOODBOURN, London (GB)

(21) Appl. No.: 18/881,088

(22) PCT Filed: Jul. 5, 2023
(86) PCT No.: PCT/GB2023/051766

§ 371 Date: Jan. 3, 2025

(30) Foreign Application Priority Data
Jul. 5, 2022 (GB) 2209861.0

(51) Int. Cl. A61K 39/155; A61P 37/04; C12N 7/00; C12N 15/86

(52) U.S. Cl. CPC A61K 39/155; A61P 37/04; C12N 7/00; C12N 15/86; C12N 2760/18034; C12N 2760/18043; C12N 2760/18052

(57) ABSTRACT

The present disclosure provides a cohort of novel viral genome derived products for use, for example, in medicine, as a medicaments, as expression vectors and as adjuvants. The disclosed viral genome derived products may be derived from members of the Paramyxoviridae Family which contains a wide variety of vertebrate viruses, including mumps, measles and human parainfluenza viruses.

### FIELD

The present disclosure provides viral vectors with therapeutic utility and a use in the generation and expression of recombinant proteins.

### BACKGROUND

Virus can be modified for use as vectors to deliver therapeutic compounds to cells and other systems. Vectors based on PIV5 are useful as the virus is able to infect human cells but is not known to cause any disease. Moreover, the virus possesses a non-segmented negative strand RNA genome and does not move through a DNA phase when replicating in a cell—this avoids the possibility of viral genes being integrated into the host genome.

There is a need for improved vectors which can be used as adjuvants and which can be used in medicine to treat diseases like cancer.

### SUMMARY

The present disclosure provides a cohort of novel viral genome derived products for use:
in medicine;
as a medicament;
as expression vectors;
in the delivery of therapeutic proteins;
in the production of recombinant/therapeutic proteins;
as adjuvants;
as vaccine adjuvants;
in the induction of immune responses, including, for example innate immune responses;
in the treatment and/or prevention of acute or chronic diseases and infections; and in the treatment and prevention of cancer.

The term ‘adjuvant’ may be applied to any product, molecule or compound which is able to augment or modulate an immune response to another product, molecule or compound. For example, adjuvants are often used to improve immune responses to antigens, wherein those antigens are, by themselves, poorly immunogenic or not capable of eliciting the correct immune response. An adjuvant may be administered together with the product (e.g. an antigen), molecule or compound which is to be subject to a modulated or augmented (for example improved) immune response. An adjuvant may be administered with a vaccine (as a ‘vaccine adjuvant’) to improve the immune response to the vaccine.

It should be noted that while this application refers to various viral genome derived products for use in the treatment and/or prevention of certain diseases, conditions and/or disorders, the disclosure extends to methods of treating those same diseases, conditions and/or disorders. Said methods may (generally) comprise steps in which subjects in need of treatment are administered any one or more of the viral genome derived products described herein. The subjects may be administered therapeutically effective amounts of any of the viral genome derived products described herein.

Moreover, throughout this specification, the term “comprising” is used to denote that the disclosed embodiments and teachings “comprise” the noted features and as such, may also include other features. However, the term “comprising” may also encompass embodiments and teachings which “consist essentially of” the relevant features or “consist of” the relevant features.

The disclosed viral genome derived products may be derived from members of the Paramyxoviridae Family. The Paramyxoviridae Family contains a wide variety of vertebrate viruses, including mumps, measles and human parainfluenza viruses.

In one teaching, the disclosed viral genome derived products are derived from the parainfluenza virus 5 (PIV5) genome. As such, the disclosed viral genome derived products are all PIV5 derived products.

PIV5 has a non-segmented, negative-sense RNA genome of 15,246 nucleotides (nt) containing seven tandemly arranged genes, that encode eight proteins, flanked by 3′-leader (Le) and 5′-trailer (Tr) sequences (at the genome ends). From the 3′-leader sequence, the genome encodes the nucleocapsid protein (NP), V protein (V), phosphoprotein (P), matrix protein (M), fusion protein (F), small hydrophobic protein (SH), haemagglutinin-neuraminidase (HN) and the large protein (L).

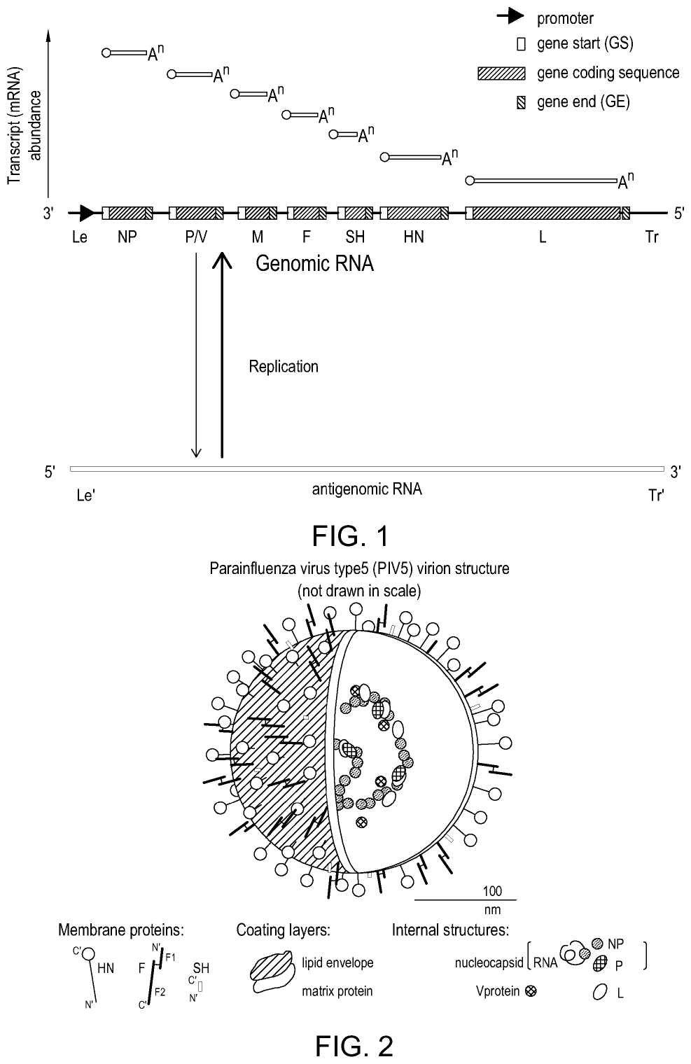

An exemplary PIV5 genome is shown in FIG. 1.

The HN, F and SH proteins protrude through the virus envelope; the HN protein binds the virus to its target cell, and together with the F protein, facilitates virus entry into the cell to initiate the replication process. The matrix protein is located on the inner surface of the virus envelope and plays an essential role in virion assembly. The genomic RNA is encapsidated by NP, forming a flexible helical nucleocapsid complex that is associated with the viral RNA-dependent RNA polymerase complex (vRdRP) consisting of L and P (see FIG. 2).

In view of the above, the disclosure provides a cohort of products derived from parainfluenza virus 5 (PIV5) and the genome thereof. All of the disclosed products may be for use as described above.

In one aspect, the viral genome derived product is a PIV5 vector. A PIV5 vector may comprise one or more wild-type PIV5 genes and a heterologous nucleic acid sequence for expression. Conversely, a PIV5 vector may (relative to a wild-type PIV5 genome) lack one or more wild-type PIV5 genes and comprise a heterologous nucleic acid sequence for expression.

The PIV5 vectors described herein may be used to generate recombinant proteins and/or to deliver therapeutic proteins.

The described PIV5 vectors are advantageous because:
They are capable of achieving high levels of heterologous protein expression in infected cells;
They can establish “persistent infections”-in other words, cells remain persistently infected;
Persistently infected cells contain many genomes, which if activated may lead to significantly enhanced expression;
Expression of multiple heterologous proteins in the same cell;
No problems with (cryptic) splicing of mRNA etc;
Can make vectors which do not produce infectious virus; and. Once vectors have been generated easy to use widely in vitro and in vivo.

By way of example, a PIV5 vector of this disclosure may lack, relative to a wild type PIV5 genome, one or more of the wild-type PIV5 gene(s). In one teaching a PIV5 vector of this disclosure may be characterised by, relative to a wild-type PIV5 genome, the full or partial deletion of any one or more of the wild-type genes.

In one teaching, a PIV5 vector of this disclosure may be characterised by the (functional) deletion of:
the F gene; and/or
the M gene; and/or
the P gene; and/or
the L gene; and/or
the HN gene.

The disclosed PIV5 vectors may be classed as single cycle vectors. Thus, a PIV5 genome derived product of this disclosure may be a single cycle vector (i.e. a PIV5 single cycle vector).

In one teaching, a PIV5 single cycle vector lacks, as compared to the wild-type PIV5genome, a functional copy of one or more of the wild-type PIV5 genes.

Without wishing to be bound by theory, although a single cycle vector can infect cells it cannot make infectious virus particles unless the missing protein(s) (encoded by the (functionally) deleted genes) are provided in trans during its replication. To facilitate this, helper cell-lines capable of (inducibly) expressing the missing protein(s) can be used. Expression of the missing protein may be inducible; for example expression may be induced by contact with, or addition of, an inducing agent.

In one teaching, a PIV5 vector of this disclosure may, relative to a wild-type PIV5 genome, comprise a (functionally) deleted F gene. A PIV5 vector which lacks the F gene may be referred to as single cycle vector and may be designated PIV5ΔF. While a PIV5ΔF vector can infect cells, it cannot make infectious virus particles unless the missing protein(s) (encoded by the (functionally) deleted genes) are provided during its replication. To facilitate this, helper cell-lines capable of (inducibly) expressing the missing protein(s) can be used. The helper cells in effect provide the missing protein(s) (encoded by the (functionally) deleted genes) in trans during replication. Without wishing to be bound by theory, it is suggested that PIV5ΔF vectors may be particularly useful where a high viral titre is preferred—for example for use in raising immune responses, inducing immune responses and/or for application in vaccine relate methods.

In one teaching, a PIV5 vector of this disclosure may, relative to a wild-type PIV5 genome comprise a (functionally) deleted M and F genes (thereby rendering it unable to express the M and F). A PIV5 vector which lacks the M and F genes may be referred to as single cycle vector and may be designated PIV5ΔM/F. As stated, a PIV5ΔM/F vector may be rescued by a helper cell line that expresses the missing (or deleted/functionally deleted) wild-type PIV5 genes in trans.

In one teaching, a PIV5 vector of this disclosure may, relative to a wild-type PIV5 genome comprise a (functionally) deleted M gene, F gene and HN gene (thereby rendering it unable to express the M, F and HN proteins). A PIV5 vector which lacks the M gene, F gene and HN gene may be referred to as single cycle vector and may be designated PIV5ΔM/F/HN. As stated, a PIV5ΔM/F/HN vector may be rescued by a helper cell line that expresses the missing (or deleted/functionally deleted) wild-type PIV5 genes in trans. In another teaching, a PIV5 vector of this disclosure may, relative to a wild-type PIV5 genome comprise (functionally) deleted M gene, F gene, P gene, L gene and HN gene (thereby rendering it unable to express the M, F, P, L and HN proteins). A PIV5 vector which lacks the M, F, P, L and HN gene may be referred to as single cycle vector and may be designated PIV5ΔM/F/P/L/HN. As stated, a PIV5ΔM/F/P/L/HN vector may be rescued by a helper cell line that expresses the missing (or deleted/functionally deleted) wild-type PIV5 genes in trans.

In a further teaching, a PIV5 vector of this disclosure may comprise, consist of or consist essentially of, a functional NP gene. Where the PIV5 vector comprises, consist of or consists essentially of a functional copy of the NP gene, any of the other (missing, deleted and/or functionally deleted) wild type PIV5 genes may (as described below) be provided in trans—for example by a helper cell which expresses those missing wild-type PIV5 genes (see description below). Without wishing to be bound by theory, it is suggested that by retaining the NP gene within the vector, the NP concentration builds up inside the helper cell and this facilitates the switch from “transcription mode” to a “replication mode”.

Any of the PIV5 vectors/single cycle vectors described herein, including for example PIV5ΔF and any PIV5 vector which lacks the M, F and HN genes, may further comprise a heterologous sequence for expression. The heterologous sequence may encode a therapeutic protein or a recombinant protein.

As such, the disclosure provides a PIV5 vector or PIV5 single cycle vector as described herein, comprising a heterologous sequence for expression in a cell, wherein relative to the wild-type PIV5 genome, the vector lacks one or more (functional) copies of a wild-type gene.

Without wishing to be bound by theory, it is suggested that the PIV5 vectors disclosed herein can establish persistent infections.

In order to make infectious particles, the vectors described herein, including the PIV5ΔF vectors and any PIV5ΔM/F/HN or PIV5ΔM/F/P/L/HN vectors, the missing PIV5 virus proteins may be provided in trans, for example through the use of helper cell lines. In the case of PIV5ΔF, only the F protein need be provided in trans. For PIV5ΔM/F/HN vectors the M, F and HN proteins need to be provided in trans. The present disclosure further provides helper cell lines which express the M and/or F and/or HN proteins (the exact helper cell PIV protein expression profile depending on the features of the corresponding PIV5 vector). These helper cell lines have been successfully used to rescue infectious particles that can in turn be used to infect cells in which the vector persists. Prior art methods of rescuing viral derived vectors may include methods which exploit the transient transfection of plasmids into cells. However, such methods result in a relatively low yield of “infectious” particles. In contrast, the yield infectious particles from a helper cell expressing the F protein is high (a high titre) similar to the wild type virus.

Any of the disclosed PIV5 vector(s)/PIV5 single cycle vector(s) may be used to deliver therapeutic proteins in vivo. Accordingly, disclosed is a method of administering a therapeutic protein, said method comprising administering a PIV5 vector/PIV5 single cycle vector according to this disclosure, which vector comprises a nucleic acid encoding the therapeutic protein for delivery. The method may be for in vivo and in vitro use. Where the method is for in vitro use, the administering step, may be replaced with a step in which a cell is contacted with a PIV5 vector or PIV5 single cycle vector of this disclosure.

The method may further require inducing the expression of the heterologous sequence.

In one teaching, PIV5 vectors of this disclosure and high titre PIV5 vectors, for example PIV5ΔF, can be grown in helper cell-lines. As stated, a helper cell line may (heterologously) express (via induction) the proteins which the PIV5 vector is unable to express. By way of example, where the vector is a PIV5ΔF vector, a helper cell may express the PIV5 F protein. This generates an infectious PIV5ΔF which can then be used to infect other cells (both in vitro and in vivo) to produce the required recombinant protein (for example an antibody). However, due to the lack of F protein expression, no infectious virus will be produced in these cells.

By way of a further example, where the vector lacks the M, F and HN genes, a helper cell may express the PIV5 M, F and HN proteins. This will generate an infectious PIV5 vector which can then be used to infect other cells (both in vitro and in vivo). However, due to the lack of M, F and HN protein expression, no infectious virus will be produced in these cells. In one teaching a vector of this disclosure (for example an Indel expression vector as described herein) expressing the relevant PIV5 viral protein (for example the F protein of PIV5) can be used as an alternative to a helper cell line.

A single cycle vector according to this disclosure, including, for example a PIV5ΔF vector is safe to use in any cell or system which does not (heterologously) express the necessary PIV proteins, for example (in the case of the PIV5ΔF vector), the PIV5 F protein. Indeed a single cycle vector according to this disclosure, including, for example a PIV5ΔF vector, is safe for use in patients with underlying health conditions and/or with cancer.

The single cycle vectors of this disclosure can be designed with an acute or persistent phenotype.

In another aspect, the invention provides a method of making a single cycle vector, said method comprising deleting or functionally deleting the F protein from the PIV genome.

This disclosure may provide helper cell lines. These helper cell lines may be engineered to express (potentially by induction) one or more of the PIV5 genes missing from the PIV vector. Thus the helper cell provides the PIV5 vector with the missing genes in trans.

A helper cell line may comprise any cell which is permissive to a PIV5 virus. For example, the helper cell may be a Vero cell.

A helper cell may express one or more PIV5 proteins. The PIV5 protein expression of the helper cell may correspond to the proteins that the PIV5 vector does not express. For example, a helper cell of this disclosure may be modified to express:
the PIV5 F protein; and/or
the PIV5 M protein; and/or
the PIV5 HN protein.

The disclosure provides a method of replicating a PIV5 vector of this disclosure, said method comprising culturing or replicating a PIV5 vector in a helper cell of this disclosure.

The disclosure provides a method of replicating a PIV5ΔF vector of this disclosure, said method comprising culturing or replicating a PIV5ΔF vector in a helper cell which expresses the PIV5 F protein.

The disclosure provides a method of replicating a PIV5ΔM/F/HN vector of this disclosure, said method comprising culturing or replicating a PIV5ΔM/F/HN vector in a helper cell which expresses the PIV5 M, F and HN protein.

In a further aspect the viral genome derived product is disabled helper paramyxovirus.

The viral genome derived product is disabled a defective interfering (DI) particle. DIs are subgenomic and often contain extensive deletions that render the DIs unable to complete a full replication cycle in the absence of a coinfecting, non-defective “helper” virus.

In one teaching, the viral genome derived product is a defective interfering (DI) particle derived from:
a Paramyxovirus;
PIV5; or
PIV5ΔF.

DIs derived from any of the above sources may be referred to as “copyback” DIs or “internal deletion” (Indel) DIs.

As such, this disclosure provides PIV5 derived copyback DIs.

The disclosure further provides PIV5 derived internal deletion (Indel) DIs.

In a copyback DI, the 3′ genomic promoter has been replaced by a sequence complementary to the 5′ antigenomic promoter; this is due to template switching from the antigenome to the nascent strand during synthesis of genomic RNA; as a consequence, the copyback DI cannot be transcribed. The termini of copyback DIs are thus complementary and form a dsRNA stem-loop structure when SDS treatment is used to dissociate the RNA genomes from encapsidating NP protein.

A PIV5 derived copyback DI of this disclosure may comprise the 3′ replication promoter of the PIV5 genome, duplicated and in the opposite orientation together with some of the L gene sequence.

A DI according to this disclosure may be obtainable by:
(i) passaging a wild type PIV5 in a cell; or
(ii) passaging:
   a single cycle vector of this disclosure; and/or
   a PIV5ΔM; and/or
   a PIV5ΔF/M; and/or
   a PIV5ΔF;
   in a relevant helper F cell.

The term ‘relevant helper F cell’ may comprise any cell which expresses the necessary missing PIV5 wild type genes. For a PIV5ΔM vector a helper cell may express the PIV5 M gene in trans (to provide the PIV5 M protein); for a PIV5ΔF/M vector, a helper cell may express the PIV5 F and M genes in trans (to provide the PIV5 F and M proteins) and for a PIV5ΔF vector, a helper cell may express the PIV5 F gene (to provide the PIV5 F protein) in trans.

In one teaching, the passaging may be done at a high multiplicity of infection.

Additionally or alternatively (and again without being bound by theory), it has been noted that a copyback DI may be best or most efficiently produced using a PIV5 virus/vector (including any vector of this disclosure-for example a single cycle vector; a PIV5ΔM, a PIV5ΔF/M or a PIV5ΔF vector) having an acute but not persistent phenotype. It is noted that a switch between a virus/vector with a persistent to acute phenotype may be achieved through the use of a single nucleotide change (resulting in single or point amino acid changes). For example, the inventors note a serine to phenylalanine change at position 157 in the PIV5 P protein (as encoded by the PIV5 P gene) can change a PIV5 virus/vector from having a persistent phenotype to having an acute/lytic phenotype (a limited number of other changes can achieve the same switch). This is in contrast to methods or uses for the preparation of cell lines which constitutively produce recombinant proteins, those cell lines may comprise vectors (of this disclosure) with a persistent phenotype.

Accordingly, a DI according to this disclosure may be obtainable by:
(i) passaging a wild type PIV5 with an acute phenotype in a cell; or
(ii) passaging:
   a single cycle vector with an acute phenotype of this disclosure; and/or
   a PIV5ΔM with an acute phenotype; and/or
   a PIV5ΔF/M with an acute phenotype; and/or
   a PIV5ΔF with an acute phenotype;
in a relevant helper F-expressing cells.

DIs obtainable by the above-described passaging method, may comprise copyback DIs.

A PIV5 derived copyback DI obtainable by the disclosed method may comprise the 3′ replication promoter of the PIV5 genome, duplicated and in the opposite orientation together with some of the L gene sequence.

A copy back DI of this disclosure may act as a potent inducer of: innate immune responses, aspects and features of the innate immune system and, for example, IFN.

A copyback DI of this disclosure may be:
(i) for use in medicine;
(ii) for use as medicaments;
(iii) for use in the treatment and/or prevention of acute or chronic diseases and infections; and
(iv) for use in the treatment and prevention of cancer.
(v) for use as an adjuvant;
(vi) for use as a vaccine adjuvant;
(v) for use in modulating an immune response and/or an innate immune response.

The disclosure further provides a method of modulating, improving or augmenting the immune response to an antigen or vaccine, said method comprising immunising a subject with the vaccine or antigen and a DI of this disclosure.

Moreover, the disclosure provides a method of treating or preventing:
acute or chronic diseases and infections; or
cancer;
said method comprising administering a subject in need thereof, a therapeutically effective amount of a DI/copyback DI of this disclosure.

The term ‘subject’ may refer to any human or animal subject having or suspected of having any relevant disease, condition, infection or cancer. The term ‘subject’ may further embrace any human or animal subject predisposed or susceptible to any relevant disease, condition, infection or cancer.

It should be noted that Copyback DIs do not express any viral proteins (and cannot currently be used as expression vectors); they are generated by mistakes during virus replication. Therefore, and without wishing to be bound by theory, it is suggested that PIV5 copy-back DIs are safe to use in vivo, including in immunosuppressed individuals.

A PIV5 copy-back DI of this disclosure may be for use in the induction of an innate immune response both in vitro and in vivo. Without wishing to be bound by theory, the engagement of a PIV5 derived copy-back DI with pathogen recognition receptors (PRRs) activates a number of cellular kinases and transcription factors (e.g., IRF3, NF-κB) that regulate the expression of several cytokines, including, for example, IFNs, tumour necrosis factor (TNF), and interleukin 6 (IL-6), and can stimulate DC maturation and enhance antigen specific immunity to pathogen associated antigens.

In another aspect the viral genome derived product is an Indel vector (this may also be referred to as an Indel DI).

Indel vectors do not express any viral (PIV5) proteins but retain the 3′ and 5′ ends of the virus genome (with the Le and Tr sequences) and therefore possess at least the transcription and replication signals essential for virus transcription and replication.

Transcription and replication of the disclosed Indel vectors is dependent on additional viral proteins being expressed either in trans, for example in helper cell-lines, or by co-infection with helper viruses including, for example helper virus products derived from PIV5.

A potential advantage of such vectors is that they have a very high coding capacity. For example, the coding capacity of any of the PIV5 vectors described herein may include heterologous sequences in the region of 16-20 kb. One of skill will appreciate that the size of a heterologous insert may depend on the number and size of the PIV5 genes retained.

The disclosed ‘Indel’ vectors may be to express heterologous sequences (e.g. genes, or combination of genes) at sizes up to, for example 16-20 kb.

Thus in one teaching, the viral genome derived product is an Indel vector derived from a Paramyxovirus, for example, PIV5.

PIV5 derived Indel vector of this disclosure may comprise a nucleic acid sequence which, relative to a wild-type PIV5 genome, lacks all of the NP, P/V, M, F, SH, HN and/or L genes. As stated, a PIV5 Indel vector may retain (or comprise) the 3′ Le and 5′ Tr sequences of PIV5 viral genome (i.e. the ends comprising the Le and Tr sequences).

A PIV5 Indel vector may further comprise one or more heterologous nucleic acid sequences. The heterologous sequence(s) may encode recombinant and/or therapeutic proteins. The heterologous sequence(s) may be for expression in a cell. The heterologous sequence(s) may be inducibly expressible. A PIV5 derived Indel vector of this disclosure may comprise, or further comprise a nucleic acid sequence encoding a heterologous protein. It should be understood that the Indel vectors of this disclosure can be modified to receive almost any heterologous sequence encoding almost any sort of heterologous protein. For example, the Indel vectors of this disclosure may comprise, for example, heterologous nucleic acid sequences which encode any one or more of the following categories of protein:
antigens (including viral and/or bacterial antigens);
tumour specific antigens;
multivalent CTL antigens;
recombinant proteins (for expression);
components of the immune system;
immunomodulatory compounds;
antibodies (including fragments and/or parts thereof);
cytokines.

By way of example only, a PIV5 derived Indel vector of this disclosure may comprise a nucleic acid sequence encoding a SRAS CoV-2 antigen for example the SRAS-CoV-2 spike protein.

In a further example, the PIV5 derived Indel vector of this disclosure may comprise a nucleic acid sequence encoding interferon (IFN).

In a further teaching, a PIV5 derived Indel vector of this disclosure may comprise a nucleic acid sequence encoding all or part of an antibody. The PIV5 derived Indel vector may be modified to express the heavy and/or light chain of an antibody or any fragment(s) thereof. In one teaching, the PIV5 derived Indel vector may be modified to include nucleic acid encoding the heavy and/or the light chain(s) of the humanised anti-V5 mAb.

A PIV5 derived Indel vector of this disclosure may comprise or further comprise an internal ribosome entry site (IRES).

The PIV5 derived Indel vector of this disclosure may comprise or further comprise a nucleic acid sequence encoding a heterologous reporter moiety, for example an optically detectable reporter moiety such as a fluorescent protein. Examples may include nucleic acid sequences which encode fluorescence proteins such as mCherry and/or GFP.

A PIV5 derived Indel vector of this disclosure may comprise, or further comprise a nucleic acid sequence for use in a method of selection or enrichment. For example, A PIV5 derived Indel vector of this disclosure may comprise, or further comprise an antibiotic resistance gene. Examples may include genes which encode an enzyme which inactivates or neutralises an antibiotic. A PIV5 derived Indel vector of this disclosure may comprise, or further comprise a nucleic acid sequence encoding enzymes which provide blasticidine and/or puromycin resistance.

A PIV5 derived Indel vector of this disclosure may comprise or further comprise a nucleic acid encoding a reporter gene, for example an optically detectable moiety, a fluorescent protein. Examples may include nucleic acid sequences which encode fluorescence proteins such as mCherry and/or GFP.

A PIV5 derived Indel vector of this disclosure may comprise or further comprise an internal ribosome entry site (IRES).

An Indel DI of this disclosure may find application as an expression vector for in vitro and in vivo use. An Indel DI of this disclosure may be used in the production of recombinant/therapeutic proteins.

Without wishing to be bound by theory, Indel DIs do not encode any viral proteins and are thus unable to replicate or be packaged into infectious particles without the co-expression of the appropriate viral replication and/or structural proteins. These co-expressed and essential proteins can be provided by:
coinfecting with (non-defective) “helper” virus;
coinfection with mini-replicon vectors that express the replication proteins;
transient transfection with expression plasmids that express the required viral proteins; and/or
the provision of making helper cell-lines which express the appropriate viral proteins.

Helper cell-lines which inducibly co-express, for example, any of the M, F, NP, V, P, L and/or HN PIV5 proteins may facilitate the replication of any of the disclosed Indel DIs—in turn this will facilitate the expression of heterologous proteins in the absence of co-infecting virus.

The disclosure further provides helper cell-lines which inducibly co-express all the PIV5 viral proteins. Cell lines of this type may facilitate the replication and packaging of DIs in the absence of co-infecting virus.

The present disclosure provides the disclosed copyback DIs for use in (directly or indirectly) modulating gene expression. The expression of one or more of the following genes 49 genes (see table 1) may be modulated by a copyback DI of this disclosure. The modulation may be direct modulation or indirect modulation occurring via the action of multiple pathways and/or cytokines in response to the presence of a copyback vector of this disclosure.

The modulated gene expression may occur in a cell, for example a cancer cell. The modulated gene expression may occur in an adenocarcinomec human alveolar basal epithelial cell, for example an A549 cell.

TABLE 1

| No | Gene |
|---|---|
| 1 | KCNJ18 |
| 2 | FAM45A |
| 3 | RSAD2 |
| 4 | CYP2D6 |
| 5 | IFNL2 |
| 6 | TNFSF12-TN |
| 7 | IFNL1 |
| 8 | C10orf32-AS |
| 9 | IFIT2 |
| 10 | PLEKHA4 |
| 11 | CH25H |
| 12 | IFNL4 |
| 13 | DHX58 |
| 14 | OASL |
| 15 | IFIT1 |
| 16 | IFNB1 |
| 17 | HCAR2 |
| 18 | OAS2 |
| 19 | FSBP |
| 20 | LRRN3 |
| 21 | SAMD9L |
| 22 | IFIH1 |
| 23 | XAF1 |
| 24 | IFIT3 |
| 25 | MX1 |
| 26 | HCAR |
| 27 | GBP1 |
| 28 | LRP2 |
| 29 | TAC3 |
| 30 | RAET1L |
| 31 | MX2 |
| 32 | TNFSF10 |
| 33 | IL6 |
| 34 | CMPK2 |
| 35 | GBP4 |
| 36 | IDO1 |
| 37 | CCL5 |
| 38 | SAMD9 |
| 39 | DDX58 |
| 40 | ZC3HAV1 |
| 41 | IFI27 |
| 42 | IFI44/IFI44L |
| 43 | TNFSF13B |
| 44 | ISG15 |
| 45 | HERC5 |
| 46 | FAM65B |
| 47 | IFI27 |
| 48 | IFNL3 |
| 49 | DDX60 |

The present disclosure further provides a method of modulating the expression of any one or more of the above disclosed genes, said method comprising contacting the gene the expression of which is to be modulated, with a disclosed copyback DI.

The present disclosure further provides a (in vitro or in vivo) method of modulating the expression of any one or more of the above disclosed genes in a cell (for example a cancer cell, an adenocarcinoma human alveolar basal epithelial cell or an A549 cell), said method comprising contacting the cell with a disclosed copyback DI.

The term modulation may embrace any up or down regulation of the expression of any of the genes listed above. For example, the disclosed copyback DIs may be used to upregulate the expression of any one or more of the above listed genes.

Without wishing to be bound by theory, the disclosed copyback DIs may initiate innate signalling through the RIG-I/mda5 pathway; this may lead to the transcriptional activation of many cellular immune genes in addition to IFN genes.

### DETAILED DESCRIPTION

The present invention will now be disclosed by reference to the following Figures which show:

FIG. 1: The Parainfluenza virus type 5 genome: PIV5 has a non-segmented, negative-sense RNA genome of 15,246 nucleotides (nt) containing seven tandemly arranged genes, that encode eight proteins, flanked by 3′-leader and 5′-trailer sequences at the genome ends. From the 3′-leader sequence, the genome encodes the nucleocapsid protein (NP), V protein (V), phosphoprotein (P), matrix protein (M), fusion protein (F), small hydrophobic protein (SH), haemagglutinin-neuraminidase (HN), and the large protein (L).

FIG. 2: Schematic diagram of the PIV5 virus particle (virion). The PIV5 genomic RNA is encapsidated by NP, forming a flexible helical nucleocapsid complex that is associated with the viral RNA-dependent RNA polymerase complex (vRdRP) consisting of L and P. Also associated with the nucleocapsid is the viral interferon antagonist, the V protein. The nucleocapsid is surrounded by viral envelope, through which protrude the HN, F and SH proteins; located on its inner surface is the matrix protein.

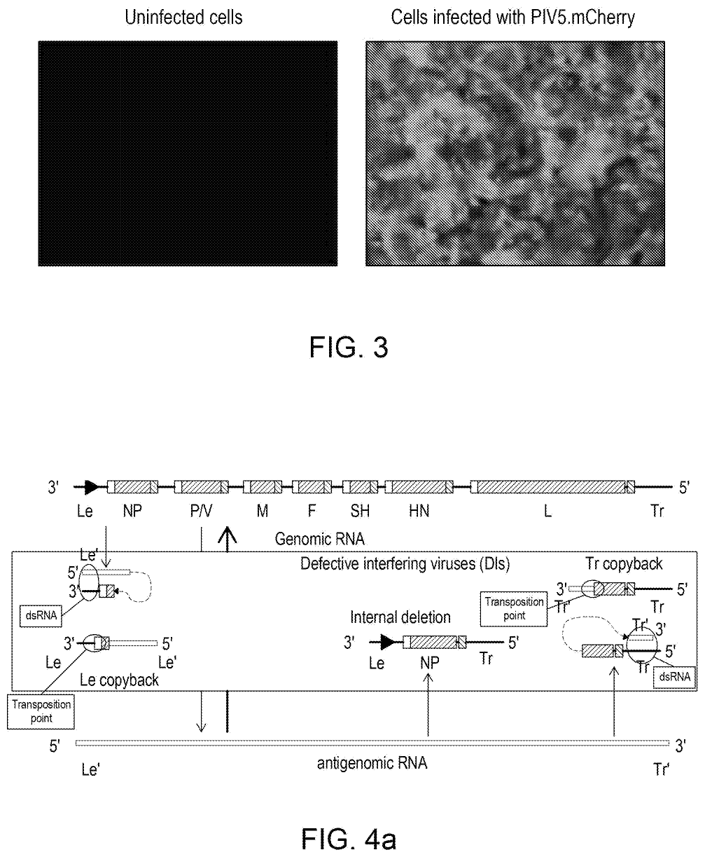

FIG. 3. Vero cells were or were not infected with PIV5.mCherry at a high moi and images taken 24 h p.i.

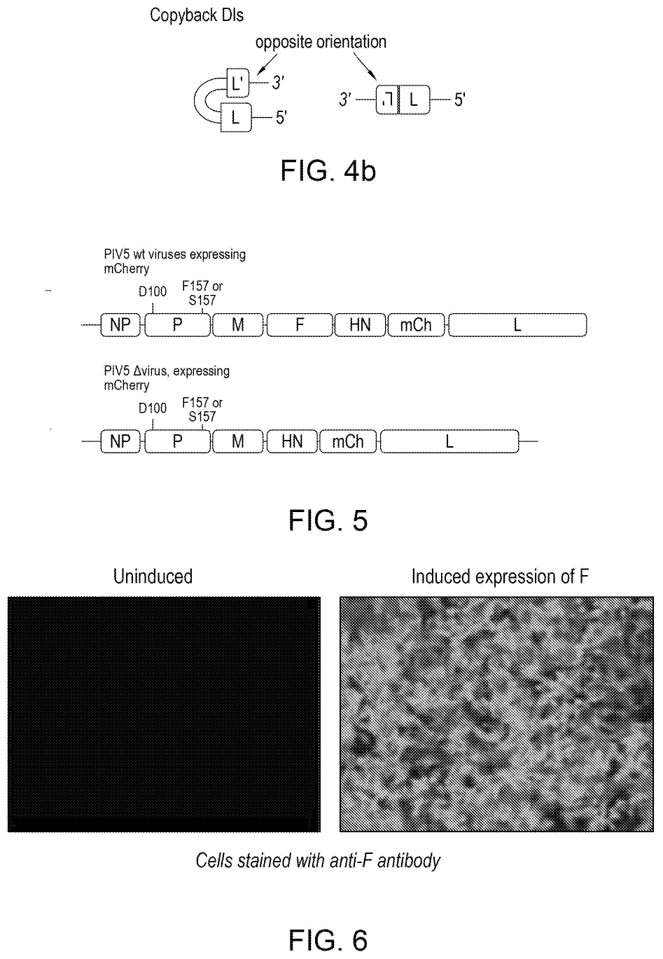

FIG. 4: The genomic structure of internal deletion and copyback DIs. 4b: In copyback DIs the 3′ replication promoter of the virus genome is duplicated (in the opposite orientation) together with some of the L gene sequence.

FIG. 5: Genome structure of PIV5ΔF compared to wild type (wt) PIV5.

FIG. 6: Inducible expression of the PIV-5 F protein by addition of doxycycline (Dox) to the culture medium of BSRT7.F helper cell-line (note: in this figure, the cells are stained red, not because they express mCherry, but because the expressed F protein was visualized by immunofluorescence using a Texas Red conjugated secondary antibody).

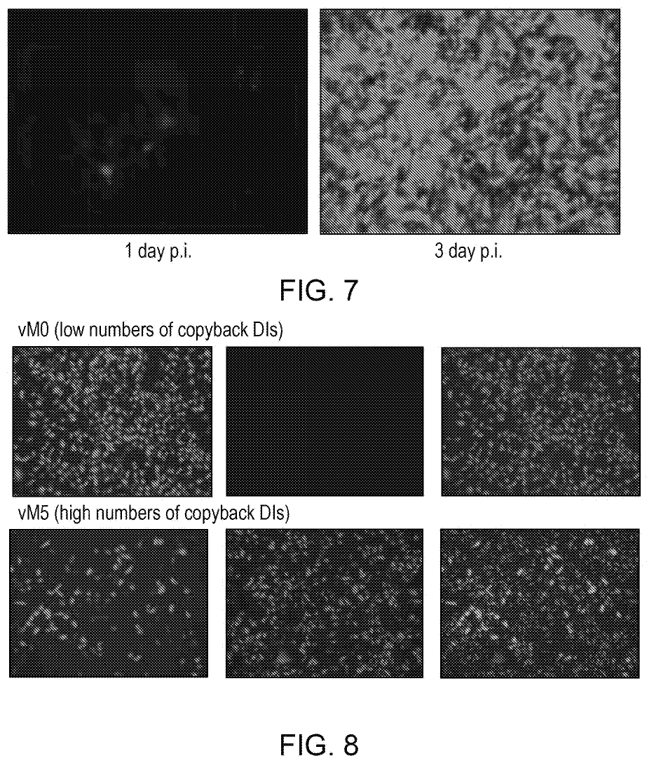

FIG. 7: Replication of rPIV5ΔF.mcherry in BSRT7.F helper cell lines. Helper cells expressing F were infected at a low multiplicity of infection and images taken at 1 and 3 days p.i. Note the increase in number of infected cells between 1 and 3 days post infection. The virus does not spread in cells unless they express F.

FIG. 8: A549pr(IFN-β).GFP reporter cells were infected with preparations of PIV5.mCherry containing low (vM0) or high (vM5) numbers of copyback DIs. Images of mCherry and GFP expression were taken at 24 h p.i. (Note expression of GFP is under the control of the IFN-β promoter.)

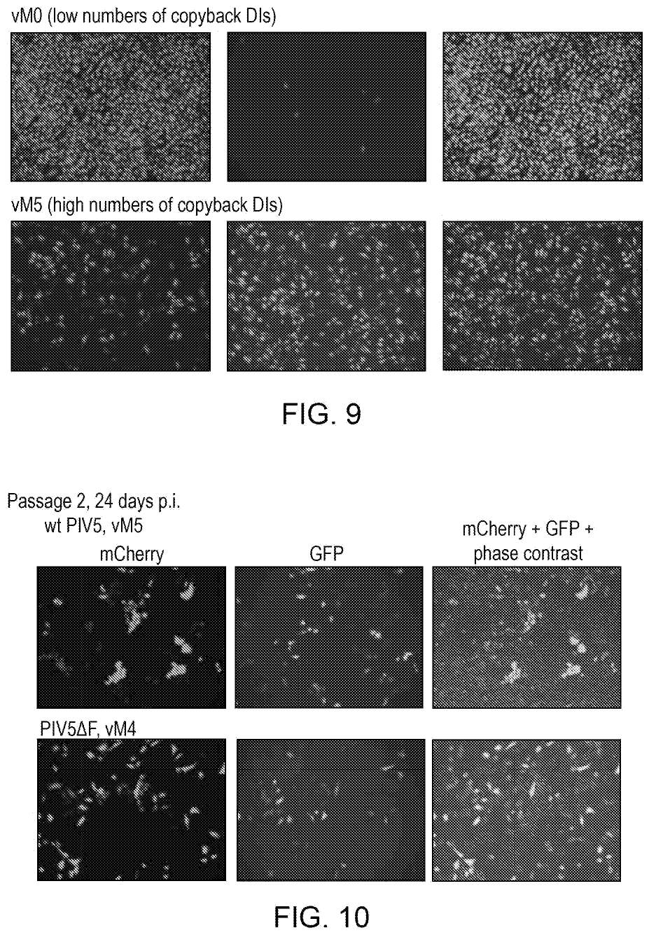

FIG. 9: As described for FIG. 8 except that the A549pr(IFN-β).GFP reporter cells were infected with preparations of PIV5ΔF.mCherry containing low (vM0) or high (vM5) numbers of copyback DIs.

FIG. 10: A549 IFN-β reporter cells infected with PIV5 containing high numbers of copyback DIs (see FIGS. 7 and 8) continue to activate innate immune signalling pathways more than 24 days post infection.

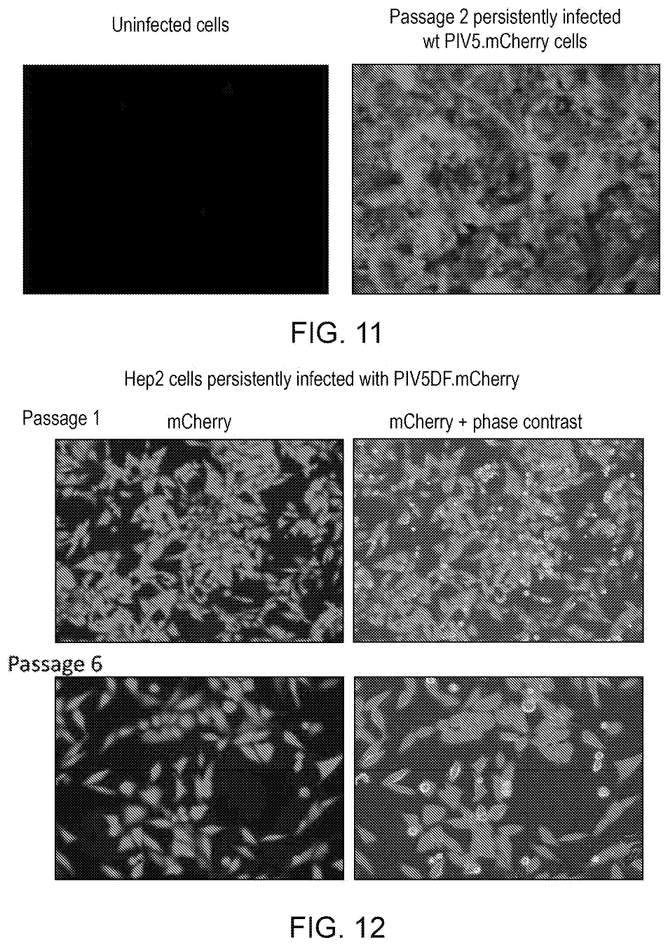

FIG. 11: Vero cells persistently infected with wt PIV5.mCherry continue to express mCherry after 6 passages.

FIG. 12: >95% of Hep2 cells persistently infected with PIV5ΔF.mCherry continue to express mCherry at passage 6.

FIG. 13: Cells infected with PIV5ΔF.mCherry make higher levels of mCherry than cells in which expression of mCherry is under a Dox inducible promoter. mCherry expression in a an mCherry inducible cell-line following its induction with Dox and in the same cell-line (in the absence of Dox induction) following with PIV5ΔF.mCherry.

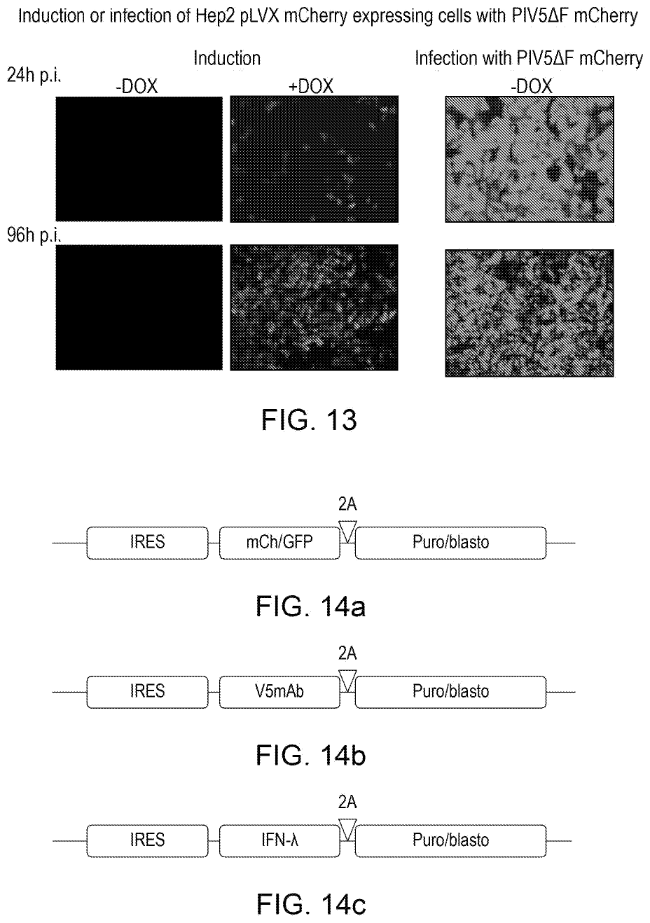

FIG. 14: Example of genome structures of internal deletion vectors for the expression of heterologous/therapeutic proteins, including mCherry, GFP, heavy and light chains of the humanised anti-V5 mAb and interferon-A. Additional possible genes for cloning include tumour specific antigens, multivalent CTL antigens, recombinant proteins etc.

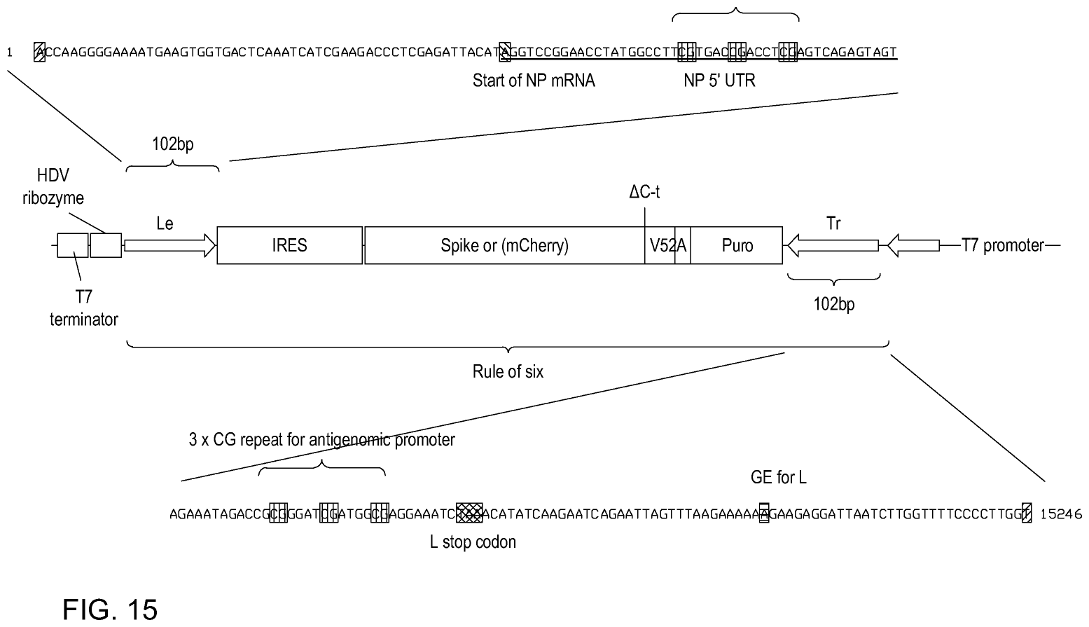

FIG. 15: Detailed genome structures of an internal deletion of vector we constructed for the expression of mCherry (and the spike protein of SARS.Cov2).

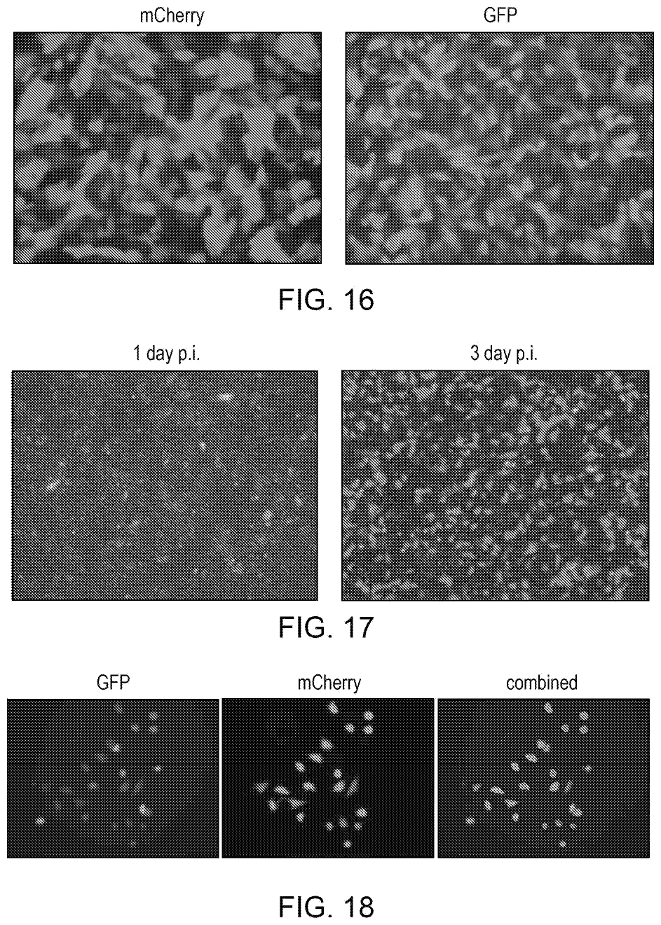

FIG. 16: Indel vectors can be rescued by co-infection with wild type (wt) PIV5 and express heterologous proteins as exemplified by expression of mCherry and GFP. Vero cells infected with wt PIV5 and Indel vectors expressing either mCherry or GFP.

FIG. 17: Indel vector expressing the F protein can support the replication of PIV5ΔF.mCherry viruses. Vero cells were co-infected with a low moi of Indel F and PIV5ΔF.mCherry. Note the increase in the number of mCherry positive cells between 1 and 3 days post infection. In the absence of heterologous expression of F by the Indel vector PIV5ΔF.mCherry does to spread from cell to cell.

FIG. 18: Cells can be co-infected with different Indels, thereby potential increasing the number of recombinant proteins expressed in individual cells. Vero cells were co-infected with Indel GFP (blasticidine) and Indel mCherry (puromycin) that had been rescued with wt PIV5. Co-expressing cells were isolated by culturing the co-infected cells in the presence of puromycin and blasticidine.

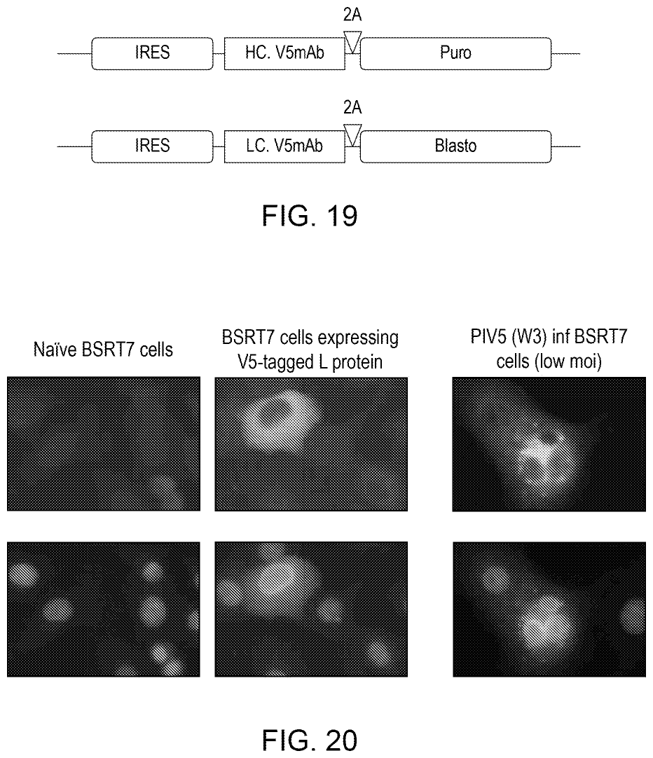

FIG. 19: Indel vectors constructed for the independent expression of the heavy and light chains of the humanised anti-V5 antibody.

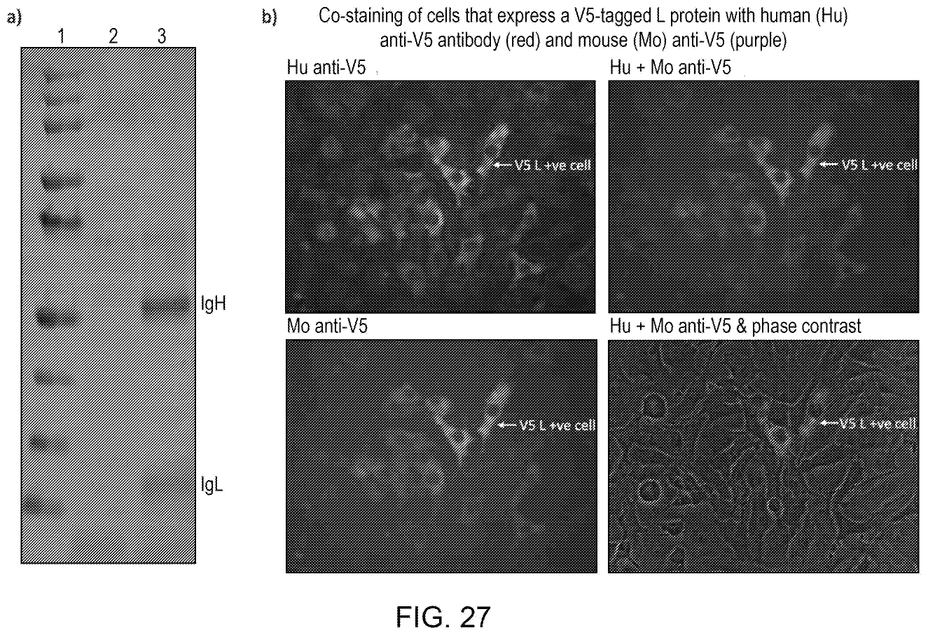

FIG. 20: The humanised anti-V5 antibody produced by the Indel vectors is functional as demonstrated by its ability to immunestaining cells expressing a V5 tagged version of the L (see also FIG. 27). Cells were indirectly stained with the human anti-V5 antibody (red) made by Indel vectors and countered stained with DAPI (blue).

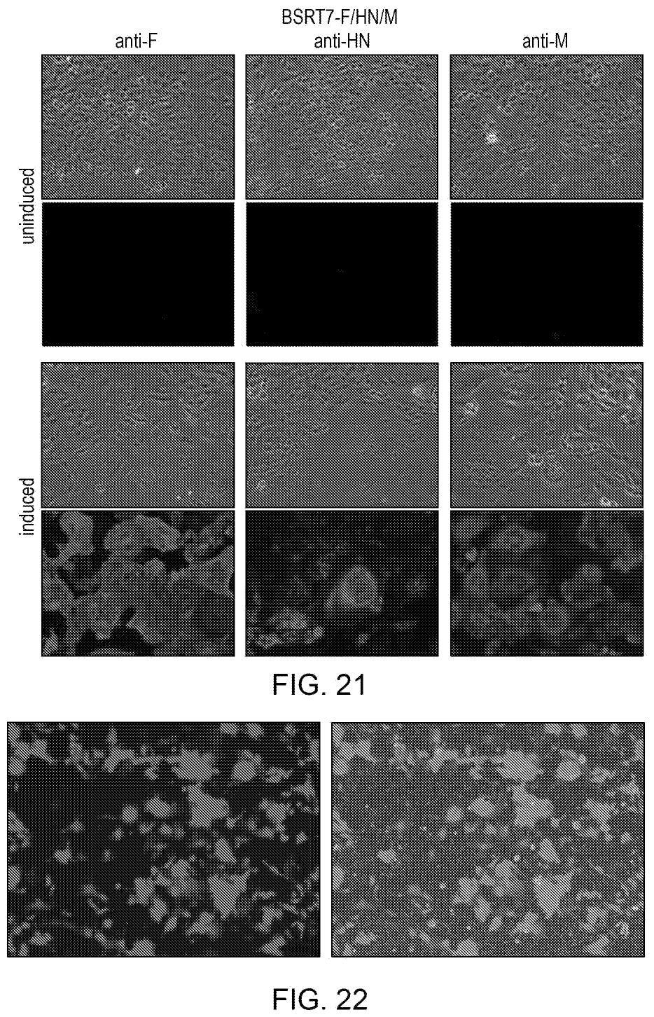

FIG. 21: A cell-lines in which M, F and HN are co-expressed following induction with Dox. Expression of M, F and HN were visualised by immunofluorescence using specific monoclonal antibodies.

FIG. 22: Mini-replicons (ΔM-HN) that express the replication proteins can be used as helper vectors for the rescue of Indel vectors, and can be co-packaged into infectious particles in the M, F and HN cell-line (FIG. 21) to infect other cells. Figure shows the rescue and packaging of mini replicon vector (ΔM-HN)+Indel mCherry (blasticidin) in BSRT7-M/F/HN cells+DOX (3 days post transfection).

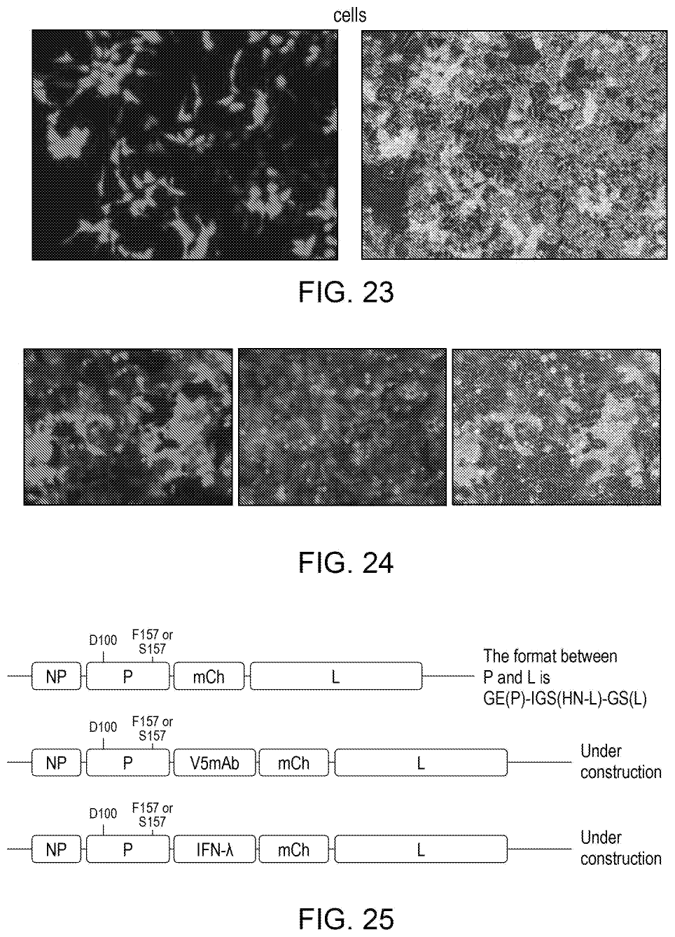

FIG. 23: BSRT7-M, F and HN expressing cells infected with ΔM-HN+Indel mCherry vectors produced from cells as described in FIG. 22.

FIG. 24: Hep2.GFP cells infected with ΔM-HN+Indel mCherry (blasticidin) produced from BSRT7-M, F and HN cells. [Note GFP +ve cells that are also positive for mCherry confirm that the mini replicon and Indel.mCherry vectors have been packaged into infectious particle in the BSRT7-M, F and HN cell-line.

FIG. 25: Schematic diagrams of mini-replicon vectors that have, or are being constructed, for the expression of heterologous proteins in vitro and in vivo. Minireplicons expressing mCherry, IFN-λ or anti-V5mAb. Additional possible genes for cloning include tumour specific antigens, multivalent CTL antigens, recombinant proteins etc.

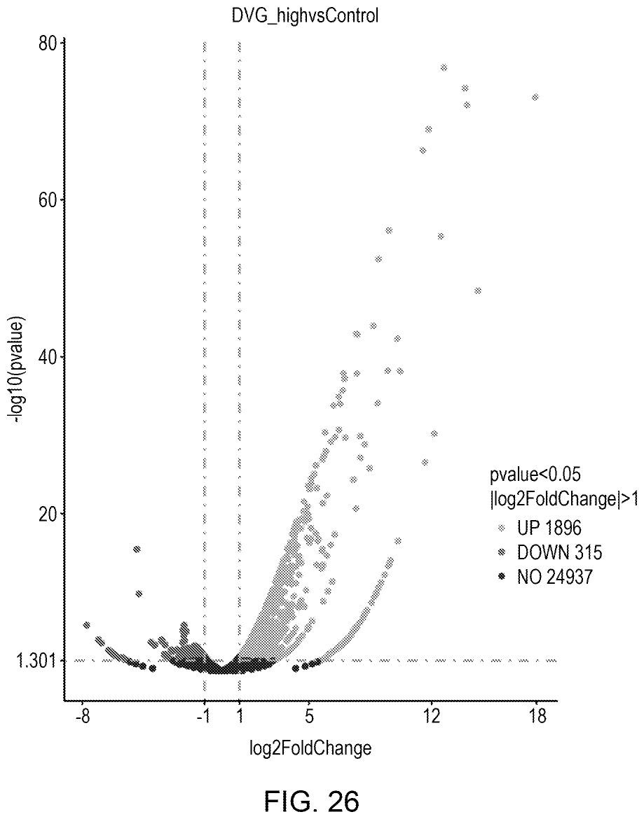

FIG. 26: A549 cells were, or were not, infected with a copyback DI-rich preparation of PIV5 in the presence of cycloheximide. At 6 h post infection the cells were harvested into Trizol and gene expression compared by high throughput sequencing (see also Table 1).

FIG. 27. Expression of the humanised anti-V5 antibody by a PIV5ΔF expression vector. CHO cells were infected at a high moi with PIV5ΔF.Hu anti-V5 and 3 days post infection the culture medium was collected. Panel a) Antibody present in the culture medium was captured on fixed and killed suspension Staphylococcus Cowan strain A and the heavy (IgH) and light (IgL) chains visualised by Coomassie Staining following SDS-PAGE (lane 3: lane 2 St A only, lane 1 molecular weight markers). Panel b), humanised anti-V5 antibody produced by PIV5ΔF. Hu anti-V5 vector is functional. BSRT7.L cells, in which approximately 30% of the cells express a V5-tagged version of the L protein following induction with Dox, was co-stained with human anti-V5 (red) and mouse anti-V5 (purple). Note the co-staining confirms that the human anti-V5 antibody is functional.

This disclosure provides:
PIV5ΔF copyback DIs (and similar approaches with other disabled helper paramyxoviruses) for therapeutic purposes, including vaccine adjuvants, cancer therapy and the treatment of certain acute and chronic infectious diseases.
PIV5 vectors which are able to establish persistent infections for the production of recombinant proteins in vitro and as means of delivering therapeutic proteins in vivo.
provides PIV5 internal deletion mutants as vectors for in vitro and in vivo production of recombinant/therapeutic proteins.
Internal DIs which express helper proteins, for example the PIV F protein, as method to complement deletion mutants of PIV5, for example PIV5ΔF, and for delivery of therapeutic proteins/vaccines.
   Generation of PIV5 mCherry Viruses

To further study the molecular biology of PIV5 and its interaction with cells we have generated a number of recombinant viruses that express mCherry facilitating the rapid and easy identification of infected cells (FIG. 3).

### Defective Interfering Genomes of PIV5 and Other Paramyxoviruses

Paramyxoviruses, including PIV5, spontaneously generate defective interfering virus genomes (DIs) due to errors during replication. These DIs are subgenomic and contain deletions (often extensive) that render the DIs unable to complete a full replication cycle in the absence of a coinfecting, non-defective “helper” virus. Paramyxovirus DIs may be “internal deletion” or “copyback” and these two types of DIs differ considerably in their genome structures (FIG. 4a/b).

Internal deletion DIs retain the Le and Tr sequences of the genome and therefore possess transcription and replication signals and have been shown to generate viral translation products. In contrast, the 3′ genomic promoter in trailer copyback DI [DI(TrCB)] genomes has been replaced by a sequence complementary to the 5′ antigenomic promoter (and therefore cannot be transcribed) due to template switching from the antigenome to the nascent strand during synthesis of genomic RNA; the termini of DI(TrCB)s are thus complementary and form a dsRNA stem-loop structure when SDS treatment is used to dissociate the RNA genomes from encapsidating NP protein. This structure is thought to be responsible for the ability of DI(TrCB)s to act as potent inducers of IFN.

### Copyback DIs are Powerful Inducers of Innate Immune Responses Both In Vitro and In Vivo.

Paramyxoviruses are poor activators of early innate immunity for two main reasons. First, they encode interferon (IFN) antagonists that can both inhibit the activation of the IFN-induction cascade and can block IFN signalling. In the case of PIV5, its IFN antagonist, the V protein, interacts with, and blocks the activity of melanoma differentiation-associated protein 5 (MDA5), as well as binding to the protein called laboratory of genetics and physiology 2 (LGP2) to negatively regulate retinoic acid-inducible gene I (RIG-I). In addition, PIV5 V targets STAT1 for proteasome-mediated degradation to block IFN-signalling.

Paramyxoviruses also tightly control virus transcription and replication, thereby limiting the production of pathogen-associated molecular patterns (PAMPs) that active pathogen recognition receptors (PRRs) and the IFN response. However, during replication paramyxoviruses make mistakes, including the generation of copyback DVGs. Copyback DVGs are powerful inducers of innate immune responses both in vitro and in vivo. DVG engagement of PRRs activates a number of cellular kinases and transcription factors (e.g., IRF3, NF-κB) that regulate (directly or indirectly) the expression of several cytokines, including IFNs, tumour necrosis factor (TNF), and interleukin 6 (IL-6), and can stimulate DC maturation and enhance antigen specific immunity to pathogen associated antigens.

To visualise the ability of viruses to induce innate immune responses we generated a cell-line that expresses GFP following activation of a number of innate intracellular responses, including the IFN pathway. Using this cell-line we showed that the IFN-induction cascade was only activated in a very few cells infected with preparations PIV5 that had few copyback DIs. In contrast, preparations of PIV5 rich in copyback DIs activate the IFN-response in most infected cells (FIG. 5).

### Potential Therapeutic Uses of Paramyxovirus Copyback DIs.

Given the ability of paramyxovirus copyback DIs to induce powerful innate immune responses in vivo, it has been suggested that they may be used therapeutically in a variety of clinical settings, for example:
i. As vaccine adjuvant
ii. For inducing anti-tumour activity in cancer patients
iii. Induction of innate immune responses for the treatment of certain acute and persistent infections.

A disabled PIV5 vector of this disclosure may have a range of therapeutic purposes; this includes generation of copyback DIs for use in cancer patients. The fusion (F) protein of PIV5 is essential to initiate entry of the virus into cells for replication to occur. To develop a safe PIV5 vector that could be used in patients with underlying health issues, such as cancer patients, the F gene was deleted from PIV5, generating a vector termed PIV5ΔF (FIG. 5).

Whilst PIV5ΔF can infect cells it cannot make infectious virus particles unless the F protein is provided in trans during its replication. To facilitate this, helper cell-lines are provided. In these cell-lines, the expression of F is induced by the addition of Dox to the tissue culture medium (FIG. 6).

Infectious PIV5ΔF can be readily grown in these F-helper cell-lines (FIG. 7). Such infectious PIV5ΔF can then be used to infect other cells, both in vitro and in vivo. However, no infectious virus will be produced in these cells (due to the lack of expression of F).

Generation of Copyback DIs with wt PIV5 and PIV5ΔF

By passaging wt PIV5 in tissue culture cells, and PIV5ΔF in helper F-expressing tissue culture cells, at high multiplicities of infection, copyback DIs are generated that are powerful inducers of innate immune responses (FIGS. 8 and 9). Preparations of wt PIV5 and PIV5ΔF rich in copyback DIs may be used therapeutically in a number of clinical settings.

### PIV5 Vector Uses

The disclosed PIV5 vectors able to establish persistent infections for the production of recombinant proteins in vitro and may be used to delivering therapeutic proteins in vivo.

There are a number of advantages over other (prior art) systems

### In Vitro

High levels of expression of heterologous proteins in persistently infected cells.
Persistently infected cells contain many genomes, which if activated may lead to significantly enhanced expression.
Expression of multiple heterologous proteins in the same cell.
No problems with (cryptic) splicing of mRNA etc.
Can make vectors which do not produce infectious virus.
Once vectors have been generated easy to use widely in vitro and in vivo.

### In Vivo

Potential to establish prolonged/persistent infections in vivo for production of heterologous proteins/long-lasting immunity (vaccines).
Can infect most cell types, including immune cells.
Can be manipulated to be powerful activators of innate immune responses.
No DNA involved in the expression of heterologous proteins in vivo.
Can make non replicating vectors for use in immunosuppressed patients.
PIV5 has already received regulatory approval for use in humans.

PIV5, unlike most other RNA viruses, can readily and immediately establish persistent infections in tissue culture cells. Persistently infected tissue culture cells can be readily passaged. Cell-lines can be readily made that persistently express heterologous proteins following infection with a variety of recombinant PIV5 viruses, including wt PIV5 and PIV5ΔF, exemplified by expression of mCherry, respectively (see FIGS. 11 and 12).

### Development of PIV5 Internal Deletion Mutants as Vectors for In Vitro and In Vivo Production of Recombinant/Therapeutic Proteins.

Internal deletion (Indel) DIs retain the Le and Tr sequences of the genome and therefore possess transcription and replication signals and can therefore be developed as novel expression vectors for in vitro and in vivo production of recombinant/therapeutic proteins. Indel DIs do not encode any viral proteins and are thus unable to replicate or be packaged into infectious particles without the co-expression of the appropriate viral replication and structural proteins. These proteins can be provided by:
i. Coinfecting with non-defective “helper” virus.
ii. Coinfection with mini-replicon vectors that express the replication proteins
iii. Transient transfection with expression plasmids that express the required viral proteins.
iv. By making helper cell-lines expressing the appropriate viral proteins, e.g. the F protein facilitating the rescue of PIV5ΔF or other cell-lines which inducibly co-express the M, F, HN, NP, V, P or L proteins or any combinations thereof; these will facilitate the replication of DIs and the expression of heterologous proteins in the absence of co-infecting virus. Cell-lines that inducibly co-express all the viral proteins, will facilitate the replication and packaging of DIs in the absence of co-infecting virus.
   Expression of PIV5 F Protein from an Indel PIV5 Vector.

An Indel expression vector that express the F protein of PIV5 can be used to rescue PIV5ΔF.mCherry and may be an alternative method to using helper cell-lines for the rescue PIV5ΔF vectors for the production of recombinant/therapeutic proteins in vitro and in vivo.

Indel Vectors Can be Replicated in Cells Co-Infected with PIV5 Min-Replicons That Express the NP, P and L Proteins.

Mini replicons can replicate independently of helper virus as they express the replication proteins (NP, P and L) but they cannot be packaged into infectious virus particles as they do not express the structural M, F and HN proteins. However, mini-replicons can be rescued and packaged into infectious particles by transient expression of the M, F and HN proteins. Alternatively, disclosed are cell-lines which inducibly express the M, F and HN proteins (FIG. 21).

Sequences

pBH276ΔF.mCherry
4 configurations:
W3A (shown)
D100
F157
D100/F157
CATGTGAGCAAAAGGCCAGCAAAAGGCCAGGAACCGTAAAAAGGCCGCGTTGCTGGCGTTTTTCCATA
GGCTCCGCCCCCCTGACGAGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCCTGTTCCGACCCTGCCGCT
TACCGGATACCTGTCCGCCTTTCTCCCTTCGGGAAGCGTGGCGCTTTCTCATAGCTCACGCTGTAGGT
ATCTCAGTTCGGTGTAGGTCGTTCGCTCCAAGCTGGGCTGTGTGCACGAACCCCCCGTTCAGCCCGAC
CGCTGCGCCTTATCCGGTAACTATCGTCTTGAGTCCAACCCGGTAAGACACGACTTATCGCCACTGGC
AGCAGCCACTGGTAACAGGATTAGCAGAGCGAGGTATGTAGGCGGTGCTACAGAGTTCTTGAAGTGGT
GGCCTAACTACGGCTACACTAGAAGAACAGTATTTGGTATCTGCGCTCTGCTGAAGCCAGTTACCTTC
GGAAAAAGAGTTGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTTCTACGGGGTCTG
ACGCTCAGTGGAACGAAAACTCACGTTAAGGGATTTTGGTCATGAGATTATCAAAAAGGATCTTCACC
TAGATCCTTTTAAATTAAAAATGAAGTTTTAAATCAATCTAAAGTATATATGAGTAAACTTGGTCTGA
CAGTTACCAATGCTTAATCAGTGAGGCACCTATCTCAGCGATCTGTCTATTTCGTTCATCCATAGTTG
CCTGACTCCCCGTCGTGTAGATAACTACGATACGGGAGGGCTTACCATCTGGCCCCAGTGCTGCAATG
ATACCGCGAGACCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGGGAAGCTAGAG
TAAGTAGTTCGCCAGTTAATAGTTTGCGCAACGTTGTTGCCATTGCTACAGGCATCGTGGTGTCACGC
TCGTCGTTTGGTATGGCTTCATTCAGCTCCGGTTCCCAACGATCAAGGCGAGTTACATGATCCCCCAT
GTTGTGCAAAAAAGCGGTTAGCTCCTTCGGTCCTCCGATCGTTGTCAGAAGTAAGTTGGCCGCAGTGT
TATCACTCATGGTTATGGCAGCACTGCATAATTCTCTTACTGTCATGCCATCCGTAAGATGCTTTTCT
GTGACTGGTGAGTACTCAACCAAGTCATTCTGAGAATAGTGTATGCGGCGACCGAGTTGCTCTTGCCC
GGCGTCAATACGGGATAATACCGCGCCACATAGCAGAACTTTAAAAGTGCTCATCATTGGAAAACGTT
CTTCGGGGCGAAAACTCTCAAGGATCTTACCGCTGTTGAGATCCAGTTCGATGTAACCCACTCGTGCA
CCCAACTGATCTTCAGCATCTTTTACTTTCACCAGCGTTTCTGGGTGAGCAAAAACAGGAAGGCAAAA
TGCCGCAAAAAAGGGAATAAGGGCGACACGGAAATGTTGAATACTCATACTCTTCCTTTTTCAATATT
ATTGAAGCATTTATCAGGGTTATTGTCTCATGAGCGGATACATATTTGAATGTATTTAGAAAAATAAA
CAAATAGGGGTTCCGCGCACATTTCCCCGAAAAGTGCCACCTGACGTCTAAGAAACCATTATTATCAT
GACATTAACCTATAAAAATAGGCGTATCACGAGGCCCTTTCGTCTCGCGCGTTTCGGTGATGACGGTG
AAAACCTCTGACACATGCAGCTCCCGGAGACGGTCACAGCTTGTCTGTAAGCGGATGCCGGGAGCAGA
CAAGCCCGTCAGGGCGCGTCAGCGGGTGTTGGCGGGTGTCGGGGCTGGCTTAACTATGCGGCATCAGA
GCAGATTGTACTGAGAGTGCACCATATGCGGTGTGAAATACCGCACAGATGCGTAAGGAGAAAATACC
GCATCAGGCTAATACGACTCACTATAGGGACCAAGGGGAAAATGAAGTGGTGACTCAAATCATCGAAG
ACCCTCGAGATTACATAGGTCCGGAACCTATGGCCTTCGTGACCGACCTCGAGTCAGAGTAGTTCAAT
AAGGACCTATCAAGTTTGGGCAATTTTTCGTCCCCGACACAAAAATGTCATCCGTGCTTAAAGCATAT
GAGCGATTCACGCTCACTCAAGAACTGCAAGATCAGAGTGAGGAAGGTACAATCCCACCTACAACACT
AAAACCGGTAATCAGGGTATTTATACTAACCTCTAATAACCCAGAGCTAAGATCCCGGCTTCTTCTAT
TCTGCCTACGGATTGTTCTCAGTAATGGTGCAAGGGATTCCCATCGCTTTGGAGCATTACTCACAATG
TTTTCGCTACCATCAGCCACAATGCTCAATCATGTCAAATTAGCTGACCAGTCACCAGAAGCTGATAT
CGAAAGGGTAGAGATCGATGGCTTTGAGGAGGGATCATTCCGCTTAATCCCCAATGCtCGTTCAGGTA
TGAGCCGTGGAGAGATCAATGCCTATGCTGCACTTGCAGAAGATCTACCTGACACACTAAACCATGCA
ACACCTTTCGTTGATTCCGAAGTCGAGGGAACTGCATGGGATGAGATTGAGACTTTCTTAGATATGTG
TTACAGTGTCCTAATGCAGGCATGGATAGTGACTTGCAAGTGCATGACTGCGCCAGACCAACCTGCTG
CTTCTATTGAGAAACGCCTGCAAAAATATCGTCAGCAAGGCAGGATCAACCCGAGATATCTCCTGCAA
CCGGAGGCTCGACGAATAATCCAGAATGTAATCCGGAAGGGAATGGTGGTCAGACATTTCCTCACCTT
TGAACTGCAGCTTGCCCGAGCACAAAGCCTTGTATCAAATAGGTATTATGCTATGGTAGGGGATGTTG
GAAAGTATATAGAGAATTGTGGAATGGGAGGCTTCTTTTTGACACTAAAATATGCATTAGGAACTAGA
TGGCCCACACTTGCTTTAGCTGCATTTTCAGGAGAGCTAACAAAGCTAAAGTCCCTCATGGCATTATA
CCAGACCCTTGGTGAGCAGGCCCGATATTTGGCCCTATTGGAGTCACCACATTTGATGGATTTTGCTG
CAGCAAACTACCCACTGCTATATAGCTATGCTATGGGAATAGGCTATGTGTTAGATGTCAACATGAGG
AACTACGCTTTCTCCAGATCATACATGAACAAGACATATTTCCAATTGGGAATGGAAACTGCAAGAAA
ACAACAGGGTGCAGTTGACATGAGGATGGCAGAAGATCTCGGTCTAACTCAAGCCGAACGCACCGAGA
TGGCAAATACACTTGCCAAATTGACCACAGCAAATCGAGGGGCAGACACCAGGGGAGGAGTCAACCCG
TTCTCATCTGTCACTGGGACAACTCAGGTGCCCGCTGCAGCAACAGGTGACACACTCGAGAGTTACAT
GGCAGCGGATCGACTGAGGCAGAGATATGCTGATGCAGGCACCCATGATGATGAGATGCCACCATTGG
AAGAGGAGGAAGAGGACGACACATCTGCAGGTCCACGCACTGGACCAACTCTTGAACAAGTGGCCTTG
GACATCCAGAACGCAGCAGTTGGAGCTCCCATCCATACAGATGACCTGAATGCCGCACTGGGTGATCT
TGACATCTAGACAATTCAGATCCCAATCTAAAATTGACATACCTAATTGATTAGTTAGATGGAACTAC
AGTGGATTCCATAAGGTTCCTGCCTACCATCGGCTTTAAAGAAAAAAATAGGCCCGGACGGGTTAGCA
ACAAGCGACTGCCGGTGCCAACAGCGCAATCCACAATCTACAATGGATCCCACTGATCTGAGCTTCTC
CCCAGATGAGATCAATAAGCTCATAGAGACAGGCCTGAATACTGTAGAGTATTTTACTTCCCAACAAG
TCACAGGAACATCCTCTCTTGGAAAGAATACAATACCACCAGGGGTCACAGGACTACTAACCAATGCT
GCAGAGGCAAAGATCCAAGAGTCAACTAACCATCAGAAGGGCTCAGTTGGTGGGGGTGCAAAACCAAA
GAAACCGCGACCAAAAATTGCCATTGTGCCAGCAGATGACAAAACAGTGCCCGGAAAGCCGATCCCAA
ACCCTCTATTAGGTCTGGACTCCACCCCGAGCACCCAAACTGTGCTTGATCTAAGTGGGAAAACATTA
CCATCAGGATCCTATAAGGGGGTTAAGCTTGCGAAATTTGGAAAAGAAAATCTGATGACACGGTTCAT
CGAGGAACCCAGAGAGAATCCTATCGCAACCAGTTCCCCCATCGATTTTAAGAGGGGCAGGGATACCG
GCGGGTTCCATAGAAGGGAGTACTCAATCGGATGGGTGGGAGATGAAGTCAAGGTCACTGAGTGGTGC
AATCCATCCTGTTCTCCAATCACCGCTGCAGCAAGGCGATTTGAATGCACTTGTCACCAGTGTCCAGT
CACTTGCTCTGAATGTGAACGAGATACTTAATACAGTGAGAAATTTGGACTCTCGGATGAATCAACTG
GAGACAAAAGTAGATCGCATTCTCTCATCTCAGTCTCTAATCCAGACCATCAAGAATGACATAGTTGG
ACTTAAAGCAGGGATGGCTACTTTAGAAGGAATGATTACAACTGTGAAAATCATGGACCCGGGAGTTC
CCAGTAATGTTACTGTGGAAGATGTACGCAAGACACTAAGTAACCATGCTGTTGTTGTGCCAGAATCA
TTCAATGATAGTTTCTTGACTCAATCTGAAGATGTAATTTCACTTGATGAGTTGGCTCGACCAACTGC
AACAAGTGTTAAGAAGATTGTCAGGAAGGTTCCTCCTCAGAAGGATCTGACTGGATTGAAGATTACAC
TAGAGCAATTGGCAAAGGATTGCATCAGCAAACCGAAGATGAGGGAAGAGTATCTCCTCAAAATCAAC
CAGGCTTCCAGTGAGGCTCAGCTAATTGACCTCAAGAAAGCAATCATCCGCAGTGCAATTTGATCAAG
AAACACCCAATTACACTACACTGGTATGACACTGTACTAACCCTGAGGGTTTTAGAAAAAACGATTAA
CGATAAATAAGCCCGAACACTACACACTACCTGAGGCAGCCATGCCATCCATCAGCATTCCCGCAGAC
CCCACCAATCCACGTCAATCAATAAAAGCGTTCCCAATTGTGATCAACAGTGATGGGGGTGAGAAAGG
CCGCTTGGTTAAACAACTACGCACAACCTACTTGAATGACCTAGATACTCATGAGCCACTGGTGACAT
TCATAAATACCTATGGATTCATCTACGAACAGGATCGGGGGAATACCATTGTCGGAGAGGATCAACTT
GGGAAGAAAAGAGAGGCTGTGACCGCTGCAATGGTTACCCTTGGATGTGGGCCTAATCTACCATCATT
AGGGAATGTCCTGGGACAACTGAGGGAATTCCAGGTCACTGTTAGGAAGACATCCAGCAAAGCGGAAG
AGATGGTCTTTGAAATTGTTAAGTATCCGAGAATATTTCGGGGTCATACATTAATCCAGAAAGGACTA
GTCTGTGTCTCCGCAGAAAAATTTGTTAAGTCACCAGGGAAAATACAATCTGGAATGGACTATCTCTT
CATTCCGACATTTCTGTCAGTGACTTACTGTCCAGCTGCAATCAAATTTCAGGTACCTGGCCCCATGT
TGAAAATGAGATCAAGATACACTCAGAGCTTACAACTTGAACTAATGATAAGAATCCTGTGTAAGCCC
GATTCGCCACTTATGAAGGTCCATACCCCTGACAAGGAGGGAAGAGGATGTCTTGTATCAGTATGGCT
GCATGTATGCAACATCTTCAAATCAGGAAACAAGAATGGCAGTGAGTGGCAGGAATACTGGATGAGAA
AGTGTGCTAACATGCAACTTGAAGTGTCGATTGCAGATATGTGGGGACCAACTATCATAATTCATGCC
AGAGGTCACATTCCCAAAAGTGCTAAGTTGTTTTTTGGAAAGGGTGGATGGAGCTGCCATCCACTTCA
CGAAGTTGTTCCAAGTGTCACTAAAACACTATGGTCCGTGGGCTGTGAGATTACAAAGGCGAAGGCAA
TAATACAAGAGAGTAGCATCTCTCTTCTCGTGGAGACTACTGACATCATAAGTCCAAAAGTCAAAATT
TCATCTAAGCATCGCCGCTTTGqGAAATCAAATTGGGGTCTGTTCAAGAAAACTAAATCACTGCCTAA
CCTGACGGAGCTGGAATGACTGACCTCTAATCGAGACTACACCGCCGCAAACTATAGGTGGGTGGTAC
CTCAGTGATTAATCTTGTAAGCACTGATCGTAGGCTACAACACACTAATATTATCCAGATTAGAGAGC
TTAATTAGCTCTGTATTAATAATAACACTACTATTCCAATAACTGGAATCACCAGCTTGATTTATCTC
CAAAATGATTCAAAGAAAACAAATCATATTAAGACTATCCTAAGGACCGAACCTAGTATTGAAAGAAC
CGTCTCGGTCAATCTAGGTAATCGAGCTGATACCGTCTCGGAAAGCTCAAATCATGCTGCCTGATCCG
GAAGATCCGGAAAGCAAGAAAGCTACAAGGAGAGCAGGAAACCTAATTATCTGCTTCCTATTCATCTT
CTTTCTGTTTGTAACCTTCATTGTTCCAACTCTAAGACACTTGCTGTCCTAACACCTGCTATAGGCTA
TCCACTGCATCATCTCTCCTGCCATACTTCCTACTCACATCATATCTATTTTAAAGAAAAAATAGGCC
CGAACACTAATCGTGCCGGCAGTGCCACTGCACACACAACACTACACATACAATACACTACAATGGTT
GCAGAAGATGCCCCTGTTAGGGCCACTTGCCGAGTATTATTTCGAACAACAACTTTAATCTTTCTATG
CACACTACTAGCATTAAGCATCTCTATCCTTTATGAGAGTTTAATAACCCAAAAGCAAATCATGAGCC
AAGCAGGCTCAACTGGATCTAATTCTGGATTAGGAAGTATCACTGATCTTCTTAATAATATTCTCTCT
GTCGCAAATCAGATTATATATAACTCTGCAGTCGCTCTACCTCTACAATTGGACACTCTTGAATCAAC
ACTCCTTACAGCCATTAAGTCTCTTCAAACCAGTGACAAGCTAGAACAGAACTGCTCGTGGAGTGCTG
CACTGATTAATGATAATAGATACATTAATGGCATCAATCAGTTCTATTTTTCAATTGCTGAGGGTCGC
AATCTGACACTTGGCCCACTTCTTAATATGCCTAGTTTCATTCCAACTGCCACGACACCAGAGGGCTG
CACCAGGATCCCATCATTCTCGCTCACTAAGACACACTGGTGTTATACACACAATGTTATCCTGAATG
GATGCCAGGATCATGTATCCTCAAATCAATTTGTTTCtATGGGAATCATTGAACCCACTTCTGCCGGG
TTTCCATTCTTTCGAACCCTAAAGACTCTATATCTCAGCGATGGGGTCAATCGTAAGAGCTGCTCTAT
CAGTACAGTTCCGGGGGGTTGTATGATGTACTGTTTTGTTTCTACTCAACCAGAGAGGGATGACTACT
TTTCTGCCGCTCCTCCAGAACAACGAATTATTATAATGTACTATAATGATACAATCGTGGAGCGCATA
ATTAATCCACCCGGGGTACTAGATGTATGGGCAACATTGAACCCAGGAACAGGAAGCGGGGTATATTA
TTTAGGTTGGGTGCTCTTTCCAATATATGGCGGCGTGATTAAAGGTACGAGTTTATGGAATAATCAAG
CAAATAAATACTTTATCCCCCAGATGGTTGCTGCTCTCTGCTCACAAAACCAGGCAACTCAAGTCCAA
AATGCTAAGTCATCATACTATAGCAGCTGGTTTGGCAATCGAATGATTCAGTCTGGGATCCTGGCATG
TCCTCTTCGACAGGATCTAACCAATGAGTGTTTAGTTCTGCCCTTTTCTAATGATCAGGTGCTTATGG
GTGCTGAAGGGAGATTATACATGTATGGTGACTCGGTGTATTACTATCAAAGAAGCAATAGTTGGTGG
CCTATGACCATGCTGTATAAGGTAACCATAACATTCACTAATGGTCAGCCATCTGCTATATCAGCTCA
GAATGTGCCCACACAGCAGGTCCCTAGACCTGGGACAGGAGACTGCTCTGCAACCAATAGATGTCCCG
GTTTTTGCTTGACAGGAGTGTATGCCGATGCCTGGTTACTGACCAACCCTTCGTCTACCAGTACATTT
GGATCAGAAGCAACCTTCACTGGTTCTTATCTCAACACAGCAACTCAGCGTATCAATCCGACGATGTA
TATCGCGAACAACACACAGATCATAAGCTCACAGCAATTTGGATCAAGCGGTCAAGAAGCAGCATATG
GCCACACAACtTGTTTTAGGGACACAGGCTCTGTTATGGTATACTGTATCTATATTATTGAATTGTCC
TCATCTCTCTTAGGACAATTTCAGATTGTCCCATTTATCCGTCAGGTGACACTATCCTAAAGGCAGAA
GCCTTCAGGTCTGACCCAGCCAATCAAAGCATTATACCAGACCATGGCCTACCATCGGCTTTAAAGAA
AAAAATAGGCCCGGACGGGTTAGCAACAAGCGGCGGCCGCAATGGTGAGCAAGGGCGAGGAGGATAAC
ATGGCCATCATCAAGGAGTTCATGCGCTTCAAGGTGCACATGGAGGGCTCCGTGAACGGCCACGAGTT
CGAGATCGAGGGCGAGGGCGAGGGCCGCCCCTACGAGGGCACCCAGACCGCCAAGCTGAAGGTGACCA
AGGGTGGCCCCCTGCCCTTCGCCTGGGACATCCTGTCCCCTCAGTTCATGTACGGCTCCAAGGCCTAC
GTGAAGCACCCCGCCGACATCCCCGACTACTTGAAGCTGTCCTTCCCCGAGGGCTTCAAGTGGGAGCG
CGTGATGAACTTCGAGGACGGCGGCGTGGTGACCGTGACCCAGGACTCCTCCCTGCAGGACGGCGAGT
TCATCTACAAGGTGAAGCTGCGCGGCACCAACTTCCCCTCCGACGGCCCCGTAATGCAGAAGAAGACC
ATGGGCTGGGAGGCCTCCTCCGAGCGGATGTACCCCGAGGACGGCGCCCTGAAGGGCGAGATCAAGCA
GAGGCTGAAGCTGAAGGACGGCGGCCACTACGACGCTGAGGTCAAGACCACCTACAAGGCCAAGAAGC
CCGTGCAGCTGCCCGGCGCCTACAACGTCAACATCAAGTTGGACATCACCTCCCACAACGAGGACTAC
ACCATCGTGGAACAGTACGAACGCGCCGAGGGCCGCCACTCCACCGGCGGCATGGACGAGCTGTACAA
GTAGGCGGCCGCCTAAGGTCGACTCATGGAATGCATACCAAACATTATTGACACTAATGACACACAAA
ATTGGTTTTAAGAAAAACCAAGAGAACAATAGGCCAGAATGGCTGGGTCTCGGGAGATATTACTCCCT
GAAGTCCATCTCAATTCACCAATTGTAAAGCATAAGCTATACTATTACATTCTACTTGGAAACCTCCC
AAATGAGATCGACCTTGACGATTTAGGTCCATTACATAATCAAAATTGGAATCAGATAGCACATGAAG
AGTCTAACTTAGCTCAACGCTTGGTAAATGTAAGAAATTTTCTAATTACCCACATCCCTGATCTTAGA
AAGGGCCATTGGCAAGAGTATGTCAATGTAATACTGTGGCCGCGAATTCTTCCCTTGATCCCGGATTT
TAAAATCAATGACCAATTGCCTCTGCTCAAAAATTGGGACAAGTTAGTTAAAGAATCATGTTCAGTAA
TCAATGCAGGTACTTCCCAGTGCATTCAGAATCTCAGCTATGGACTGACAGGTCGTGGGAACCTCTTT
ACACGATCACGTGAACTCTCTGGTGACCGCAGGGATATTGATCTTAAGACAGTTGTGGCAGCATGGCA
TGACTCAGACTGGAAAAGAATAAGTGATTTTTGGATTATGATCAAATTCCAGATGAGACAATTAATTG
TTAGGCAAACAGATCATAATGATTCTGATTTAATCACGTATATCGAAAATAGAGAAGGCATAATCATC
ATAACCCCTGAACTGGTAGCATTATTTAACACTGAGAATCATACACTAACATACATGACCTTTGAAAT
TGTACTGATGGTTTCAGATATGTACGAAGGTCGTCACAACATTTTATCACTATGCACAGTTAGCACTT
ACCTGAATCCTCTGAAGAAAAGAATAACATATTTATTGAGCCTTGTAGATAACTTAGCTTTTCAGATA
GGTGATGCTGTATATAACATAATTGCTTTGCTAGAATCCTTTGTATATGCACAGTTGCAAATGTCAGA
TCCCATCCCAGAACTCAGAGGACAATTCCATGCATTCGTATGTTCTGAGATTCTTGATGCACTAAGAG
GAACTAATAGTTTCACCCAGGATGAATTAAGAACTGTGACAACTAATTTGATATCCCCATTCCAAGAT
CTGACCCCAGATCTTACGGCTGAATTGCTCTGTATAATGAGGCTTTGGGGACACCCCATGCTCACTGC
CAGTCAAGCTGCAGGAAAGGTACGCGAGTCTATGTGTGCTGGAAAAGTATTAGACTTTCCCACCATTA
TGAAAACACTAGCCTTTTTCCATACTATTCTGATCAATGGATACAGGAGGAAGCATCATGGAGTATGG
CCACCCTTAAACTTACCGGGTAATGCTTCAAAGGGTCTCACGGAACTTATGAATGACAATACTGAAAT
AAGCTATGAATTCACACTTAAGCATTGGAAGGAAGTCTCTCTTATAAAATTCAAGAAATGTTTTGATG
CAGACGCAGGTGAGGAACTCAGTATATTTATGAAAGATAAGGCAATTAGTGCCCCAAAACAAGACTGG
ATGAGTGTGTTTAGAAGAAGCCTAATCAAACAGCGCCATCAGCATCATCAGGTCCCCCTACCAAATCC
ATTCAATCGACGGCTGTTGCTAAACTTTCTCGGAGATGACAAATTCGACCCGAATGTGGAGCTACAGT
ATGTAACATCAGGTGAGTATCTACATGATGACACGTTTTGTGCATCATATTCACTAAAAGAGAAGGAA
ATTAAACCTGATGGTCGAATTTTTGCAAAGTTGACTAAGAGAATGAGATCATGTCAAGTTATAGCAGA
ATCTCTTTTAGCGAACCATGCTGGGAAGTTAATGAAAGAGAATGGTGTTGTGATGAATCAGCTATCAT
TAACAAAATCACTATTAACAATGAGTCAGATTGGAATAATATCCGAGAAAGCTAGAAAGTCAACTCGA
GATAACATAAATCAACCTGGTTTCCAGAATATCCAGAGAAATAAATCACATCACTCCAAGCAAGTCAA
TCAGCGAGATCCAAGTGATGACTTTGAATTGGCAGCATCTTTTTTAACTACTGATCTCAAAAAATATT
GTTTACAATGGAGGTACCAGACAATTATCCCATTTGCTCAATCATTAAACAGAATGTATGGTTATCCT
CATCTCTTTGAGTGGATTCACTTACGGCTAATGCGTAGTACACTTTACGTGGGGGATCCCTTCAACCC
ACCAGCAGATACCAGTCAATTTGATCTAGATAAAGTAATTAATGGAGATATCTTCATTGTATCACCCA
GAGGTGGAATTGAAGGGCTGTGTCAAAAGGCTTGGACAATGATATCTATCGCTGTGATAATTCTATCT
GCCACAGAGTCTGGCACACGAGTAATGAGTATGGTGCAGGGAGATAATCAAGCAATTGCTGTCACCAC
ACGAGTACCAAGGAGCCTGCCGACTCTTGAGAAAAAGACTATTGCTTTTAGATCTTGTAATCTATTCT
TTGAGAGGTTAAAATGTAATAATTTTGGATTAGGTCACCATTTGAAAGAACAAGAGACTATCATTAGT
TCTCACTTCTTTGTTTATAGCAAGAGAATATTCTATCAGGGGAGGATTCTAACGCAAGCCTTAAAAAA
TGCTAGTAAGCTCTGCTTGACAGCTGATGTCCTAGGAGAATGCACCCAATCATCATGTTCTAATCTTG
CAACTACTGTCATGAGGTTAACTGAGAATGGTGTTGAAAAAGATATCTGTTTCTACTTGAATATCTAT
ATGACCATCAAACAGCTCTCCTATGATATCATCTTCCCTCAAGTGTCAATTCCTGGAGATCAGATCAC
ATTAGAATACATAAATAATCCACACCTGGTATCACGATTGGCTCTTTTGCCATCCCAGTTAGGAGGTC
TAAACTACCTGTCATGCAGTAGGCTGTTCAATCGAAACATAGGCGACCCGGTGGTTTCCGCAGTTGCA
GATCTTAAGAGATTAATTAAATCAGGATGTATGGATTACTGGATCCTTTATAACTTATTAGGGAGAAA
ACCGGGAAACGGCTCATGGGCTACTTTAGCAGCTGACCCGTACTCAATCAATATAGAGTATCAATACC
CTCCAACTACAGCTCTTAAGAGGCACACCCAACAAGCTCTGATGGAACTCAGTACGAATCCAATGTTA
CGTGGCATATTCTCTGACAATGCACAGGCAGAAGAAAATAACCTTGCTAGGTTTCTCCTGGATAGGGA
GGTGATCTTTCCGCGTGTAGCTCACATCATCATTGAGCAAACCAGTGTCGGGAGGAGAAAACAGATTC
AAGGATATTTGGATTCAACTAGATCGATAATGAGGAAATCACTAGAAATTAAGCCCTTATCCAATAGG
AAGCTTAATGAAATACTGGATTACAACATCAATTACCTAGCTTACAATTTGGCATTACTCAAGAATGC
TATTGAACCTCCGACTTATTTGAAGGCAATGACACTTGAAACATGTAGCATCGACATTGCAAGGAACC
TCCGGAAGCTCTCCTGGGCCCCACTCTTGGGTGGGAGAAATCTTGAAGGATTAGAGACGCCAGATCCC
ATTGAAATTACTGCAGGAGCATTAATTGTTGGATCGGGCTACTGTGAACAGTGTGCTGCAGGAGACAA
TCGATTCACATGGTTTTTCTTGCCATCTGGTATCGAGATAGGAGGGGATCCCCGTGATAATCCTCCTA
TCCGTGTACCGTACATTGGCTCCAGGACTGATGAGAGGAGGGTAGCCTCAATGGCATACATCAGGGGT
GCCTCGAGTAGCTTAAAAGCAGTTCTTAGACTGGCGGGAGTGTACATCTGGGCATTCGGAGATACTCT
GGAGAATTGGATAGATGCACTGGATTTGTCTCACACTAGAGTTAACATCACACTTGAACAGCTGCAAT
CCCTCACCCCACTTCCAACCTCTGCCAATCTAACCCATCGGTTGGATGATGGCACAACTACCCTAAAG
TTTACTCCTGCGAGCTCTTACACCTTTTCAAGTTTCACTCATATATCAAATGATGAGCAATACCTGAC
AATTAATGACAAAACTGCAGATTCAAATATAATCTACCAACAGTTAATGATCACTGGACTCGGAATCT
TAGAAACATGGAATAATCCCCCAATCAATAGAACATTCGAAGAATCTACCCTACATTTGCACACTGGT
GCATCATGTTGTGTCCGACCTGTGGACTCCTGCATTCTCTCAGAAGCATTAACAGTCAAGCCACATAT
TACAGTACCGTACAGCAATAAATTTGTATTTGATGAGGACCCGCTATCTGAATATGAAACTGCAAAAC
TGGAATCGTTATCATTCCAAGCCCAATTAGGCAACATTGATGCTGTAGATATGACAGGTAAATTAACA
TTATTGTCCCAATTCACTGCAAGGCAGATTATCAATGCAATCACTGGACTCGATGAGTCTGTCTCTCT
TACTAATGATGCCATTGTTGCATCAGACTATGTCTCCAATTGGATTAGTGAATGCATGTATACCAAAT
TAGATGAATTATTTATGTATTGTGGGTGGGAACTACTATTGGAACTATCCTATCAAATGTATTATCTG
AGGGTAGTTGGGTGGAGTAATATAGTGGATTATTCTTACATGATCTTGAGAAGAATCCCGGGTGCAGC
ATTAAACAATCTGGCATCTACATTAAGTCATCCAAAACTTTTCCGACGAGCTATCAACCTAGATATAG
TTGCCCCCTTAAATGCTCCTCATTTTGCATCTCTGGACTACATCAAGATGAGTGTGGATGCAATACTC
TGGGGCTGTAAAAGAGTCATCAATGTGCTCTCCAATGGAGGGGACTTAGAATTAGTTGTGACATCTGA
AGATAGCCTTATTCTCAGTGACCGATCCATGAATCTCATTGCAAGGAAATTAACTTTATTATCACTGA
TTCACCATAATGGTTTGGAACTACCAAAGATTAAGGGGTTCTCTCCTGATGAGAAGTGTTTCGCTTTG
ACAGAATTTTTGAGGAAAGTGGTGAACTCAGGGTTGAGTTCAATAGAGAACCTATCAAATTTTATGTA
CAATGTGGAGAACCCACGGCTTGCAGCATTCGCCAGCAACAATTACTACCTGACCAGAAAATTATTGA
ATTCAATACGAGATACTGAGTCGGGTCAAGTAGCAGTCACCTCATATTATGAATCATTAGAATATATT
GATAGTCTTAAGCTAACCCCACATGTGCCTGGCACCTCATGCATTGAGGATGATAGTCTATGTACAAA
TGATTACATAATCTGGATCATAGAGTCTAATGCAAACTTGGAGAAGTATCCAATTCCAAATAGCCCTG
AGGATGATTCCAATTTCCATAACTTTAAGTTGAATGCTCCATCGCACCATACCTTACGCCCATTAGGG
TTGTCATCAACTGCTTGGTATAAGGGTATAAGCTGCTGCAGGTACCTTGAGCGATTAAAGCTACCACA
AGGTGATCATTTATATATTGgAGAAGGTAGTGGTGCCAGTATGACAATCATAGAATACCTATTCCCAG
GAAGAAAGATATATTACAATTCTTTATTTAGTAGTGGTGACAATCCCCCACAAAGAAATTATGCACCA
ATGCCTACTCAGTTCATTGAGAGTGTCCCATACAAGCTCTGGCAAGCACACACAGATCAATATCCCGA
GATTTTTGAGGACTTCATCCCTCTATGGAACGGAAACGCCGCCATGACTGACATAGGAATGACAGCTT
GTGTAGAATTCATCATCAATCGAGTCGGCCCAAGGACTTGCAGTTTAGTACATGTAGATTTGGAATCA
AGTGCAAGCTTAAATCAACAATGCCTGTCAAAGCCGATAATTAATGCTATCATCACTGCTACAACTGT
TTTGTGCCCTCATGGGGTGCTTATTCTGAAATATAGTTGGTTGCCATTTACTAGATTTAGTACTTTGA
TCACTTTCTTATGGTGCTACTTTGAGAGAATCACTGTTCTTAGGAGCACATATTCTGATCCAGCTAAT
CATGAGGTTTATTTAATTTGTATCCTTGCCAACAACTTTGCATTCCAGACTGTCTCGCAGGCAACAGG
AATGGCGATGACTTTAACTGATCAAGGGTTTACTTTGATATCACCTGAAAGAATAAATCAGTATTGGG
ATGGTCACTTGAAGCAAGAACGTATCGTAGCAGAAGCAATTGATAAGGTGGTTCTAGGAGAAAATGCT
CTATTTAATTCGAGTGATAATGAATTAATTCTCAAATGTGGAGGGACACCAAATGCACGGAATCTCAT
CGATATCGAGCCAGTCGCAACTTTCATAGAATTTGAACAATTGATCTGCACAATGTTGACAACCCACT
TGAAGGAAATAATTGATATAACAAGGTCTGGAACCCAGGATTATGAAAGTTTATTACTCACTCCTTAC
AATTTAGGTCTTCTTGGTAAAATCAGTACGATAGTGAGATTATTAACAGAAAGGATTCTAAATCATAC
TATCAGGAATTGGTTGATCCTCCCACCTTCGCTCCGGATGATCGTGAAGCAGGACTTGGAATTCGGCA
TATTCAGGATTACTTCCATCCTCAATTCTGATCGGTTCCTGAAGCTTTCTCCAAATAGGAAATACTTG
ATTGCACAATTAACTGCAGGCTACATTAGGAAATTGATTGAGGGGGATTGCAATATCGATCTAACCAG
ACCTATCCAAAAGCAAATCTGGAAAGCATTAGGTTGTGTAGTCTATTGTCACGATCCAATGGATCAAA
GGGAGTCAACAGAGTTTATTGATATAAATATTAATGAAGAAATAGACCGCGGGATCGATGGCGAGGAA
ATCTAAACATATCAAGAATCAGAATTAGTTTAAGAAAAAAGAAGAGGATTAATCTTGGTTTTCCCCTT
GGTGGGTCGGCATGGCATCTCCACCTCCTCGCGGTCCGACCTGGGCATCCGAAGGAGGACGCACGTCC
ACTCGGATGGCTAAGGGAGCGGCCGGGGATCCGGCTGCTAACAAAGCCCGAAAGGAAGCTGAGTTGGC
TGCTGCCACCGCTGAGCAATAACTAGCATAACCCCTTGGGGCCTCTAAACGGGTCTTGAGGGGTTTTT
TGCTGAAAGGAGGAACTATATCCGGAT
PIV5 deltaF vector expressing hu anti V5 mAB- and mCherry
pBH276.P[D100/F157]ΔF-V5hAb-mCherry
CATGTGAGCAAAAGGCCAGCAAAAGGCCAGGAACCGTAAAAAGGCCGCGTTGCTGGCGTTTTTCCATA
GGCTCCGCCCCCCTGACGAGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCCTGTTCCGACCCTGCCGCT
TACCGGATACCTGTCCGCCTTTCTCCCTTCGGGAAGCGTGGCGCTTTCTCATAGCTCACGCTGTAGGT
ATCTCAGTTCGGTGTAGGTCGTTCGCTCCAAGCTGGGCTGTGTGCACGAACCCCCCGTTCAGCCCGAC
CGCTGCGCCTTATCCGGTAACTATCGTCTTGAGTCCAACCCGGTAAGACACGACTTATCGCCACTGGC
AGCAGCCACTGGTAACAGGATTAGCAGAGCGAGGTATGTAGGCGGTGCTACAGAGTTCTTGAAGTGGT
GGCCTAACTACGGCTACACTAGAAGAACAGTATTTGGTATCTGCGCTCTGCTGAAGCCAGTTACCTTC
GGAAAAAGAGTTGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTTCTACGGGGTCTG
ACGCTCAGTGGAACGAAAACTCACGTTAAGGGATTTTGGTCATGAGATTATCAAAAAGGATCTTCACC
TAGATCCTTTTAAATTAAAAATGAAGTTTTAAATCAATCTAAAGTATATATGAGTAAACTTGGTCTGA
CAGTTACCAATGCTTAATCAGTGAGGCACCTATCTCAGCGATCTGTCTATTTCGTTCATCCATAGTTG
CCTGACTCCCCGTCGTGTAGATAACTACGATACGGGAGGGCTTACCATCTGGCCCCAGTGCTGCAATG
ATACCGCGAGACCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGGGAAGCTAGAG
TAAGTAGTTCGCCAGTTAATAGTTTGCGCAACGTTGTTGCCATTGCTACAGGCATCGTGGTGTCACGC
TCGTCGTTTGGTATGGCTTCATTCAGCTCCGGTTCCCAACGATCAAGGCGAGTTACATGATCCCCCAT
GTTGTGCAAAAAAGCGGTTAGCTCCTTCGGTCCTCCGATCGTTGTCAGAAGTAAGTTGGCCGCAGTGT
TATCACTCATGGTTATGGCAGCACTGCATAATTCTCTTACTGTCATGCCATCCGTAAGATGCTTTTCT
GTGACTGGTGAGTACTCAACCAAGTCATTCTGAGAATAGTGTATGCGGCGACCGAGTTGCTCTTGCCC
GGCGTCAATACGGGATAATACCGCGCCACATAGCAGAACTTTAAAAGTGCTCATCATTGGAAAACGTT
CTTCGGGGCGAAAACTCTCAAGGATCTTACCGCTGTTGAGATCCAGTTCGATGTAACCCACTCGTGCA
CCCAACTGATCTTCAGCATCTTTTACTTTCACCAGCGTTTCTGGGTGAGCAAAAACAGGAAGGCAAAA
TGCCGCAAAAAAGGGAATAAGGGCGACACGGAAATGTTGAATACTCATACTCTTCCTTTTTCAATATT
ATTGAAGCATTTATCAGGGTTATTGTCTCATGAGCGGATACATATTTGAATGTATTTAGAAAAATAAA
CAAATAGGGGTTCCGCGCACATTTCCCCGAAAAGTGCCACCTGACGTCTAAGAAACCATTATTATCAT
GACATTAACCTATAAAAATAGGCGTATCACGAGGCCCTTTCGTCTCGCGCGTTTCGGTGATGACGGTG
AAAACCTCTGACACATGCAGCTCCCGGAGACGGTCACAGCTTGTCTGTAAGCGGATGCCGGGAGCAGA
CAAGCCCGTCAGGGCGCGTCAGCGGGTGTTGGCGGGTGTCGGGGCTGGCTTAACTATGCGGCATCAGA
GCAGATTGTACTGAGAGTGCACCATATGCGGTGTGAAATACCGCACAGATGCGTAAGGAGAAAATACC
GCATCAGGCTAATACGACTCACTATAGGGACCAAGGGGAAAATGAAGTGGTGACTCAAATCATCGAAG
ACCCTCGAGATTACATAGGTCCGGAACCTATGGCCTTCGTGACCGACCTCGAGTCAGAGTAGTTCAAT
AAGGACCTATCAAGTTTGGGCAATTTTTCGTCCCCGACACAAAAATGTCATCCGTGCTTAAAGCATAT
GAGCGATTCACGCTCACTCAAGAACTGCAAGATCAGAGTGAGGAAGGTACAATCCCACCTACAACACT
AAAACCGGTAATCAGGGTATTTATACTAACCTCTAATAACCCAGAGCTAAGATCCCGGCTTCTTCTAT
TCTGCCTACGGATTGTTCTCAGTAATGGTGCAAGGGATTCCCATCGCTTTGGAGCATTACTCACAATG
TTTTCGCTACCATCAGCCACAATGCTCAATCATGTCAAATTAGCTGACCAGTCACCAGAAGCTGATAT
CGAAAGGGTAGAGATCGATGGCTTTGAGGAGGGATCATTCCGCTTAATCCCCAATGCtCGTTCAGGTA
TGAGCCGTGGAGAGATCAATGCCTATGCTGCACTTGCAGAAGATCTACCTGACACACTAAACCATGCA
ACACCTTTCGTTGATTCCGAAGTCGAGGGAACTGCATGGGATGAGATTGAGACTTTCTTAGATATGTG
TTACAGTGTCCTAATGCAGGCATGGATAGTGACTTGCAAGTGCATGACTGCGCCAGACCAACCTGCTG
CTTCTATTGAGAAACGCCTGCAAAAATATCGTCAGCAAGGCAGGATCAACCCGAGATATCTCCTGCAA
CCGGAGGCTCGACGAATAATCCAGAATGTAATCCGGAAGGGAATGGTGGTCAGACATTTCCTCACCTT
TGAACTGCAGCTTGCCCGAGCACAAAGCCTTGTATCAAATAGGTATTATGCTATGGTAGGGGATGTTG
GAAAGTATATAGAGAATTGTGGAATGGGAGGCTTCTTTTTGACACTAAAATATGCATTAGGAACTAGA
TGGCCCACACTTGCTTTAGCTGCATTTTCAGGAGAGCTAACAAAGCTAAAGTCCCTCATGGCATTATA
CCAGACCCTTGGTGAGCAGGCCCGATATTTGGCCCTATTGGAGTCACCACATTTGATGGATTTTGCTG
CAGCAAACTACCCACTGCTATATAGCTATGCTATGGGAATAGGCTATGTGTTAGATGTCAACATGAGG
AACTACGCTTTCTCCAGATCATACATGAACAAGACATATTTCCAATTGGGAATGGAAACTGCAAGAAA
ACAACAGGGTGCAGTTGACATGAGGATGGCAGAAGATCTCGGTCTAACTCAAGCCGAACGCACCGAGA
TGGCAAATACACTTGCCAAATTGACCACAGCAAATCGAGGGGCAGACACCAGGGGAGGAGTCAACCCG
TTCTCATCTGTCACTGGGACAACTCAGGTGCCCGCTGCAGCAACAGGTGACACACTCGAGAGTTACAT
GGCAGCGGATCGACTGAGGCAGAGATATGCTGATGCAGGCACCCATGATGATGAGATGCCACCATTGG
AAGAGGAGGAAGAGGACGACACATCTGCAGGTCCACGCACTGGACCAACTCTTGAACAAGTGGCCTTG
GACATCCAGAACGCAGCAGTTGGAGCTCCCATCCATACAGATGACCTGAATGCCGCACTGGGTGATCT
TGACATCTAGACAATTCAGATCCCAATCTAAAATTGACATACCTAATTGATTAGTTAGATGGAACTAC
AGTGGATTCCATAAGGTTCCTGCCTACCATCGGCTTTAAAGAAAAAAATAGGCCCGGACGGGTTAGCA
ACAAGCGACTGCCGGTGCCAACAGCGCAATCCACAATCTACAATGGATCCCACTGATCTGAGCTTCTC
CCCAGATGAGATCAATAAGCTCATAGAGACAGGCCTGAATACTGTAGAGTATTTTACTTCCCAACAAG
TCACAGGAACATCCTCTCTTGGAAAGAATACAATACCACCAGGGGTCACAGGACTACTAACCAATGCT
GCAGAGGCAAAGATCCAAGAGTCAACTAACCATCAGAAGGGCTCAGTTGGTGGGGGTGCAAAACCAAA
GAAACCGCGACCAAAAATTGCCATTGTGCCAGCAGATGACAAAACAGTGCCCGGAAAGCCGATCCCAG
ACCCTCTATTAGGTCTGGACTCCACCCCGAGCACCCAAACTGTGCTTGATCTAAGTGGGAAAACATTA
CCATCAGGATCCTATAAGGGGGTTAAGCTTGCGAAATTTGGAAAAGAAAATCTGATGACACGGTTCAT
CGAGGAACCCAGAGAGAATCCTATCGCAACCAGTTTCCCCATCGATTTTAAGAGGGGCAGGTATACCG
GCGGGTTCCATAGAAGGGAGTACTCAATCGGATGGGTGGGAGATGAAGTCAAGGTCACTGAGTGGTGC
AATCCATCCTGTTCTCCAATCACCGCTGCAGCAAGGCGATTTGAATGCACTTGTCACCAGTGTCCAGT
CACTTGCTCTGAATGTGAACGAGATACTTAATACAGTGAGAAATTTGGACTCTCGGATGAATCAACTG
GAGACAAAAGTAGATCGCATTCTCTCATCTCAGTCTCTAATCCAGACCATCAAGAATGACATAGTTGG
ACTTAAAGCAGGGATGGCTACTTTAGAAGGAATGATTACAACTGTGAAAATCATGGACCCGGGAGTTC
CCAGTAATGTTACTGTGGAAGATGTACGCAAGACACTAAGTAACCATGCTGTTGTTGTGCCAGAATCA
TTCAATGATAGTTTCTTGACTCAATCTGAAGATGTAATTTCACTTGATGAGTTGGCTCGACCAACTGC
AACAAGTGTTAAGAAGATTGTCAGGAAGGTTCCTCCTCAGAAGGATCTGACTGGATTGAAGATTACAC
TAGAGCAATTGGCAAAGGATTGCATCAGCAAACCGAAGATGAGGGAAGAGTATCTCCTCAAAATCAAC
CAGGCTTCCAGTGAGGCTCAGCTAATTGACCTCAAGAAAGCAATCATCCGCAGTGCAATTTGATCAAG
AAACACCCAATTACACTACACTGGTATGACACTGTACTAACCCTGAGGGTTTTAGAAAAAACGATTAA
CGATAAATAAGCCCGAACACTACACACTACCTGAGGCAGCCATGCCATCCATCAGCATTCCCGCAGAC
CCCACCAATCCACGTCAATCAATAAAAGCGTTCCCAATTGTGATCAACAGTGATGGGGGTGAGAAAGG
CCGCTTGGTTAAACAACTACGCACAACCTACTTGAATGACCTAGATACTCATGAGCCACTGGTGACAT
TCATAAATACCTATGGATTCATCTACGAACAGGATCGGGGGAATACCATTGTCGGAGAGGATCAACTT
GGGAAGAAAAGAGAGGCTGTGACCGCTGCAATGGTTACCCTTGGATGTGGGCCTAATCTACCATCATT
AGGGAATGTCCTGGGACAACTGAGGGAATTCCAGGTCACTGTTAGGAAGACATCCAGCAAAGCGGAAG
AGATGGTCTTTGAAATTGTTAAGTATCCGAGAATATTTCGGGGTCATACATTAATCCAGAAAGGACTA
GTCTGTGTCTCCGCAGAAAAATTTGTTAAGTCACCAGGGAAAATACAATCTGGAATGGACTATCTCTT
CATTCCGACATTTCTGTCAGTGACTTACTGTCCAGCTGCAATCAAATTTCAGGTACCTGGCCCCATGT
TGAAAATGAGATCAAGATACACTCAGAGCTTACAACTTGAACTAATGATAAGAATCCTGTGTAAGCCC
GATTCGCCACTTATGAAGGTCCATACCCCTGACAAGGAGGGAAGAGGATGTCTTGTATCAGTATGGCT
GCATGTATGCAACATCTTCAAATCAGGAAACAAGAATGGCAGTGAGTGGCAGGAATACTGGATGAGAA
AGTGTGCTAACATGCAACTTGAAGTGTCGATTGCAGATATGTGGGGACCAACTATCATAATTCATGCC
AGAGGTCACATTCCCAAAAGTGCTAAGTTGTTTTTTGGAAAGGGTGGATGGAGCTGCCATCCACTTCA
CGAAGTTGTTCCAAGTGTCACTAAAACACTATGGTCCGTGGGCTGTGAGATTACAAAGGCGAAGGCAA
TAATACAAGAGAGTAGCATCTCTCTTCTCGTGGAGACTACTGACATCATAAGTCCAAAAGTCAAAATT
TCATCTAAGCATCGCCGCTTTGqGAAATCAAATTGGGGTCTGTTCAAGAAAACTAAATCACTGCCTAA
CCTGACGGAGCTGGAATGACTGACCTCTAATCGAGACTACACCGCCGCAAACTATAGGTGGGTGGTAC
CTCAGTGATTAATCTTGTAAGCACTGATCGTAGGCTACAACACACTAATATTATCCAGATTAGAGAGC
TTAATTAGCTCTGTATTAATAATAACACTACTATTCCAATAACTGGAATCACCAGCTTGATTTATCTC
CAAAATGATTCAAAGAAAACAAATCATATTAAGACTATCCTAAGCACGAACCCATATCGTCCTTCAAA
TCATGGACCCCAAGGGCAGCCTGAGCTGGAGAATCCTGCTGTTCCTGAGCCTGGCCTTCGAGCTGAGC
TACGGCGACGTGCAGGTGGTGGAGAGCGGAGGCGGAGTGGTGCAGCCAGGCGGCAGCCTGAGACTGAG
CTGCGCTGCGAGTGGCTTCACCTTCAGCAGCTTCGGgATGCACTGGGTGAGGCAGGCACCCGGCAAGG
GCCTGGAGTGGGTGGCCTACATCAACACCGACAGCACCACCATCTACTACGCAGACAGCGTGAAGGGC
AGGTTCACCATCAGCAGGGACAACAGCAAGAACACCCTGTACCTGCAGATGAACTCTTTGCGGGCAGA
AGACACTGCCGTGTACTACTGCGCCAGTGCTGGCCCCTACTATGGCTTTGACTACTGGGGACAGGGCA
CCCTGGTGACCGTGAGCAGCGCTAGCACCAAGGGCCtCAGCGTGTTCCCTCTGGCCCCCAGCAGCAAG
AGCACCAGCGGCGGAACCGCCGCCCTGGGCTGCCTGGTGAAGGACTACTTCCCCGAGCCCGTGACCGT
GTCCTGGAACAGCGGCGCTCTGACCAGCGGAGTGCACACCTTCCCTGCCGTGCTGCAGAGCAGCGGCC
TGTACTCCCTGAGCAGCGTGGTGACCGTGCCCAGCAGCAGCCTGGGCACCCAGACCTACATCTGCAAC
GTGAACCACAAGCCCTCCAACACCAAGGTGGACAAGAAGGTGGAGCCTAAGAGCTGCGACAAGACCCA
CACCTGCCCTCCCTGCCCCGCCCCCGAGCTGCTGGGCGGACCCAGCGTGTTCCTGTTCCCTCCCAAGC
CCAAGGACACCCTGATGATCAGCCGCACCCCCGAGGTGACCTGCGTGGTGGTGGACGTGAGCCACGAG
GACCCCGAGGTGAAGTTCAACTGGTACGTGGACGGCGTGGAGGTGCACAACGCCAAGACCAAGCCTCG
GGAGGAGCAGTACAACTCCACCTACCGCGTGGTGAGCGTGCTGACCGTGCTGCACCAGGACTGGCTGA
ACGGCAAGGAGTACAAGTGCAAGGTGAGCAACAAGGCCCTGCCCGCTCCCATCGAGAAGACCATCAGC
AAGGCCAAGGGCCAGCCtCGGGAGCCTCAGGTGTACACCCTGCCCCCCAGCCGCGAAGAGATGACCAA
GAACCAGGTGAGCCTGACCTGCCTGGTGAAGGGCTTCTACCCCTCCGACATCGCCGTGGAGTGGGAGA
GCAACGGCCAGCCTGAGAACAACTACAAGACCACCCCTCCCGTGCTGGACAGCGACGGCAGCTTCTTC
CTGTACAGCAAGCTGACCGTGGACAAGTCCCGGTGGCAGCAGGGCAACGTGTTCAGCTGCAGCGTGAT
GCACGAGGCCCTGCACAACCACTACACCCAGAAGAGCCTGAGCCTGAGCCCCGGACGCGCAAAAAGAt
ctagaGAGGGCAGGGGAAGTCTTCTAACATGCGGGGACGTGGAGGAAAATCCCGGGCCtATGGAGACC
GACACCCTGCTGCTCTGGGTGCTGCTGCTCTGGGTGCCCGGCTCCACCGGAGACATCGTGATGACCCA
GAGTCCACTGAGCCTGCCCGTGACCCCCGGTGAGCCAGCCAGCATCAGCTGCAGGAGCAGCAAGAGCC
TGGTGCACAGCAACGGCATCACCTACCTGTACTGGTACGTGCAGAAGCCCGGACAGAGCCCCCAGTTG
CTCATTTATCAGATGAGCAGCCTGGCAAGCGGCGTGCCCGACAGGTTCAGCGGCTCCGGCAGCGGCAC
CGACTTCAGCCTGAAGATCAGCCGGGTGGAGGCCGAGGACGTGGGCGTGTACTACTGCGGCCAAATTC
TGGAGCTGCCCTTCACCTTCGGCCAGGGAACCAAGGTGGAGATCAAGCGGACCGTGGCCGCCCCCAGC
GTGTTCATCTTCCCTCCCAGCGACGAGCAGCTGAAGTCTGGCACCGCCAGCGTGGTGTGCCTGCTGAA
CAACTTCTACCCCCGCGAGGCCAAGGTGCAGTGGAAGGTGGACAACGCCCTGCAGAGCGGCAACAGCC
AGGAGAGCGTGACCGAGCAGGACTCCAAGGACAGCACCTACAGCCTGAGCAGCACCCTGACCCTGAGC
AAGGCCGACTACGAGAAGCACAAGGTGTACGCCTGCGAGGTGACCCACCAGGGACTGTCTAGCCCCGT
GACCAAGAGCTTCAACCGGGGCGAGTGCTAATGAGCATTCCACCACTCACGATCTGATCTCAGTGAGA
AAAATCAACCTGCAACTCTTGGAACAAGATAAGACAGTCATCCATTAGTAATTTTTAAGAAAAAAACG
ATAGGACCGAACCTagTATTGAAAGAACCGTCTCGGTCAATCTAGGTAATCGAGCTGATACCGTCTCG
GAAAGCTCAAATCATGCTGCCTGATCCGGAAGATCCGGAAAGCAAGAAAGCTACAAGGAGAGCAGGAA
ACCTAATTATCTGCTTCCTATTCATCTTCTTTCTGTTTGTAACCTTCATTGTTCCAACTCTAAGACAC
TTGCTGTCCTAACACCTGCTATAGGCTATCCACTGCATCATCTCTCCTGCCATACTTCCTACTCACAT
CATATCTATTTTAAAGAAAAAATAGGCCCGAACACTAATCGTGCCGGCAGTGCCACTGCACACACAAC
ACTACACATACAATACACTACAATGGTTGCAGAAGATGCCCCTGTTAGGGCCACTTGCCGAGTATTAT
TTCGAACAACAACTTTAATCTTTCTATGCACACTACTAGCATTAAGCATCTCTATCCTTTATGAGAGT
TTAATAACCCAAAAGCAAATCATGAGCCAAGCAGGCTCAACTGGATCTAATTCTGGATTAGGAAGTAT
CACTGATCTTCTTAATAATATTCTCTCTGTCGCAAATCAGATTATATATAACTCTGCAGTCGCTCTAC
CTCTACAATTGGACACTCTTGAATCAACACTCCTTACAGCCATTAAGTCTCTTCAAACCAGTGACAAG
CTAGAACAGAACTGCTCGTGGAGTGCTGCACTGATTAATGATAATAGATACATTAATGGCATCAATCA
GTTCTATTTTTCAATTGCTGAGGGTCGCAATCTGACACTTGGCCCACTTCTTAATATGCCTAGTTTCA
TTCCAACTGCCACGACACCAGAGGGCTGCACCAGGATCCCATCATTCTCGCTCACTAAGACACACTGG
TGTTATACACACAATGTTATCCTGAATGGATGCCAGGATCATGTATCCTCAAATCAATTTGTTTCtAT
GGGAATCATTGAACCCACTTCTGCCGGGTTTCCATTCTTTCGAACCCTAAAGACTCTATATCTCAGCG
ATGGGGTCAATCGTAAGAGCTGCTCTATCAGTACAGTTCCGGGGGGTTGTATGATGTACTGTTTTGTT
TCTACTCAACCAGAGAGGGATGACTACTTTTCTGCCGCTCCTCCAGAACAACGAATTATTATAATGTA
CTATAATGATACAATCGTGGAGCGCATAATTAATCCACCCGGGGTACTAGATGTATGGGCAACATTGA
ACCCAGGAACAGGAAGCGGGGTATATTATTTAGGTTGGGTGCTCTTTCCAATATATGGCGGCGTGATT
AAAGGTACGAGTTTATGGAATAATCAAGCAAATAAATACTTTATCCCCCAGATGGTTGCTGCTCTCTG
CTCACAAAACCAGGCAACTCAAGTCCAAAATGCTAAGTCATCATACTATAGCAGCTGGTTTGGCAATC
GAATGATTCAGTCTGGGATCCTGGCATGTCCTCTTCGACAGGATCTAACCAATGAGTGTTTAGTTCTG
CCCTTTTCTAATGATCAGGTGCTTATGGGTGCTGAAGGGAGATTATACATGTATGGTGACTCGGTGTA
TTACTATCAAAGAAGCAATAGTTGGTGGCCTATGACCATGCTGTATAAGGTAACCATAACATTCACTA
ATGGTCAGCCATCTGCTATATCAGCTCAGAATGTGCCCACACAGCAGGTCCCTAGACCTGGGACAGGA
GACTGCTCTGCAACCAATAGATGTCCCGGTTTTTGCTTGACAGGAGTGTATGCCGATGCCTGGTTACT
GACCAACCCTTCGTCTACCAGTACATTTGGATCAGAAGCAACCTTCACTGGTTCTTATCTCAACACAG
CAACTCAGCGTATCAATCCGACGATGTATATCGCGAACAACACACAGATCATAAGCTCACAGCAATTT
GGATCAAGCGGTCAAGAAGCAGCATATGGCCACACAACETGTTTTAGGGACACAGGCTCTGTTATGGT
ATACTGTATCTATATTATTGAATTGTCCTCATCTCTCTTAGGACAATTTCAGATTGTCCCATTTATCC
GTCAGGTGACACTATCCTAAAGGCAGAAGCCTTCAGGTCTGACCCAGCCAATCAAAGCATTATACCAG
ACCATGGCCTACCATCGGCTTTAAAGAAAAAAATAGGCCCGGACGGGTTAGCAACAAGCGGCGGCCGC
AATGGTGAGCAAGGGCGAGGAGGATAACATGGCCATCATCAAGGAGTTCATGCGCTTCAAGGTGCACA
TGGAGGGCTCCGTGAACGGCCACGAGTTCGAGATCGAGGGCGAGGGCGAGGGCCGCCCCTACGAGGGC
ACCCAGACCGCCAAGCTGAAGGTGACCAAGGGTGGCCCCCTGCCCTTCGCCTGGGACATCCTGTCCCC
TCAGTTCATGTACGGCTCCAAGGCCTACGTGAAGCACCCCGCCGACATCCCCGACTACTTGAAGCTGT
CCTTCCCCGAGGGCTTCAAGTGGGAGCGCGTGATGAACTTCGAGGACGGCGGCGTGGTGACCGTGACC
CAGGACTCCTCCCTGCAGGACGGCGAGTTCATCTACAAGGTGAAGCTGCGCGGCACCAACTTCCCCTC
CGACGGCCCCGTAATGCAGAAGAAGACCATGGGCTGGGAGGCCTCCTCCGAGCGGATGTACCCCGAGG
ACGGCGCCCTGAAGGGCGAGATCAAGCAGAGGCTGAAGCTGAAGGACGGCGGCCACTACGACGCTGAG
GTCAAGACCACCTACAAGGCCAAGAAGCCCGTGCAGCTGCCCGGCGCCTACAACGTCAACATCAAGTT
GGACATCACCTCCCACAACGAGGACTACACCATCGTGGAACAGTACGAACGCGCCGAGGGCCGCCACT
CCACCGGCGGCATGGACGAGCTGTACAAGTAGGCGGCCGCCTAAGGTCGACTCATGGAATGCATACCA
AACATTATTGACACTAATGACACACAAAATTGGTTTTAAGAAAAACCAAGAGAACAATAGGCCAGAAT
GGCTGGGTCTCGGGAGATATTACTCCCTGAAGTCCATCTCAATTCACCAATTGTAAAGCATAAGCTAT
ACTATTACATTCTACTTGGAAACCTCCCAAATGAGATCGACCTTGACGATTTAGGTCCATTACATAAT
CAAAATTGGAATCAGATAGCACATGAAGAGTCTAACTTAGCTCAACGCTTGGTAAATGTAAGAAATTT
TCTAATTACCCACATCCCTGATCTTAGAAAGGGCCATTGGCAAGAGTATGTCAATGTAATACTGTGGC
CGCGAATTCTTCCCTTGATCCCGGATTTTAAAATCAATGACCAATTGCCTCTGCTCAAAAATTGGGAC
AAGTTAGTTAAAGAATCATGTTCAGTAATCAATGCAGGTACTTCCCAGTGCATTCAGAATCTCAGCTA
TGGACTGACAGGTCGTGGGAACCTCTTTACACGATCACGTGAACTCTCTGGTGACCGCAGGGATATTG
ATCTTAAGACAGTTGTGGCAGCATGGCATGACTCAGACTGGAAAAGAATAAGTGATTTTTGGATTATG
ATCAAATTCCAGATGAGACAATTAATTGTTAGGCAAACAGATCATAATGATTCTGATTTAATCACGTA
TATCGAAAATAGAGAAGGCATAATCATCATAACCCCTGAACTGGTAGCATTATTTAACACTGAGAATC
ATACACTAACATACATGACCTTTGAAATTGTACTGATGGTTTCAGATATGTACGAAGGTCGTCACAAC
ATTTTATCACTATGCACAGTTAGCACTTACCTGAATCCTCTGAAGAAAAGAATAACATATTTATTGAG
CCTTGTAGATAACTTAGCTTTTCAGATAGGTGATGCTGTATATAACATAATTGCTTTGCTAGAATCCT
TTGTATATGCACAGTTGCAAATGTCAGATCCCATCCCAGAACTCAGAGGACAATTCCATGCATTCGTA
TGTTCTGAGATTCTTGATGCACTAAGAGGAACTAATAGTTTCACCCAGGATGAATTAAGAACTGTGAC
AACTAATTTGATATCCCCATTCCAAGATCTGACCCCAGATCTTACGGCTGAATTGCTCTGTATAATGA
GGCTTTGGGGACACCCCATGCTCACTGCCAGTCAAGCTGCAGGAAAGGTACGCGAGTCTATGTGTGCT
GGAAAAGTATTAGACTTTCCCACCATTATGAAAACACTAGCCTTTTTCCATACTATTCTGATCAATGG
ATACAGGAGGAAGCATCATGGAGTATGGCCACCCTTAAACTTACCGGGTAATGCTTCAAAGGGTCTCA
CGGAACTTATGAATGACAATACTGAAATAAGCTATGAATTCACACTTAAGCATTGGAAGGAAGTCTCT
CTTATAAAATTCAAGAAATGTTTTGATGCAGACGCAGGTGAGGAACTCAGTATATTTATGAAAGATAA
GGCAATTAGTGCCCCAAAACAAGACTGGATGAGTGTGTTTAGAAGAAGCCTAATCAAACAGCGCCATC
AGCATCATCAGGTCCCCCTACCAAATCCATTCAATCGACGGCTGTTGCTAAACTTTCTCGGAGATGAC
AAATTCGACCCGAATGTGGAGCTACAGTATGTAACATCAGGTGAGTATCTACATGATGACACGTTTTG
TGCATCATATTCACTAAAAGAGAAGGAAATTAAACCTGATGGTCGAATTTTTGCAAAGTTGACTAAGA
GAATGAGATCATGTCAAGTTATAGCAGAATCTCTTTTAGCGAACCATGCTGGGAAGTTAATGAAAGAG
AATGGTGTTGTGATGAATCAGCTATCATTAACAAAATCACTATTAACAATGAGTCAGATTGGAATAAT
ATCCGAGAAAGCTAGAAAGTCAACTCGAGATAACATAAATCAACCTGGTTTCCAGAATATCCAGAGAA
ATAAATCACATCACTCCAAGCAAGTCAATCAGCGAGATCCAAGTGATGACTTTGAATTGGCAGCATCT
TTTTTAACTACTGATCTCAAAAAATATTGTTTACAATGGAGGTACCAGACAATTATCCCATTTGCTCA
ATCATTAAACAGAATGTATGGTTATCCTCATCTCTTTGAGTGGATTCACTTACGGCTAATGCGTAGTA
CACTTTACGTGGGGGATCCCTTCAACCCACCAGCAGATACCAGTCAATTTGATCTAGATAAAGTAATT
AATGGAGATATCTTCATTGTATCACCCAGAGGTGGAATTGAAGGGCTGTGTCAAAAGGCTTGGACAAT
GATATCTATCGCTGTGATAATTCTATCTGCCACAGAGTCTGGCACACGAGTAATGAGTATGGTGCAGG
GAGATAATCAAGCAATTGCTGTCACCACACGAGTACCAAGGAGCCTGCCGACTCTTGAGAAAAAGACT
ATTGCTTTTAGATCTTGTAATCTATTCTTTGAGAGGTTAAAATGTAATAATTTTGGATTAGGTCACCA
TTTGAAAGAACAAGAGACTATCATTAGTTCTCACTTCTTTGTTTATAGCAAGAGAATATTCTATCAGG
GGAGGATTCTAACGCAAGCCTTAAAAAATGCTAGTAAGCTCTGCTTGACAGCTGATGTCCTAGGAGAA
TGCACCCAATCATCATGTTCTAATCTTGCAACTACTGTCATGAGGTTAACTGAGAATGGTGTTGAAAA
AGATATCTGTTTCTACTTGAATATCTATATGACCATCAAACAGCTCTCCTATGATATCATCTTCCCTC
AAGTGTCAATTCCTGGAGATCAGATCACATTAGAATACATAAATAATCCACACCTGGTATCACGATTG
GCTCTTTTGCCATCCCAGTTAGGAGGTCTAAACTACCTGTCATGCAGTAGGCTGTTCAATCGAAACAT
AGGCGACCCGGTGGTTTCCGCAGTTGCAGATCTTAAGAGATTAATTAAATCAGGATGTATGGATTACT
GGATCCTTTATAACTTATTAGGGAGAAAACCGGGAAACGGCTCATGGGCTACTTTAGCAGCTGACCCG
TACTCAATCAATATAGAGTATCAATACCCTCCAACTACAGCTCTTAAGAGGCACACCCAACAAGCTCT
GATGGAACTCAGTACGAATCCAATGTTACGTGGCATATTCTCTGACAATGCACAGGCAGAAGAAAATA
ACCTTGCTAGGTTTCTCCTGGATAGGGAGGTGATCTTTCCGCGTGTAGCTCACATCATCATTGAGCAA
ACCAGTGTCGGGAGGAGAAAACAGATTCAAGGATATTTGGATTCAACTAGATCGATAATGAGGAAATC
ACTAGAAATTAAGCCCTTATCCAATAGGAAGCTTAATGAAATACTGGATTACAACATCAATTACCTAG
CTTACAATTTGGCATTACTCAAGAATGCTATTGAACCTCCGACTTATTTGAAGGCAATGACACTTGAA
ACATGTAGCATCGACATTGCAAGGAACCTCCGGAAGCTCTCCTGGGCCCCACTCTTGGGTGGGAGAAA
TCTTGAAGGATTAGAGACGCCAGATCCCATTGAAATTACTGCAGGAGCATTAATTGTTGGATCGGGCT
ACTGTGAACAGTGTGCTGCAGGAGACAATCGATTCACATGGTTTTTCTTGCCATCTGGTATCGAGATA
GGAGGGGATCCCCGTGATAATCCTCCTATCCGTGTACCGTACATTGGCTCCAGGACTGATGAGAGGAG
GGTAGCCTCAATGGCATACATCAGGGGTGCCTCGAGTAGCTTAAAAGCAGTTCTTAGACTGGCGGGAG
TGTACATCTGGGCATTCGGAGATACTCTGGAGAATTGGATAGATGCACTGGATTTGTCTCACACTAGA
GTTAACATCACACTTGAACAGCTGCAATCCCTCACCCCACTTCCAACCTCTGCCAATCTAACCCATCG
GTTGGATGATGGCACAACTACCCTAAAGTTTACTCCTGCGAGCTCTTACACCTTTTCAAGTTTCACTC
ATATATCAAATGATGAGCAATACCTGACAATTAATGACAAAACTGCAGATTCAAATATAATCTACCAA
CAGTTAATGATCACTGGACTCGGAATCTTAGAAACATGGAATAATCCCCCAATCAATAGAACATTCGA
AGAATCTACCCTACATTTGCACACTGGTGCATCATGTTGTGTCCGACCTGTGGACTCCTGCATTCTCT
CAGAAGCATTAACAGTCAAGCCACATATTACAGTACCGTACAGCAATAAATTTGTATTTGATGAGGAC
CCGCTATCTGAATATGAAACTGCAAAACTGGAATCGTTATCATTCCAAGCCCAATTAGGCAACATTGA
TGCTGTAGATATGACAGGTAAATTAACATTATTGTCCCAATTCACTGCAAGGCAGATTATCAATGCAA
TCACTGGACTCGATGAGTCTGTCTCTCTTACTAATGATGCCATTGTTGCATCAGACTATGTCTCCAAT
TGGATTAGTGAATGCATGTATACCAAATTAGATGAATTATTTATGTATTGTGGGTGGGAACTACTATT
GGAACTATCCTATCAAATGTATTATCTGAGGGTAGTTGGGTGGAGTAATATAGTGGATTATTCTTACA
TGATCTTGAGAAGAATCCCGGGTGCAGCATTAAACAATCTGGCATCTACATTAAGTCATCCAAAACTT
TTCCGACGAGCTATCAACCTAGATATAGTTGCCCCCTTAAATGCTCCTCATTTTGCATCTCTGGACTA
CATCAAGATGAGTGTGGATGCAATACTCTGGGGCTGTAAAAGAGTCATCAATGTGCTCTCCAATGGAG
GGGACTTAGAATTAGTTGTGACATCTGAAGATAGCCTTATTCTCAGTGACCGATCCATGAATCTCATT
GCAAGGAAATTAACTTTATTATCACTGATTCACCATAATGGTTTGGAACTACCAAAGATTAAGGGGTT
CTCTCCTGATGAGAAGTGTTTCGCTTTGACAGAATTTTTGAGGAAAGTGGTGAACTCAGGGTTGAGTT
CAATAGAGAACCTATCAAATTTTATGTACAATGTGGAGAACCCACGGCTTGCAGCATTCGCCAGCAAC
AATTACTACCTGACCAGAAAATTATTGAATTCAATACGAGATACTGAGTCGGGTCAAGTAGCAGTCAC
CTCATATTATGAATCATTAGAATATATTGATAGTCTTAAGCTAACCCCACATGTGCCTGGCACCTCAT
GCATTGAGGATGATAGTCTATGTACAAATGATTACATAATCTGGATCATAGAGTCTAATGCAAACTTG
GAGAAGTATCCAATTCCAAATAGCCCTGAGGATGATTCCAATTTCCATAACTTTAAGTTGAATGCTCC
ATCGCACCATACCTTACGCCCATTAGGGTTGTCATCAACTGCTTGGTATAAGGGTATAAGCTGCTGCA
GGTACCTTGAGCGATTAAAGCTACCACAAGGTGATCATTTATATATTGqAGAAGGTAGTGGTGCCAGT
ATGACAATCATAGAATACCTATTCCCAGGAAGAAAGATATATTACAATTCTTTATTTAGTAGTGGTGA
CAATCCCCCACAAAGAAATTATGCACCAATGCCTACTCAGTTCATTGAGAGTGTCCCATACAAGCTCT
GGCAAGCACACACAGATCAATATCCCGAGATTTTTGAGGACTTCATCCCTCTATGGAACGGAAACGCC
GCCATGACTGACATAGGAATGACAGCTTGTGTAGAATTCATCATCAATCGAGTCGGCCCAAGGACTTG
CAGTTTAGTACATGTAGATTTGGAATCAAGTGCAAGCTTAAATCAACAATGCCTGTCAAAGCCGATAA
TTAATGCTATCATCACTGCTACAACTGTTTTGTGCCCTCATGGGGTGCTTATTCTGAAATATAGTTGG
TTGCCATTTACTAGATTTAGTACTTTGATCACTTTCTTATGGTGCTACTTTGAGAGAATCACTGTTCT
TAGGAGCACATATTCTGATCCAGCTAATCATGAGGTTTATTTAATTTGTATCCTTGCCAACAACTTTG
CATTCCAGACTGTCTCGCAGGCAACAGGAATGGCGATGACTTTAACTGATCAAGGGTTTACTTTGATA
TCACCTGAAAGAATAAATCAGTATTGGGATGGTCACTTGAAGCAAGAACGTATCGTAGCAGAAGCAAT
TGATAAGGTGGTTCTAGGAGAAAATGCTCTATTTAATTCGAGTGATAATGAATTAATTCTCAAATGTG
GAGGGACACCAAATGCACGGAATCTCATCGATATCGAGCCAGTCGCAACTTTCATAGAATTTGAACAA
TTGATCTGCACAATGTTGACAACCCACTTGAAGGAAATAATTGATATAACAAGGTCTGGAACCCAGGA
TTATGAAAGTTTATTACTCACTCCTTACAATTTAGGTCTTCTTGGTAAAATCAGTACGATAGTGAGAT
TATTAACAGAAAGGATTCTAAATCATACTATCAGGAATTGGTTGATCCTCCCACCTTCGCTCCGGATG
ATCGTGAAGCAGGACTTGGAATTCGGCATATTCAGGATTACTTCCATCCTCAATTCTGATCGGTTCCT
GAAGCTTTCTCCAAATAGGAAATACTTGATTGCACAATTAACTGCAGGCTACATTAGGAAATTGATTG
AGGGGGATTGCAATATCGATCTAACCAGACCTATCCAAAAGCAAATCTGGAAAGCATTAGGTTGTGTA
GTCTATTGTCACGATCCAATGGATCAAAGGGAGTCAACAGAGTTTATTGATATAAATATTAATGAAGA
AATAGACCGCGGGATCGATGGCGAGGAAATCTAAACATATCAAGAATCAGAATTAGTTTAAGAAAAAA
GAAGAGGATTAATCTTGGTTTTCCCCTTGGTGGGTCGGCATGGCATCTCCACCTCCTCGCGGTCCGAC
CTGGGCATCCGAAGGAGGACGCACGTCCACTCGGATGGCTAAGGGAGCGGCCGGGGATCCGGCTGCTA
ACAAAGCCCGAAAGGAAGCTGAGTTGGCTGCTGCCACCGCTGAGCAATAACTAGCATAACCCCTTGGG
GCCTCTAAACGGGTCTTGAGGGGTTTTTTGCTGAAAGGAGGAACTATATCCGGATGGAATCACCAGCT
TGATTTATCTCCAAAATGATTCAAAGAAAACAAATCATATTAAGACTATCCTAAGCACGAACCCATAT
CGTCCTTCAAATCATGGACCCCAAGGGCAGCCTGAGCTGGAGAATCCTGCTGTTCCTGAGCCTGGCCT
TCGAGCTGAGCTACGGCGACGTGCAGGTGGTGGAGAGCGGAGGCGGAGTGGTGCAGCCAGGCGGCAGC
CTGAGACTGAGCTGCGCTGCGAGTGGCTTCACCTTCAGCAGCTTCGGgATGCACTGGGTGAGGCAGGC
ACCCGGCAAGGGCCTGGAGTGGGTGGCCTACATCAACACCGACAGCACCACCATCTACTACGCAGACA
GCGTGAAGGGCAGGTTCACCATCAGCAGGGACAACAGCAAGAACACCCTGTACCTGCAGATGAACTCT
TTGCGGGCAGAAGACACTGCCGTGTACTACTGCGCCAGTGCTGGCCCCTACTATGGCTTTGACTACTG
GGGACAGGGCACCCTGGTGACCGTGAGCAGCGCTAGCACCAAGGGCCtCAGCGTGTTCCCTCTGGCCC
CCAGCAGCAAGAGCACCAGCGGCGGAACCGCCGCCCTGGGCTGCCTGGTGAAGGACTACTTCCCCGAG
CCCGTGACCGTGTCCTGGAACAGCGGCGCTCTGACCAGCGGAGTGCACACCTTCCCTGCCGTGCTGCA
GAGCAGCGGCCTGTACTCCCTGAGCAGCGTGGTGACCGTGCCCAGCAGCAGCCTGGGCACCCAGACCT
ACATCTGCAACGTGAACCACAAGCCCTCCAACACCAAGGTGGACAAGAAGGTGGAGCCTAAGAGCTGC
GACAAGACCCACACCTGCCCTCCCTGCCCCGCCCCCGAGCTGCTGGGCGGACCCAGCGTGTTCCTGTT
CCCTCCCAAGCCCAAGGACACCCTGATGATCAGCCGCACCCCCGAGGTGACCTGCGTGGTGGTGGACG
TGAGCCACGAGGACCCCGAGGTGAAGTTCAACTGGTACGTGGACGGCGTGGAGGTGCACAACGCCAAG
ACCAAGCCTCGGGAGGAGCAGTACAACTCCACCTACCGCGTGGTGAGCGTGCTGACCGTGCTGCACCA
GGACTGGCTGAACGGCAAGGAGTACAAGTGCAAGGTGAGCAACAAGGCCCTGCCCGCTCCCATCGAGA
AGACCATCAGCAAGGCCAAGGGCCAGCCtCGGGAGCCTCAGGTGTACACCCTGCCCCCCAGCCGCGAA
GAGATGACCAAGAACCAGGTGAGCCTGACCTGCCTGGTGAAGGGCTTCTACCCCTCCGACATCGCCGT
GGAGTGGGAGAGCAACGGCCAGCCTGAGAACAACTACAAGACCACCCCTCCCGTGCTGGACAGCGACG
GCAGCTTCTTCCTGTACAGCAAGCTGACCGTGGACAAGTCCCGGTGGCAGCAGGGCAACGTGTTCAGC
TGCAGCGTGATGCACGAGGCCCTGCACAACCACTACACCCAGAAGAGCCTGAGCCTGAGCCCCGGACG
CGCAAAAAGAtctagaGAGGGCAGGGGAAGTCTTCTAACATGCGGGGACGTGGAGGAAAATCCCGGGC
CtATGGAGACCGACACCCTGCTGCTCTGGGTGCTGCTGCTCTGGGTGCCCGGCTCCACCGGAGACATC
GTGATGACCCAGAGTCCACTGAGCCTGCCCGTGACCCCCGGTGAGCCAGCCAGCATCAGCTGCAGGAG
CAGCAAGAGCCTGGTGCACAGCAACGGCATCACCTACCTGTACTGGTACGTGCAGAAGCCCGGACAGA
GCCCCCAGTTGCTCATTTATCAGATGAGCAGCCTGGCAAGCGGCGTGCCCGACAGGTTCAGCGGCTCC
GGCAGCGGCACCGACTTCAGCCTGAAGATCAGCCGGGTGGAGGCCGAGGACGTGGGCGTGTACTACTG
CGGCCAAATTCTGGAGCTGCCCTTCACCTTCGGCCAGGGAACCAAGGTGGAGATCAAGCGGACCGTGG
CCGCCCCCAGCGTGTTCATCTTCCCTCCCAGCGACGAGCAGCTGAAGTCTGGCACCGCCAGCGTGGTG
TGCCTGCTGAACAACTTCTACCCCCGCGAGGCCAAGGTGCAGTGGAAGGTGGACAACGCCCTGCAGAG
CGGCAACAGCCAGGAGAGCGTGACCGAGCAGGACTCCAAGGACAGCACCTACAGCCTGAGCAGCACCC
TGACCCTGAGCAAGGCCGACTACGAGAAGCACAAGGTGTACGCCTGCGAGGTGACCCACCAGGGACTG
TCTAGCCCCGTGACCAAGAGCTTCAACCGGGGCGAGTGCTAATGAGCATTCCACCACTCACGATCTGA
TCTCAGTGAGAAAAATCAACCTGCAACTCTTGGAACAAGATAAGACAGTCATCCATTAGTAATTTTTA
AGAAAAAAACGATAGGACCGAACCT
PIV mini-replicon sequence: pBH276 “minigenome”
CATGTGAGCAAAAGGCCAGCAAAAGGCCAGGAACCGTAAAAAGGCCGCGTTGCTGGCGTTTTTCCATA
GGCTCCGCCCCCCTGACGAGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCCTGTTCCGACCCTGCCGCT
TACCGGATACCTGTCCGCCTTTCTCCCTTCGGGAAGCGTGGCGCTTTCTCATAGCTCACGCTGTAGGT
ATCTCAGTTCGGTGTAGGTCGTTCGCTCCAAGCTGGGCTGTGTGCACGAACCCCCCGTTCAGCCCGAC
CGCTGCGCCTTATCCGGTAACTATCGTCTTGAGTCCAACCCGGTAAGACACGACTTATCGCCACTGGC
AGCAGCCACTGGTAACAGGATTAGCAGAGCGAGGTATGTAGGCGGTGCTACAGAGTTCTTGAAGTGGT
GGCCTAACTACGGCTACACTAGAAGAACAGTATTTGGTATCTGCGCTCTGCTGAAGCCAGTTACCTTC
GGAAAAAGAGTTGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTTCTACGGGGTCTG
ACGCTCAGTGGAACGAAAACTCACGTTAAGGGATTTTGGTCATGAGATTATCAAAAAGGATCTTCACC
TAGATCCTTTTAAATTAAAAATGAAGTTTTAAATCAATCTAAAGTATATATGAGTAAACTTGGTCTGA
CAGTTACCAATGCTTAATCAGTGAGGCACCTATCTCAGCGATCTGTCTATTTCGTTCATCCATAGTTG
CCTGACTCCCCGTCGTGTAGATAACTACGATACGGGAGGGCTTACCATCTGGCCCCAGTGCTGCAATG
ATACCGCGAGACCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGGGAAGCTAGAG
TAAGTAGTTCGCCAGTTAATAGTTTGCGCAACGTTGTTGCCATTGCTACAGGCATCGTGGTGTCACGC
TCGTCGTTTGGTATGGCTTCATTCAGCTCCGGTTCCCAACGATCAAGGCGAGTTACATGATCCCCCAT
GTTGTGCAAAAAAGCGGTTAGCTCCTTCGGTCCTCCGATCGTTGTCAGAAGTAAGTTGGCCGCAGTGT
TATCACTCATGGTTATGGCAGCACTGCATAATTCTCTTACTGTCATGCCATCCGTAAGATGCTTTTCT
GTGACTGGTGAGTACTCAACCAAGTCATTCTGAGAATAGTGTATGCGGCGACCGAGTTGCTCTTGCCC
GGCGTCAATACGGGATAATACCGCGCCACATAGCAGAACTTTAAAAGTGCTCATCATTGGAAAACGTT
CTTCGGGGCGAAAACTCTCAAGGATCTTACCGCTGTTGAGATCCAGTTCGATGTAACCCACTCGTGCA
CCCAACTGATCTTCAGCATCTTTTACTTTCACCAGCGTTTCTGGGTGAGCAAAAACAGGAAGGCAAAA
TGCCGCAAAAAAGGGAATAAGGGCGACACGGAAATGTTGAATACTCATACTCTTCCTTTTTCAATATT
ATTGAAGCATTTATCAGGGTTATTGTCTCATGAGCGGATACATATTTGAATGTATTTAGAAAAATAAA
CAAATAGGGGTTCCGCGCACATTTCCCCGAAAAGTGCCACCTGACGTCTAAGAAACCATTATTATCAT
GACATTAACCTATAAAAATAGGCGTATCACGAGGCCCTTTCGTCTCGCGCGTTTCGGTGATGACGGTG
AAAACCTCTGACACATGCAGCTCCCGGAGACGGTCACAGCTTGTCTGTAAGCGGATGCCGGGAGCAGA
CAAGCCCGTCAGGGCGCGTCAGCGGGTGTTGGCGGGTGTCGGGGCTGGCTTAACTATGCGGCATCAGA
GCAGATTGTACTGAGAGTGCACCATATGCGGTGTGAAATACCGCACAGATGCGTAAGGAGAAAATACC
GCATCAGGCTAATACGACTCACTATAGGGACCAAGGGGAAAATGAAGTGGTGACTCAAATCATCGAAG
ACCCTCGAGATTACATAGGTCCGGAACCTATGGCCTTCGTGACCGACCTCGAGTCAGAGTAGTTCAAT
AAGGACCTATCAAGTTTGGGCAATTTTTCGTCCCCGACACAAAAATGTCATCCGTGCTTAAAGCATAT
GAGCGATTCACGCTCACTCAAGAACTGCAAGATCAGAGTGAGGAAGGTACAATCCCACCTACAACACT
AAAACCGGTAATCAGGGTATTTATACTAACCTCTAATAACCCAGAGCTAAGATCCCGGCTTCTTCTAT
TCTGCCTACGGATTGTTCTCAGTAATGGTGCAAGGGATTCCCATCGCTTTGGAGCATTACTCACAATG
TTTTCGCTACCATCAGCCACAATGCTCAATCATGTCAAATTAGCTGACCAGTCACCAGAAGCTGATAT
CGAAAGGGTAGAGATCGATGGCTTTGAGGAGGGATCATTCCGCTTAATCCCCAATGCtCGTTCAGGTA
TGAGCCGTGGAGAGATCAATGCCTATGCTGCACTTGCAGAAGATCTACCTGACACACTAAACCATGCA
ACACCTTTCGTTGATTCCGAAGTCGAGGGAACTGCATGGGATGAGATTGAGACTTTCTTAGATATGTG
TTACAGTGTCCTAATGCAGGCATGGATAGTGACTTGCAAGTGCATGACTGCGCCAGACCAACCTGCTG
CTTCTATTGAGAAACGCCTGCAAAAATATCGTCAGCAAGGCAGGATCAACCCGAGATATCTCCTGCAA
CCGGAGGCTCGACGAATAATCCAGAATGTAATCCGGAAGGGAATGGTGGTCAGACATTTCCTCACCTT
TGAACTGCAGCTTGCCCGAGCACAAAGCCTTGTATCAAATAGGTATTATGCTATGGTAGGGGATGTTG
GAAAGTATATAGAGAATTGTGGAATGGGAGGCTTCTTTTTGACACTAAAATATGCATTAGGAACTAGA
TGGCCCACACTTGCTTTAGCTGCATTTTCAGGAGAGCTAACAAAGCTAAAGTCCCTCATGGCATTATA
CCAGACCCTTGGTGAGCAGGCCCGATATTTGGCCCTATTGGAGTCACCACATTTGATGGATTTTGCTG
CAGCAAACTACCCACTGCTATATAGCTATGCTATGGGAATAGGCTATGTGTTAGATGTCAACATGAGG
AACTACGCTTTCTCCAGATCATACATGAACAAGACATATTTCCAATTGGGAATGGAAACTGCAAGAAA
ACAACAGGGTGCAGTTGACATGAGGATGGCAGAAGATCTCGGTCTAACTCAAGCCGAACGCACCGAGA
TGGCAAATACACTTGCCAAATTGACCACAGCAAATCGAGGGGCAGACACCAGGGGAGGAGTCAACCCG
TTCTCATCTGTCACTGGGACAACTCAGGTGCCCGCTGCAGCAACAGGTGACACACTCGAGAGTTACAT
GGCAGCGGATCGACTGAGGCAGAGATATGCTGATGCAGGCACCCATGATGATGAGATGCCACCATTGG
AAGAGGAGGAAGAGGACGACACATCTGCAGGTCCACGCACTGGACCAACTCTTGAACAAGTGGCCTTG
GACATCCAGAACGCAGCAGTTGGAGCTCCCATCCATACAGATGACCTGAATGCCGCACTGGGTGATCT
TGACATCTAGACAATTCAGATCCCAATCTAAAATTGACATACCTAATTGATTAGTTAGATGGAACTAC
AGTGGATTCCATAAGGTTCCTGCCTACCATCGGCTTTAAAGAAAAAAATAGGCCCGGACGGGTTAGCA
ACAAGCGACTGCCGGTGCCAACAGCGCAATCCACAATCTACAATGGATCCCACTGATCTGAGCTTCTC
CCCAGATGAGATCAATAAGCTCATAGAGACAGGCCTGAATACTGTAGAGTATTTTACTTCCCAACAAG
TCACAGGAACATCCTCTCTTGGAAAGAATACAATACCACCAGGGGTCACAGGACTACTAACCAATGCT
GCAGAGGCAAAGATCCAAGAGTCAACTAACCATCAGAAGGGCTCAGTTGGTGGGGGTGCAAAACCAAA
GAAACCGCGACCAAAAATTGCCATTGTGCCAGCAGATGACAAAACAGTGCCCGGAAAGCCGATCCCAA
ACCCTCTATTAGGTCTGGACTCCACCCCGAGCACCCAAACTGTGCTTGATCTAAGTGGGAAAACATTA
CCATCAGGATCCTATAAGGGGGTTAAGCTTGCGAAATTTGGAAAAGAAAATCTGATGACACGGTTCAT
CGAGGAACCCAGAGAGAATCCTATCGCAACCAGTTCCCCCATCGATTTTAAGAGGGGCAGGGATACCG
GCGGGTTCCATAGAAGGGAGTACTCAATCGGATGGGTGGGAGATGAAGTCAAGGTCACTGAGTGGTGC
AATCCATCCTGTTCTCCAATCACCGCTGCAGCAAGGCGATTTGAATGCACTTGTCACCAGTGTCCAGT
CACTTGCTCTGAATGTGAACGAGATACTTAATACAGTGAGAAATTTGGACTCTCGGATGAATCAACTG
GAGACAAAAGTAGATCGCATTCTCTCATCTCAGTCTCTAATCCAGACCATCAAGAATGACATAGTTGG
ACTTAAAGCAGGGATGGCTACTTTAGAAGGAATGATTACAACTGTGAAAATCATGGACCCGGGAGTTC
CCAGTAATGTTACTGTGGAAGATGTACGCAAGACACTAAGTAACCATGCTGTTGTTGTGCCAGAATCA
TTCAATGATAGTTTCTTGACTCAATCTGAAGATGTAATTTCACTTGATGAGTTGGCTCGACCAACTGC
AACAAGTGTTAAGAAGATTGTCAGGAAGGTTCCTCCTCAGAAGGATCTGACTGGATTGAAGATTACAC
TAGAGCAATTGGCAAAGGATTGCATCAGCAAACCGAAGATGAGGGAAGAGTATCTCCTCAAAATCAAC
CAGGCTTCCAGTGAGGCTCAGCTAATTGACCTCAAGAAAGCAATCATCCGCAGTGCAATTTGATCAAG
AAACACCCAATTACACTACACTGGTATGACACTGTACTAACCCTGAGGGTTTTAGAAAAAACgcCAAG
AGAACAATAGGCCAGAATGGCTGGGTCTCGGGAGATATTACTCCCTGAAGTCCATCTCAATTCACCAA
TTGTAAAGCATAAGCTATACTATTACATTCTACTTGGAAACCTCCCAAATGAGATCGACCTTGACGAT
TTAGGTCCATTACATAATCAAAATTGGAATCAGATAGCACATGAAGAGTCTAACTTAGCTCAACGCTT
GGTAAATGTAAGAAATTTTCTAATTACCCACATCCCTGATCTTAGAAAGGGCCATTGGCAAGAGTATG
TCAATGTAATACTGTGGCCGCGAATTCTTCCCTTGATCCCGGATTTTAAAATCAATGACCAATTGCCT
CTGCTCAAAAATTGGGACAAGTTAGTTAAAGAATCATGTTCAGTAATCAATGCAGGTACTTCCCAGTG
CATTCAGAATCTCAGCTATGGACTGACAGGTCGTGGGAACCTCTTTACACGATCACGTGAACTCTCTG
GTGACCGCAGGGATATTGATCTTAAGACAGTTGTGGCAGCATGGCATGACTCAGACTGGAAAAGAATA
AGTGATTTTTGGATTATGATCAAATTCCAGATGAGACAATTAATTGTTAGGCAAACAGATCATAATGA
TTCTGATTTAATCACGTATATCGAAAATAGAGAAGGCATAATCATCATAACCCCTGAACTGGTAGCAT
TATTTAACACTGAGAATCATACACTAACATACATGACCTTTGAAATTGTACTGATGGTTTCAGATATG
TACGAAGGTCGTCACAACATTTTATCACTATGCACAGTTAGCACTTACCTGAATCCTCTGAAGAAAAG
AATAACATATTTATTGAGCCTTGTAGATAACTTAGCTTTTCAGATAGGTGATGCTGTATATAACATAA
TTGCTTTGCTAGAATCCTTTGTATATGCACAGTTGCAAATGTCAGATCCCATCCCAGAACTCAGAGGA
CAATTCCATGCATTCGTATGTTCTGAGATTCTTGATGCACTAAGAGGAACTAATAGTTTCACCCAGGA
TGAATTAAGAACTGTGACAACTAATTTGATATCCCCATTCCAAGATCTGACCCCAGATCTTACGGCTG
AATTGCTCTGTATAATGAGGCTTTGGGGACACCCCATGCTCACTGCCAGTCAAGCTGCAGGAAAGGTA
CGCGAGTCTATGTGTGCTGGAAAAGTATTAGACTTTCCCACCATTATGAAAACACTAGCCTTTTTCCA
TACTATTCTGATCAATGGATACAGGAGGAAGCATCATGGAGTATGGCCACCCTTAAACTTACCGGGTA
ATGCTTCAAAGGGTCTCACGGAACTTATGAATGACAATACTGAAATAAGCTATGAATTCACACTTAAG
CATTGGAAGGAAGTCTCTCTTATAAAATTCAAGAAATGTTTTGATGCAGACGCAGGTGAGGAACTCAG
TATATTTATGAAAGATAAGGCAATTAGTGCCCCAAAACAAGACTGGATGAGTGTGTTTAGAAGAAGCC
TAATCAAACAGCGCCATCAGCATCATCAGGTCCCCCTACCAAATCCATTCAATCGACGGCTGTTGCTA
AACTTTCTCGGAGATGACAAATTCGACCCGAATGTGGAGCTACAGTATGTAACATCAGGTGAGTATCT
ACATGATGACACGTTTTGTGCATCATATTCACTAAAAGAGAAGGAAATTAAACCTGATGGTCGAATTT
TTGCAAAGTTGACTAAGAGAATGAGATCATGTCAAGTTATAGCAGAATCTCTTTTAGCGAACCATGCT
GGGAAGTTAATGAAAGAGAATGGTGTTGTGATGAATCAGCTATCATTAACAAAATCACTATTAACAAT
GAGTCAGATTGGAATAATATCCGAGAAAGCTAGAAAGTCAACTCGAGATAACATAAATCAACCTGGTT
TCCAGAATATCCAGAGAAATAAATCACATCACTCCAAGCAAGTCAATCAGCGAGATCCAAGTGATGAC
TTTGAATTGGCAGCATCTTTTTTAACTACTGATCTCAAAAAATATTGTTTACAATGGAGGTACCAGAC
AATTATCCCATTTGCTCAATCATTAAACAGAATGTATGGTTATCCTCATCTCTTTGAGTGGATTCACT
TACGGCTAATGCGTAGTACACTTTACGTGGGGGATCCCTTCAACCCACCAGCAGATACCAGTCAATTT
GATCTAGATAAAGTAATTAATGGAGATATCTTCATTGTATCACCCAGAGGTGGAATTGAAGGGCTGTG
TCAAAAGGCTTGGACAATGATATCTATCGCTGTGATAATTCTATCTGCCACAGAGTCTGGCACACGAG
TAATGAGTATGGTGCAGGGAGATAATCAAGCAATTGCTGTCACCACACGAGTACCAAGGAGCCTGCCG
ACTCTTGAGAAAAAGACTATTGCTTTTAGATCTTGTAATCTATTCTTTGAGAGGTTAAAATGTAATAA
TTTTGGATTAGGTCACCATTTGAAAGAACAAGAGACTATCATTAGTTCTCACTTCTTTGTTTATAGCA
AGAGAATATTCTATCAGGGGAGGATTCTAACGCAAGCCTTAAAAAATGCTAGTAAGCTCTGCTTGACA
GCTGATGTCCTAGGAGAATGCACCCAATCATCATGTTCTAATCTTGCAACTACTGTCATGAGGTTAAC
TGAGAATGGTGTTGAAAAAGATATCTGTTTCTACTTGAATATCTATATGACCATCAAACAGCTCTCCT
ATGATATCATCTTCCCTCAAGTGTCAATTCCTGGAGATCAGATCACATTAGAATACATAAATAATCCA
CACCTGGTATCACGATTGGCTCTTTTGCCATCCCAGTTAGGAGGTCTAAACTACCTGTCATGCAGTAG
GCTGTTCAATCGAAACATAGGCGACCCGGTGGTTTCCGCAGTTGCAGATCTTAAGAGATTAATTAAAT
CAGGATGTATGGATTACTGGATCCTTTATAACTTATTAGGGAGAAAACCGGGAAACGGCTCATGGGCT
ACTTTAGCAGCTGACCCGTACTCAATCAATATAGAGTATCAATACCCTCCAACTACAGCTCTTAAGAG
GCACACCCAACAAGCTCTGATGGAACTCAGTACGAATCCAATGTTACGTGGCATATTCTCTGACAATG
CACAGGCAGAAGAAAATAACCTTGCTAGGTTTCTCCTGGATAGGGAGGTGATCTTTCCGCGTGTAGCT
CACATCATCATTGAGCAAACCAGTGTCGGGAGGAGAAAACAGATTCAAGGATATTTGGATTCAACTAG
ATCGATAATGAGGAAATCACTAGAAATTAAGCCCTTATCCAATAGGAAGCTTAATGAAATACTGGATT
ACAACATCAATTACCTAGCTTACAATTTGGCATTACTCAAGAATGCTATTGAACCTCCGACTTATTTG
AAGGCAATGACACTTGAAACATGTAGCATCGACATTGCAAGGAACCTCCGGAAGCTCTCCTGGGCCCC
ACTCTTGGGTGGGAGAAATCTTGAAGGATTAGAGACGCCAGATCCCATTGAAATTACTGCAGGAGCAT
TAATTGTTGGATCGGGCTACTGTGAACAGTGTGCTGCAGGAGACAATCGATTCACATGGTTTTTCTTG
CCATCTGGTATCGAGATAGGAGGGGATCCCCGTGATAATCCTCCTATCCGTGTACCGTACATTGGCTC
CAGGACTGATGAGAGGAGGGTAGCCTCAATGGCATACATCAGGGGTGCCTCGAGTAGCTTAAAAGCAG
TTCTTAGACTGGCGGGAGTGTACATCTGGGCATTCGGAGATACTCTGGAGAATTGGATAGATGCACTG
GATTTGTCTCACACTAGAGTTAACATCACACTTGAACAGCTGCAATCCCTCACCCCACTTCCAACCTC
TGCCAATCTAACCCATCGGTTGGATGATGGCACAACTACCCTAAAGTTTACTCCTGCGAGCTCTTACA
CCTTTTCAAGTTTCACTCATATATCAAATGATGAGCAATACCTGACAATTAATGACAAAACTGCAGAT
TCAAATATAATCTACCAACAGTTAATGATCACTGGACTCGGAATCTTAGAAACATGGAATAATCCCCC
AATCAATAGAACATTCGAAGAATCTACCCTACATTTGCACACTGGTGCATCATGTTGTGTCCGACCTG
TGGACTCCTGCATTCTCTCAGAAGCATTAACAGTCAAGCCACATATTACAGTACCGTACAGCAATAAA
TTTGTATTTGATGAGGACCCGCTATCTGAATATGAAACTGCAAAACTGGAATCGTTATCATTCCAAGC
CCAATTAGGCAACATTGATGCTGTAGATATGACAGGTAAATTAACATTATTGTCCCAATTCACTGCAA
GGCAGATTATCAATGCAATCACTGGACTCGATGAGTCTGTCTCTCTTACTAATGATGCCATTGTTGCA
TCAGACTATGTCTCCAATTGGATTAGTGAATGCATGTATACCAAATTAGATGAATTATTTATGTATTG
TGGGTGGGAACTACTATTGGAACTATCCTATCAAATGTATTATCTGAGGGTAGTTGGGTGGAGTAATA
TAGTGGATTATTCTTACATGATCTTGAGAAGAATCCCGGGTGCAGCATTAAACAATCTGGCATCTACA
TTAAGTCATCCAAAACTTTTCCGACGAGCTATCAACCTAGATATAGTTGCCCCCTTAAATGCTCCTCA
TTTTGCATCTCTGGACTACATCAAGATGAGTGTGGATGCAATACTCTGGGGCTGTAAAAGAGTCATCA
ATGTGCTCTCCAATGGAGGGGACTTAGAATTAGTTGTGACATCTGAAGATAGCCTTATTCTCAGTGAC
CGATCCATGAATCTCATTGCAAGGAAATTAACTTTATTATCACTGATTCACCATAATGGTTTGGAACT
ACCAAAGATTAAGGGGTTCTCTCCTGATGAGAAGTGTTTCGCTTTGACAGAATTTTTGAGGAAAGTGG
TGAACTCAGGGTTGAGTTCAATAGAGAACCTATCAAATTTTATGTACAATGTGGAGAACCCACGGCTT
GCAGCATTCGCCAGCAACAATTACTACCTGACCAGAAAATTATTGAATTCAATACGAGATACTGAGTC
GGGTCAAGTAGCAGTCACCTCATATTATGAATCATTAGAATATATTGATAGTCTTAAGCTAACCCCAC
ATGTGCCTGGCACCTCATGCATTGAGGATGATAGTCTATGTACAAATGATTACATAATCTGGATCATA
GAGTCTAATGCAAACTTGGAGAAGTATCCAATTCCAAATAGCCCTGAGGATGATTCCAATTTCCATAA
CTTTAAGTTGAATGCTCCATCGCACCATACCTTACGCCCATTAGGGTTGTCATCAACTGCTTGGTATA
AGGGTATAAGCTGCTGCAGGTACCTTGAGCGATTAAAGCTACCACAAGGTGATCATTTATATATTGgA
GAAGGTAGTGGTGCCAGTATGACAATCATAGAATACCTATTCCCAGGAAGAAAGATATATTACAATTC
TTTATTTAGTAGTGGTGACAATCCCCCACAAAGAAATTATGCACCAATGCCTACTCAGTTCATTGAGA
GTGTCCCATACAAGCTCTGGCAAGCACACACAGATCAATATCCCGAGATTTTTGAGGACTTCATCCCT
CTATGGAACGGAAACGCCGCCATGACTGACATAGGAATGACAGCTTGTGTAGAATTCATCATCAATCG
AGTCGGCCCAAGGACTTGCAGTTTAGTACATGTAGATTTGGAATCAAGTGCAAGCTTAAATCAACAAT
GCCTGTCAAAGCCGATAATTAATGCTATCATCACTGCTACAACTGTTTTGTGCCCTCATGGGGTGCTT
ATTCTGAAATATAGTTGGTTGCCATTTACTAGATTTAGTACTTTGATCACTTTCTTATGGTGCTACTT
TGAGAGAATCACTGTTCTTAGGAGCACATATTCTGATCCAGCTAATCATGAGGTTTATTTAATTTGTA
TCCTTGCCAACAACTTTGCATTCCAGACTGTCTCGCAGGCAACAGGAATGGCGATGACTTTAACTGAT
CAAGGGTTTACTTTGATATCACCTGAAAGAATAAATCAGTATTGGGATGGTCACTTGAAGCAAGAACG
TATCGTAGCAGAAGCAATTGATAAGGTGGTTCTAGGAGAAAATGCTCTATTTAATTCGAGTGATAATG
AATTAATTCTCAAATGTGGAGGGACACCAAATGCACGGAATCTCATCGATATCGAGCCAGTCGCAACT
TTCATAGAATTTGAACAATTGATCTGCACAATGTTGACAACCCACTTGAAGGAAATAATTGATATAAC
AAGGTCTGGAACCCAGGATTATGAAAGTTTATTACTCACTCCTTACAATTTAGGTCTTCTTGGTAAAA
TCAGTACGATAGTGAGATTATTAACAGAAAGGATTCTAAATCATACTATCAGGAATTGGTTGATCCTC
CCACCTTCGCTCCGGATGATCGTGAAGCAGGACTTGGAATTCGGCATATTCAGGATTACTTCCATCCT
CAATTCTGATCGGTTCCTGAAGCTTTCTCCAAATAGGAAATACTTGATTGCACAATTAACTGCAGGCT
ACATTAGGAAATTGATTGAGGGGGATTGCAATATCGATCTAACCAGACCTATCCAAAAGCAAATCTGG
AAAGCATTAGGTTGTGTAGTCTATTGTCACGATCCAATGGATCAAAGGGAGTCAACAGAGTTTATTGA
TATAAATATTAATGAAGAAATAGACCGCGGGATCGATGGCGAGGAAATCTAAACATATCAAGAATCAG
AATTAGTTTAAGAAAAAAGAAGAGGATTAATCTTGGTTTTCCCCTTGGTGGGTCGGCATGGCATCTCC
ACCTCCTCGCGGTCCGACCTGGGCATCCGAAGGAGGACGCACGTCCACTCGGATGGCTAAGGGAGCGG
CCGGGGATCCGGCTGCTAACAAAGCCCGAAAGGAAGCTGAGTTGGCTGCTGCCACCGCTGAGCAATAA
CTAGCATAACCCCTTGGGGCCTCTAAACGGGTCTTGAGGGGTTTTTTGCTGAAAGGAGGAACTATATC
CGGAT
Indel IRES-mCh-2A-Puro insert in T7 promoter orientation
CATGTGAGCAAAAGGCCAGCAAAAGGCCAGGAACCGTAAAAAGGCCGCGTTGCTGGCGTTTTTCCATA
GGCTCCGCCCCCCTGACGAGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCCTGTTCCGACCCTGCCGCT
TACCGGATACCTGTCCGCCTTTCTCCCTTCGGGAAGCGTGGCGCTTTCTCATAGCTCACGCTGTAGGT
ATCTCAGTTCGGTGTAGGTCGTTCGCTCCAAGCTGGGCTGTGTGCACGAACCCCCCGTTCAGCCCGAC
CGCTGCGCCTTATCCGGTAACTATCGTCTTGAGTCCAACCCGGTAAGACACGACTTATCGCCACTGGC
AGCAGCCACTGGTAACAGGATTAGCAGAGCGAGGTATGTAGGCGGTGCTACAGAGTTCTTGAAGTGGT
GGCCTAACTACGGCTACACTAGAAGAACAGTATTTGGTATCTGCGCTCTGCTGAAGCCAGTTACCTTC
GGAAAAAGAGTTGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTTCTACGGGGTCTG
ACGCTCAGTGGAACGAAAACTCACGTTAAGGGATTTTGGTCATGAGATTATCAAAAAGGATCTTCACC
TAGATCCTTTTAAATTAAAAATGAAGTTTTAAATCAATCTAAAGTATATATGAGTAAACTTGGTCTGA
CAGTTACCAATGCTTAATCAGTGAGGCACCTATCTCAGCGATCTGTCTATTTCGTTCATCCATAGTTG
CCTGACTCCCCGTCGTGTAGATAACTACGATACGGGAGGGCTTACCATCTGGCCCCAGTGCTGCAATG
ATACCGCGAGACCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGGGAAGCTAGAG
TAAGTAGTTCGCCAGTTAATAGTTTGCGCAACGTTGTTGCCATTGCTACAGGCATCGTGGTGTCACGC
TCGTCGTTTGGTATGGCTTCATTCAGCTCCGGTTCCCAACGATCAAGGCGAGTTACATGATCCCCCAT
GTTGTGCAAAAAAGCGGTTAGCTCCTTCGGTCCTCCGATCGTTGTCAGAAGTAAGTTGGCCGCAGTGT
TATCACTCATGGTTATGGCAGCACTGCATAATTCTCTTACTGTCATGCCATCCGTAAGATGCTTTTCT
GTGACTGGTGAGTACTCAACCAAGTCATTCTGAGAATAGTGTATGCGGCGACCGAGTTGCTCTTGCCC
GGCGTCAATACGGGATAATACCGCGCCACATAGCAGAACTTTAAAAGTGCTCATCATTGGAAAACGTT
CTTCGGGGCGAAAACTCTCAAGGATCTTACCGCTGTTGAGATCCAGTTCGATGTAACCCACTCGTGCA
CCCAACTGATCTTCAGCATCTTTTACTTTCACCAGCGTTTCTGGGTGAGCAAAAACAGGAAGGCAAAA
TGCCGCAAAAAAGGGAATAAGGGCGACACGGAAATGTTGAATACTCATACTCTTCCTTTTTCAATATT
ATTGAAGCATTTATCAGGGTTATTGTCTCATGAGCGGATACATATTTGAATGTATTTAGAAAAATAAA
CAAATAGGGGTTCCGCGCACATTTCCCCGAAAAGTGCCACCTGACGTCTAAGAAACCATTATTATCAT
GACATTAACCTATAAAAATAGGCGTATCACGAGGCCCTTTCGTCTCGCGCGTTTCGGTGATGACGGTG
AAAACCTCTGACACATGCAGCTCCCGGAGACGGTCACAGCTTGTCTGTAAGCGGATGCCGGGAGCAGA
CAAGCCCGTCAGGGCGCGTCAGCGGGTGTTGGCGGGTGTCGGGGCTGGCTTAACTATGCGGCATCAGA
GCAGATTGTACTGAGAGTGCACCATATGCGGTGTGAAATACCGCACAGATGCGTAAGGAGAAAATACC
GCATCAGGCTAATACGACTCACTATAGGGACCAAGGGGAAAACCAAGATTAATCCTCTTCTTTTTTCT
TAAACTAATTCTGATTCTTGATATGTTTAGATTTCCTCGCCATCGATCCCGCGGTCTATTTCTgcTCA
GGCACCGGGCTTGCGGGTCATGCACCAGGTGCGCGGTCCTTCGGGCACCTCGACGTCGGCGGTGACGG
TGAAGCCGAGCCGCTCGTAGAAGGGGAGGTTGCGGGGCGCGGAGGTCTCCAGGAAGGCGGGCACCCCG
GCGCGCTCGGCCGCCTCCACTCCGGGGAGCACGACGGCGCTGCCCAGACCCTTGCCCTGGTGGTCGGG
CGAGACTCCGACGGTGGCCAGGAACCACGCGGGCTCCTTGGGCCGGTGCGGCGCCAGGAGGCCTTCCA
TCTGTTGCTGCGCGGCCAGCCGGGAACCGCTCAACTCGGCCATGCGCGGGCCGATCTCGGCGAACACC
GCCCCCGCTTCGACGCTCTCCGGCGTGGTCCAGACCGCCACCGCGGCGCCGTCGTCCGCGACCCACAC
CTTGCCGATGTCGAGCCCGACGCGCGTGAGGAAGAGTTCTTGCAGCTCGGTGACCCGCTCGATGTGGC
GGTCCGGATCGACGGTGTGGCGCGTGGCGGGGTAGTCGGCGAACGCGGCGGCGAGGGTGCGTACGGCC
CTGGGGACGTCGTCGCGGGTGGCGAGGCGCACCGTGGGCTTGTACTCGGTCATTGGGCCAGGATTCTC
CTCGACGTCACCGCATGTTAGCAGACTTCCTCTGCCCTCTCCCTTGTACAGCTCGTCCATGCCGCCGG
TGGAGTGGCGGCCCTCGGCGCGTTCGTACTGTTCCACGATGGTGTAGTCCTCGTTGTGGGAGGTGATG
TCCAACTTGATGTTGACGTTGTAGGCGCCGGGCAGCTGCACGGGCTTCTTGGCCTTGTAGGTGGTCTT
GACCTCAGCGTCGTAGTGGCCGCCGTCCTTCAGCTTCAGCCTCTGCTTGATCTCGCCCTTCAGGGCGC
CGTCCTCGGGGTACATCCGCTCGGAGGAGGCCTCCCAGCCCATGGTCTTCTTCTGCATTACGGGGCCG
TCGGAGGGGAAGTTGGTGCCGCGCAGCTTCACCTTGTAGATGAACTCGCCGTCCTGCAGGGAGGAGTC
CTGGGTCACGGTCACCACGCCGCCGTCCTCGAAGTTCATCACGCGCTCCCACTTGAAGCCCTCGGGGA
AGGACAGCTTCAAGTAGTCGGGGATGTCGGCGGGGTGCTTCACGTAGGCCTTGGAGCCGTACATGAAC
TGAGGGGACAGGATGTCCCAGGCGAAGGGCAGGGGGCCACCCTTGGTCACCTTCAGCTTGGCGGTCTG
GGTGCCCTCGTAGGGGCGGCCCTCGCCCTCGCCCTCGATCTCGAACTCGTGGCCGTTCACGGAGCCCT
CCATGTGCACCTTGAAGCGCATGAACTCCTTGATGATGGCCATGTTATCCTCCTCGCCCTTGCTCACC
ATcacacAATTCGCTTTATGATAACAATCTGTGATTGTCACCATAAGCAGCCACAATAAAATAAAAGG
AAACACGGACACCCAAAGTAGTCGGTTCCGCCACGGACTTGCGCGTTACGACAGGCCAATCACTGGTT
TGTGACCACCTGCTCCGAGGTTGGGATTAGCCGCATTCAGGGGCCGGAGGATTCTTATGTAGCTCAAT
AGGCTCTTCACACCTTGTTCACAACTAGCGTCCCATGGCGTTAGCCATAGGTAGGCCGCCAACGCAGC
CTGGACCACCGTCACCGGTGAGGGATGTCCAGACTCATCAGCCTAAGCTACACTCTGGGGTTGAGTGC
TGAGCGCAACGCATCGAAGATTCCGAGGTGGTACTGGGCTTCTCGAAGTACATAAGCGGATAACGGAT
CCGTCGCTTTCAACCACGCAAGCAGTCTATACGACATCACCGGGGAAACAGAAGTGCTTGTTCGTGGT
GGTACTGGTTTGTACCCCCTTCTATTGAACTTGGTTTTGTGCGTCTAAGTTACGGGAAGGGAGTATAA
AACAGGCGTACAAGGGTACCGCAATACCGGAGTACTAGCCGCCACGTGGGCCTCTGGGGTGGGTACAA
CCCCAGAGCTGTTTTAAGCTAACTACTCTGACTCGAGGTCGGTCACGAAGGCCATAGGTTCCGGACCT
ATGTAATCTCGAGGGTCTTCGATGATTTGAGTCACCACTTCATTTTCCCCTTGGTGGGTCGGCATGGC
ATCTCCACCTCCTCGCGGTCCGACCTGGGCATCCGAAGGAGGACGCACGTCCACTCGGATGGCTAAGG
GAGCGGCCGGGGATCCGGCTGCTAACAAAGCCCGAAAGGAAGCTGAGTTGGCTGCTGCCACCGCTGAG
CAATAACTAGCATAACCCCTTGGGGCCTCTAAACGGGTCTTGAGGGGTTTTTTGCTGAAAGGAGGAAC
TATATCCGGAT
pIndel-IRES-PIV5-F-2A-Puro
CATGTGAGCAAAAGGCCAGCAAAAGGCCAGGAACCGTAAAAAGGCCGCGTTGCTGGCGTTTTTCCATA
GGCTCCGCCCCCCTGACGAGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCCTGTTCCGACCCTGCCGCT
TACCGGATACCTGTCCGCCTTTCTCCCTTCGGGAAGCGTGGCGCTTTCTCATAGCTCACGCTGTAGGT
ATCTCAGTTCGGTGTAGGTCGTTCGCTCCAAGCTGGGCTGTGTGCACGAACCCCCCGTTCAGCCCGAC
CGCTGCGCCTTATCCGGTAACTATCGTCTTGAGTCCAACCCGGTAAGACACGACTTATCGCCACTGGC
AGCAGCCACTGGTAACAGGATTAGCAGAGCGAGGTATGTAGGCGGTGCTACAGAGTTCTTGAAGTGGT
GGCCTAACTACGGCTACACTAGAAGAACAGTATTTGGTATCTGCGCTCTGCTGAAGCCAGTTACCTTC
GGAAAAAGAGTTGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTTCTACGGGGTCTG
ACGCTCAGTGGAACGAAAACTCACGTTAAGGGATTTTGGTCATGAGATTATCAAAAAGGATCTTCACC
TAGATCCTTTTAAATTAAAAATGAAGTTTTAAATCAATCTAAAGTATATATGAGTAAACTTGGTCTGA
CAGTTACCAATGCTTAATCAGTGAGGCACCTATCTCAGCGATCTGTCTATTTCGTTCATCCATAGTTG
CCTGACTCCCCGTCGTGTAGATAACTACGATACGGGAGGGCTTACCATCTGGCCCCAGTGCTGCAATG
ATACCGCGAGACCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGGGAAGCTAGAG
TAAGTAGTTCGCCAGTTAATAGTTTGCGCAACGTTGTTGCCATTGCTACAGGCATCGTGGTGTCACGC
TCGTCGTTTGGTATGGCTTCATTCAGCTCCGGTTCCCAACGATCAAGGCGAGTTACATGATCCCCCAT
GTTGTGCAAAAAAGCGGTTAGCTCCTTCGGTCCTCCGATCGTTGTCAGAAGTAAGTTGGCCGCAGTGT
TATCACTCATGGTTATGGCAGCACTGCATAATTCTCTTACTGTCATGCCATCCGTAAGATGCTTTTCT
GTGACTGGTGAGTACTCAACCAAGTCATTCTGAGAATAGTGTATGCGGCGACCGAGTTGCTCTTGCCC
GGCGTCAATACGGGATAATACCGCGCCACATAGCAGAACTTTAAAAGTGCTCATCATTGGAAAACGTT
CTTCGGGGCGAAAACTCTCAAGGATCTTACCGCTGTTGAGATCCAGTTCGATGTAACCCACTCGTGCA
CCCAACTGATCTTCAGCATCTTTTACTTTCACCAGCGTTTCTGGGTGAGCAAAAACAGGAAGGCAAAA
TGCCGCAAAAAAGGGAATAAGGGCGACACGGAAATGTTGAATACTCATACTCTTCCTTTTTCAATATT
ATTGAAGCATTTATCAGGGTTATTGTCTCATGAGCGGATACATATTTGAATGTATTTAGAAAAATAAA
CAAATAGGGGTTCCGCGCACATTTCCCCGAAAAGTGCCACCTGACGTCTAAGAAACCATTATTATCAT
GACATTAACCTATAAAAATAGGCGTATCACGAGGCCCTTTCGTCTCGCGCGTTTCGGTGATGACGGTG
AAAACCTCTGACACATGCAGCTCCCGGAGACGGTCACAGCTTGTCTGTAAGCGGATGCCGGGAGCAGA
CAAGCCCGTCAGGGCGCGTCAGCGGGTGTTGGCGGGTGTCGGGGCTGGCTTAACTATGCGGCATCAGA
GCAGATTGTACTGAGAGTGCACCATATGCGGTGTGAAATACCGCACAGATGCGTAAGGAGAAAATACC
GCATCAGGCTAATACGACTCACTATAGGGACCAAGGGGAAAACCAAGATTAATCCTCTTCTTTTTTCT
TAAACTAATTCTGATTCTTGATATGTTTAGATTTCCTCGCCATCGATCCCGCGGTCTATTTCTgcTCA
GGCACCGGGCTTGCGGGTCATGCACCAGGTGCGCGGTCCTTCGGGCACCTCGACGTCGGCGGTGACGG
TGAAGCCGAGCCGCTCGNAGAAGGGGAGGTTGCGGGGCGCGGAGGTCTCCAGGAAGGCGGGCACCCCG
GCGCGCTCGGCCGCCTCCACTCCGGGGAGCACGACGGCGCTGCCCAGACCCTTGCCCTGGTGGTCGGG
CGAGACTCCGACGGTGGCCAGGAACCACGCGGGCTCCTTGGGCCGGTGCGGCGCCAGGAGGCCTTCCA
TCTGTTGCTGCGCGGCCAGCCGGGAACCGCTCAACTCGGCCATGCGCGGGCCGATCTCGGCGAACACC
GCCCCCGCTTCGACGCTCTCCGGCGTGGTCCAGACCGCCACCGCGGCGCCGTCGTCCGCGACCCACAC
CTTGCCGATGTCGAGCCCGACGCGCGTGAGGAAGAGTTCTTGCAGCTCGGTGACCCGCTCGATGTGGC
GGTCCGGATCGACGGTGTGGCGCGTGGCGGGGTAGTCGGCGAACGCGGCGGCGAGGGTGCGTACGGCC
CTGGGGACGTCGTCGCGGGTGGCGAGGCGCACCGTGGGCTTGTACTCGGTCATTGGGCCAGGATTCTC
CTCGACGTCACCGCATGTTAGCAGACTTCCTCTGCCCTCTCCCTTGTGGTACACGAAGTTTTCCATTC
TATTTCTGTTGGCCACCACGATGGTCAGCAGCTTCCACACCACCACACTCAGCAGAATGATCAGGATC
AAGCCCAGAGATCCCAGACAAATGGCGATGATGCTCAGCACGGAGGTGGTGGTGGCGCTTGTGATGGC
GCTCAGGTAGGTGTCGCTTTGAGCCAGGTGCTGCAGGGCATCGCTCAGGGACTTGTTCACGGCGGCCA
GGTTCTGAGAGATATCCAGAGGGTCGATGCTCAGGATCTGGCTGCTTTCCAGCTTAATGGTGCTGTTG
TAGGTCACGTTGGCCAGCTGTGTGATGGTGAACCGCAGGTTATCCAGCTGCAGGGAGACGCACTTATA
CATATCGATCACTGTCACGGGGGAGCTGCTAGGCTGCAAAATCACAGCGGCTGGCTGCATGCATTTAC
ACAGCATGCTCCGGCAGTTGGCGTAAACGATGCCGTCGAACAGCACGAATCTGGTCAGAAAAGAGCCC
ACCACTGGGCTGAAGGTACATCTTGTCAGGTTGCCTTGCAGGCAGGCCATTGTGTCGTCAGACAGCAC
TTGGGCGTCGTTGTATCTGCAGTACACTGTATTAGGGGTGATGGTGCACTGAGAGGCAGGATAAGCCT
GGATCAGGCTGCCGGTCACCATCACTCTGGTTGGCAGCTGGGCCATCACCTCCTGGTTGTTGATAAAA
GCGCTGATTGTAGCCAGGTCGATGATCTGGGTGGCAGGCTGGACTGTCAGTGTAGGCAGCTCGATCTT
GATGACCATCTGCATGTATGTCAGGTCCAGGCCCACGATCTGTCCTGTCAGCAGGCCGGAGCTCAGCA
GCTCGGCGGCGCTGATCTGGGTGTTGAAGCTCTTCTCGACCACTGTAGGCAGGGTAGAGCCGAGCAGG
ATTCTCAGGGCCTGGATGGTGATAGGGCTCAGGGGGGGGTTGGTGATCTGGTTGTGGAAGATTGTGGT
CAGTTCAGTCAGGTACAGGTTCAGGATGCTTCCGATGATGGCGTCCTGAGCTTTACAATTAGCAGCTG
TGATGGCAGGAGACACCACGCTATTGATATGATCCTGCACGGCCTGCACGGCGGTGCCCAGGCTCTGG
GTAGCTTGAACCACGTCGGCCACTGCGGCGTTGGTTTTCTGGATGGCGTTCTTCAGGTTCAGGATAGC
AGCGGCGTTCTCATTGGCCTTGACCAGGGCCACGGCGGCGGTCACCTGGGCGGCGGTGGCCACGCCGA
GAGCGGCCAGGCCGATCACCACTCCGGCGAACCTTCTCCGCCGTCTTGTGGGGATCAGCTGGTTCCGG
ATGGTTTCCAGGTTCTCGCCGATGGGCTGAAGCAGCTTTGTCACGGTGGCATTGTAGGAGCTGATGCT
GGTGATGTTGCAGCCGCTGATGGGAGAATCGATGGTAGGCATCAGCTTAACCACGATGAAGGCGCTAG
AGGCCTCGGTGTAGTACATCAGCTGGCGCACATTGGTAGGGATCACTCCAATCTGCATGAGAGCGGCG
GGGTCCAGAGAGCCGGCGCCGGCGAGCAGGCAGCTTACCACCAGAAATTGTATGATTGTGCCCATcaA
ATTCGCTTTATGATAACAATCTGTGATTGTCACCATAAGCAGCCACAATAAAATAAAAGGAAACACGG
ACACCCAAAGTAGTCGGTTCCGCCACGGACTTGCGCGTTACGACAGGCCAATCACTGGTTTGTGACCA
CCTGCTCCGAGGTTGGGATTAGCCGCATTCAGGGGCCGGAGGATTCTTATGTAGCTCAATAGGCTCTT
CACACCTTGTTCACAACTAGCGTCCCATGGCGTTAGCCATAGGTAGGCCGCCAACGCAGCCTGGACCA
CCGTCACCGGTGAGGGATGTCCAGACTCATCAGCCTAAGCTACACTCTGGGGTTGAGTGCTGAGCGCA
ACGCATCGAAGATTCCGAGGTGGTACTGGGCTTCTCGAAGTACATAAGCGGATAACGGATCCGTCGCT
TTCAACCACGCAAGCAGTCTATACGACATCACCGGGGAAACAGAAGTGCTTGTTCGTGGTGGTACTGG
TTTGTACCCCCTTCTATTGAACTTGGTTTTGTGCGTCTAAGTTACGGGAAGGGAGTATAAAACAGGCG
TACAAGGGTACCGCAATACCGGAGTACTAGCCGCCACGTGGGCCTCTGGGGTGGGTACAACCCCAGAG
CTGTTTTAAGCTAACTACTCTGACTCGAGGTCGGTCACGAAGGCCATAGGTTCCGGACCTATGTAATC
TCGAGGGTCTTCGATGATTTGAGTCACCACTTCATTTTCCCCTTGGTGGGTCGGCATGGCATCTCCAC
CTCCTCGCGGTCCGACCTGGGCATCCGAAGGAGGACGCACGTCCACTCGGATGGCTAAGGGAGCGGCC
GGGGATCCGGCTGCTAACAAAGCCCGAAAGGAAGCTGAGTTGGCTGCTGCCACCGCTGAGCAATAACT
AGCATAACCCCTTGGGGCCTCTAAACGGGTCTTGAGGGGTTTTTTGCTGAAAGGAGGAACTATATCCG
GAT
pIndel: IRES-SpikeΔCtV5-2A-Puro
CATGTGAGCAAAAGGCCAGCAAAAGGCCAGGAACCGTAAAAAGGCCGCGTTGCTGGCGTTTTTCCATA
GGCTCCGCCCCCCTGACGAGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCCTGTTCCGACCCTGCCGCT
TACCGGATACCTGTCCGCCTTTCTCCCTTCGGGAAGCGTGGCGCTTTCTCATAGCTCACGCTGTAGGT
ATCTCAGTTCGGTGTAGGTCGTTCGCTCCAAGCTGGGCTGTGTGCACGAACCCCCCGTTCAGCCCGAC
CGCTGCGCCTTATCCGGTAACTATCGTCTTGAGTCCAACCCGGTAAGACACGACTTATCGCCACTGGC
AGCAGCCACTGGTAACAGGATTAGCAGAGCGAGGTATGTAGGCGGTGCTACAGAGTTCTTGAAGTGGT
GGCCTAACTACGGCTACACTAGAAGAACAGTATTTGGTATCTGCGCTCTGCTGAAGCCAGTTACCTTC
GGAAAAAGAGTTGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTTCTACGGGGTCTG
ACGCTCAGTGGAACGAAAACTCACGTTAAGGGATTTTGGTCATGAGATTATCAAAAAGGATCTTCACC
TAGATCCTTTTAAATTAAAAATGAAGTTTTAAATCAATCTAAAGTATATATGAGTAAACTTGGTCTGA
CAGTTACCAATGCTTAATCAGTGAGGCACCTATCTCAGCGATCTGTCTATTTCGTTCATCCATAGTTG
CCTGACTCCCCGTCGTGTAGATAACTACGATACGGGAGGGCTTACCATCTGGCCCCAGTGCTGCAATG
ATACCGCGAGACCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGGGAAGCTAGAG
TAAGTAGTTCGCCAGTTAATAGTTTGCGCAACGTTGTTGCCATTGCTACAGGCATCGTGGTGTCACGC
TCGTCGTTTGGTATGGCTTCATTCAGCTCCGGTTCCCAACGATCAAGGCGAGTTACATGATCCCCCAT
GTTGTGCAAAAAAGCGGTTAGCTCCTTCGGTCCTCCGATCGTTGTCAGAAGTAAGTTGGCCGCAGTGT
TATCACTCATGGTTATGGCAGCACTGCATAATTCTCTTACTGTCATGCCATCCGTAAGATGCTTTTCT
GTGACTGGTGAGTACTCAACCAAGTCATTCTGAGAATAGTGTATGCGGCGACCGAGTTGCTCTTGCCC
GGCGTCAATACGGGATAATACCGCGCCACATAGCAGAACTTTAAAAGTGCTCATCATTGGAAAACGTT
CTTCGGGGCGAAAACTCTCAAGGATCTTACCGCTGTTGAGATCCAGTTCGATGTAACCCACTCGTGCA
CCCAACTGATCTTCAGCATCTTTTACTTTCACCAGCGTTTCTGGGTGAGCAAAAACAGGAAGGCAAAA
TGCCGCAAAAAAGGGAATAAGGGCGACACGGAAATGTTGAATACTCATACTCTTCCTTTTTCAATATT
ATTGAAGCATTTATCAGGGTTATTGTCTCATGAGCGGATACATATTTGAATGTATTTAGAAAAATAAA
CAAATAGGGGTTCCGCGCACATTTCCCCGAAAAGTGCCACCTGACGTCTAAGAAACCATTATTATCAT
GACATTAACCTATAAAAATAGGCGTATCACGAGGCCCTTTCGTCTCGCGCGTTTCGGTGATGACGGTG
AAAACCTCTGACACATGCAGCTCCCGGAGACGGTCACAGCTTGTCTGTAAGCGGATGCCGGGAGCAGA
CAAGCCCGTCAGGGCGCGTCAGCGGGTGTTGGCGGGTGTCGGGGCTGGCTTAACTATGCGGCATCAGA
GCAGATTGTACTGAGAGTGCACCATATGCGGTGTGAAATACCGCACAGATGCGTAAGGAGAAAATACC
GCATCAGGCTAATACGACTCACTATAGGGACCAAGGGGAAAACCAAGATTAATCCTCTTCTTTTTTCT
TAAACTAATTCTGATTCTTGATATGTTTAGATTTCCTCGCCATCGATCCCGCGGTCTATTTCTgcgcT
CAGGCACCGGGCTTGCGGGTCATGCACCAGGTGCGCGGTCCTTCGGGCACCTCGACGTCGGCGGTGAC
GGTGAAGCCGAGCCGCTCGTAGAAGGGGAGGTTGCGGGGCGCGGAGGTCTCCAGGAAGGCGGGCACCC
CGGCGCGCTCGGCCGCCTCCACTCCGGGGAGCACGACGGCGCTGCCCAGACCCTTGCCCTGGTGGTCG
GGCGAGACTCCGACGGTGGCCAGGAACCACGCGGGCTCCTTGGGCCGGTGCGGCGCCAGGAGGCCTTC
CATCTGTTGCTGCGCGGCCAGCCGGGAACCGCTCAACTCGGCCATGCGCGGGCCGATCTCGGCGAACA
CCGCCCCCGCTTCGACGCTCTCCGGCGTGGTCCAGACCGCCACCGCGGCGCCGTCGTCCGCGACCCAC
ACCTTGCCGATGTCGAGCCCGACGCGCGTGAGGAAGAGTTCTTGCAGCTCGGTGACCCGCTCGATGTG
GCGGTCCGGATCGACGGTGTGGCGCGTGGCGGGGTAGTCGGCGAACGCGGCGGCGAGGGTGCGTACGG
CCCTGGGGACGTCGTCGCGGGTGGCGAGGCGCACCGTGGGCTTGTACTCGGTCATTGGGCCAGGATTC
TCCTCGACGTCACCGCATGTTAGCAGACTTCCTCTGCCCTCTCCGGTGCTATCCAGGCCCAGCAGCGG
GTTCGGAATCGGTTTGCCACTGGTCATACAGCAAAGCATAATTGTCACCATTACTATGGCAATCAAGC
CAGCTATAAAACCTAGCCAAATGTACCATGGCCATTTTATATACTGCTCATACTTTCCAAGTTCTTGG
AGATCGATGAGAGATTCATTTAAATTCTTGGCAACCTCATTGAGGCGGTCAATTTCTTTTTGAATGTT
TACAACTGAAGCATTAATGCCAGAGATGTCACCTAAATCAACATCTGGTGATGTATGATTCTTAAAAT
ATTTATCTAACTCCTCCTTGAATGAGTCTAATTCAGGTTGCAAAGGATCATAAACTGTGTTGTTGACA
ATTCCTATTACAACATCACAGTTACCAGACACAAATGTGTTGTCTGTAGTAATGATTTGTGGTTCATA
AAAATTCCTTTGTGTTACAAACCAGTGTGTGCCATTTGAAACAAAGACACCTTCACGAGGAAAGTGTG
CTTTTCCATCATGACAAATGGCAGGAGCAGTTGTGAAGTTCTTTTCTTGTGCAGGGACATAAGTCACA
TGCAAGAAGACTACACCATGAGGTGCTGACTGAGGGAAGGACATAAGATGATAGCCCTTTCCACAAAA
ATCAACTCTTTTTGATTGTCCAAGTACACACTCTGACATTTTAGTAGCAGCAAGATTAGCAGAAGCTC
TGATTTCTGCAGCTCTAATTAATTGTTGAGTCACATATGTCTGCAAACTTTGAAGTCTGCCTGTGATC
AACCTATCAATTTGCACTTCAGCCTCAACTTTGTCAAGACGTGAAAGGATATCATTTAAAACACTTGA
AATTGCACCAAAATTGGAGCTAAGTTGTTTAACAAGCGTGTTTAAAGCTTGTGCATTTTGGTTGACCA
CATCTTGAAGTTTTCCAAGTGCACTTGCTGTGGAAGAAAGTGAGTCTTGAATTTTGCCAATAGCACTA
TTAAATTGGTTGGCAATCAATTTTTGGTTCTCATAGAGAACATTCTGTGTAACTCCAATACCATTAAA
CCTATAAGCCATTTGCATAGCAAATGGTATTTGTAATGCAGCACCTGCACCAAAGGTCCAACCAGAAG
TGATTGTACCCGCTAACAGTGCAGAAGTGTATTGAGCAATCATTTCATCTGTGAGCAAAGGTGGCAAA
ACAGTAAGGCCGTTAAACTTTTGTGCACAAATGAGGTCTCTAGCAGCAATATCACCAAGGCAATCACC
ATATTGTTTGATGAAGCCAGCATCTGCAAGTGTCACTTTGTTGAAAAGTAGATCTTCAATAAATGACC
TCTTGCTTGGTTTTGATGGATCTGGTAATATTTGTGAAAAATTAAAACCACCAAAATCTTTAATTGGT
GGTGTTTTGTAAATTTGTTTGACTTGTGCAAAAACTTCTTGGGTGTTTTTGTCTTGTTCAACAGCTAT
TCCAGTTAAAGCACGGTTTAATTGTGTACAAAAACTGCCATATTGCAACAAAAGATTGCTGCATTCAG
TTGAATCACCACAAATGTACATTGTACAATCTACTGATGTCTTGGTCATAGACACTGGTAGAATTTCT
GTGGTAACACTAATAGTAAAATTTGTGGGTATGGCAATAGAGTTATTAGAGTAAGCAACTGAATTTTC
TGCACCAAGTGACATAGTGTAGGCAATGATGGATTGACTAGCTACACTACGTGCCCGCCGAGGAGAAT
TAGTCTGAGTCTGATAACTAGCGCATATACCTGCACCAATGGGTATGTCACACTCATATGAGTTGTTG
ACATGTTCAGCCCCTATTAAACAGCCTGCACGTGTTTGAAAAACATTAGAACCTGTAGAATAAACACG
CCAAGTAGGAGTAAGTTGATCTGCATGAATAGCAACAGGGACTTCTGTGCAGTTAACATCCTGATAAA
GAACAGCAACCTGGTTAGAAGTATTTGTTCCTGGTGTTATAACACTGACACCACCAAAAGAACATGGT
GTAATGTCAAGAATCTCAAGTGTCTGTGGATCACGGACAGCATCAGTAGTGTCAGCAATGTCTCTGCC
AAATTGTTGGAAAGGCAGAAACTTTTTGTTAGACTCAGTAAGAACACCTGTGCCTGTTAAACCATTGA
AGTTGAAATTGACACATTTGTTTTTAACCAAATTAGTAGACTTTTTAGGTCCACAAACAGTTGCTGGT
GCATGTAGAAGTTCAAAAGAAAGTACTACTACTCTGTATGGTTGGTAACCAACACCATTAGTGGGTTG
GAAACCATATGATTGTAAAGGAAAGTAACAATTAAAACCTTCAACACCATTACAAGGTGTGCTACCGG
CCTGATAGATTTCAGTTGAAATATCTCTCTCAAAAGGTTTGAGATTAGACTTCCTAAACAATCTATAC
AGGTAATTATAATTACCACCAACCTTAGAATCAAGATTGTTAGAqTTCCAAGCTATAACGCAGCCTGT
AAAATCATCTGGTAATTTATAATTATAATCAGCAATCTTTCCAGTTTGCCCTGGAGCGATTTGTCTGA
CTTCATCACCTCTAATTACAAATGAATCTGCATAGACATTAGTAAAGCAGAGATCATTTAATTTAGTA
GGAGACACTCCATAACACTTAAAAGTGGAAAATGATGCGGAATTATATAGGACAGAATAATCAGCAAC
ACAGTTGCTGATTCTCTTCCTGTTCCAAGCATAAACAGATGCAAATCTGGTGGCGTTAAAAACTTCAC
CAAAAGGGCACAAGTTTGTAATATTAGGAAATCTAACAATAGATTCTGTTGGTTGGACTCTAAAGTTA
GAAGTTTGATAGATTCCTTTTTCTACAGTGAAGGATTTCAACGTACACTTTGTTTCTGAGAGAGGGTC
AAGTGCACAGTCTACAGCATCTGTAATGGTTCCATTTTCATTATATTTTAATAGAAAAGTCCTAGGTT
GAAGATAACCCACATAATAAGCTGCAGCACCAGCTGTCCAACCTGAAGAAGAATCACCAGGAGTCAAA
TAACTTCTATGTAAAGCAAGTAAAGTTTGAAACCTAGTGATGTTAATACCTATTGGCAAATCTACCAA
TGGTTCTAAAGCCGAAAAACCCTGAGGGAGATCACGCACTAAATTAATAGGCGTGTGCTTAGAATATA
TTTTAAAATAACCATCAATATTCTTAAACACAAATTCCCTAAGATTTTTGAAATTACCCTGTTTTCCT
TCAAGGTCCATAAGAAAAGGCTGAGAGACATATTCAAAAGTGCAATTATTCGCACTAGAATAAACTCT
GAACTCACTTTCCATCCAACTTTTGTTGTTTTTGTGGTAATAAACACCCAAAAATGGATCATTACAAA
ATTGAAATTCACAGACTTTAATAACAACATTAGTAGCGTTATTAACAATAAGTAGGGACTGGGTCTTC
GAATCTAAAGTAGTACCAAAAATCCAGCCTCTTATTATGTTAGACTTCTCAGTGGAAGCAAAATAAAC
ACCATCATTAAATGGTAGGACAGGGTTATCAAACCTCTTAGTACCATTGGTCCCAGAGACATGTATAG
CATGGAACCAAGTAACATTGGAAAAGAAAGGTAAGAACAAGTCCTGAGTTGAATGTAAAACTGAGGAT
CTGAAAACTTTGTCAGGGTAATAAACACCACGTGTGAAAGAATTAGTGTATGCAGGGGGTAATTGAGT
TCTGGTTGTAAGATTAACACACTGACTAGAGACTAGCGGCAATAAAACAAGAAAAACAAACATAATTC
GCTTTATGATAACAATCTGTGATTGTCACCATAAGCAGCCACAATAAAATAAAAGGAAACACGGACAC
CCAAAGTAGTCGGTTCCGCCACGGACTTGCGCGTTACGACAGGCCAATCACTGGTTTGTGACCACCTG
CTCCGAGGTTGGGATTAGCCGCATTCAGGGGCCGGAGGATTCTTATGTAGCTCAATAGGCTCTTCACA
CCTTGTTCACAACTAGCGTCCCATGGCGTTAGCCATAGGTAGGCCGCCAACGCAGCCTGGACCACCGT
CACCGGTGAGGGATGTCCAGACTCATCAGCCTAAGCTACACTCTGGGGTTGAGTGCTGAGCGCAACGC
ATCGAAGATTCCGAGGTGGTACTGGGCTTCTCGAAGTACATAAGCGGATAACGGATCCGTCGCTTTCA
ACCACGCAAGCAGTCTATACGACATCACCGGGGAAACAGAAGTGCTTGTTCGTGGTGGTACTGGTTTG
TACCCCCTTCTATTGAACTTGGTTTTGTGCGTCTAAGTTACGGGAAGGGAGTATAAAACAGGCGTACA
AGGGTACCGCAATACCGGAGTACTAGCCGCCACGTGGGCCTCTGGGGTGGGTACAACCCCAGAGCTGT
TTTAAGCTAACTACTCTGACTCGAGGTCGGTCACGAAGGCCATAGGTTCCGGACCTATGTAATCTCGA
GGGTCTTCGATGATTTGAGTCACCACTTCATTTTCCCCTTGGTGGGTCGGCATGGCATCTCCACCTCC
TCGCGGTCCGACCTGGGCATCCGAAGGAGGACGCACGTCCACTCGGATGGCTAAGGGAGCGGCCGGGG
ATCCGGCTGCTAACAAAGCCCGAAAGGAAGCTGAGTTGGCTGCTGCCACCGCTGAGCAATAACTAGCA
TAACCCCTTGGGGCCTCTAAACGGGTCTTGAGGGGTTTTTTGCTGAAAGGAGGAACTATATCCGGAT
pIndel: IRES-mCh-2A-Puro
CATGTGAGCAAAAGGCCAGCAAAAGGCCAGGAACCGTAAAAAGGCCGCGTTGCTGGCGTTTTTCCATA
GGCTCCGCCCCCCTGACGAGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCCTGTTCCGACCCTGCCGCT
TACCGGATACCTGTCCGCCTTTCTCCCTTCGGGAAGCGTGGCGCTTTCTCATAGCTCACGCTGTAGGT
ATCTCAGTTCGGTGTAGGTCGTTCGCTCCAAGCTGGGCTGTGTGCACGAACCCCCCGTTCAGCCCGAC
CGCTGCGCCTTATCCGGTAACTATCGTCTTGAGTCCAACCCGGTAAGACACGACTTATCGCCACTGGC
AGCAGCCACTGGTAACAGGATTAGCAGAGCGAGGTATGTAGGCGGTGCTACAGAGTTCTTGAAGTGGT
GGCCTAACTACGGCTACACTAGAAGAACAGTATTTGGTATCTGCGCTCTGCTGAAGCCAGTTACCTTC
GGAAAAAGAGTTGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTTCTACGGGGTCTG
ACGCTCAGTGGAACGAAAACTCACGTTAAGGGATTTTGGTCATGAGATTATCAAAAAGGATCTTCACC
TAGATCCTTTTAAATTAAAAATGAAGTTTTAAATCAATCTAAAGTATATATGAGTAAACTTGGTCTGA
CAGTTACCAATGCTTAATCAGTGAGGCACCTATCTCAGCGATCTGTCTATTTCGTTCATCCATAGTTG
CCTGACTCCCCGTCGTGTAGATAACTACGATACGGGAGGGCTTACCATCTGGCCCCAGTGCTGCAATG
ATACCGCGAGACCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGGGAAGCTAGAG
TAAGTAGTTCGCCAGTTAATAGTTTGCGCAACGTTGTTGCCATTGCTACAGGCATCGTGGTGTCACGC
TCGTCGTTTGGTATGGCTTCATTCAGCTCCGGTTCCCAACGATCAAGGCGAGTTACATGATCCCCCAT
GTTGTGCAAAAAAGCGGTTAGCTCCTTCGGTCCTCCGATCGTTGTCAGAAGTAAGTTGGCCGCAGTGT
TATCACTCATGGTTATGGCAGCACTGCATAATTCTCTTACTGTCATGCCATCCGTAAGATGCTTTTCT
GTGACTGGTGAGTACTCAACCAAGTCATTCTGAGAATAGTGTATGCGGCGACCGAGTTGCTCTTGCCC
GGCGTCAATACGGGATAATACCGCGCCACATAGCAGAACTTTAAAAGTGCTCATCATTGGAAAACGTT
CTTCGGGGCGAAAACTCTCAAGGATCTTACCGCTGTTGAGATCCAGTTCGATGTAACCCACTCGTGCA
CCCAACTGATCTTCAGCATCTTTTACTTTCACCAGCGTTTCTGGGTGAGCAAAAACAGGAAGGCAAAA
TGCCGCAAAAAAGGGAATAAGGGCGACACGGAAATGTTGAATACTCATACTCTTCCTTTTTCAATATT
ATTGAAGCATTTATCAGGGTTATTGTCTCATGAGCGGATACATATTTGAATGTATTTAGAAAAATAAA
CAAATAGGGGTTCCGCGCACATTTCCCCGAAAAGTGCCACCTGACGTCTAAGAAACCATTATTATCAT
GACATTAACCTATAAAAATAGGCGTATCACGAGGCCCTTTCGTCTCGCGCGTTTCGGTGATGACGGTG
AAAACCTCTGACACATGCAGCTCCCGGAGACGGTCACAGCTTGTCTGTAAGCGGATGCCGGGAGCAGA
CAAGCCCGTCAGGGCGCGTCAGCGGGTGTTGGCGGGTGTCGGGGCTGGCTTAACTATGCGGCATCAGA
GCAGATTGTACTGAGAGTGCACCATATGCGGTGTGAAATACCGCACAGATGCGTAAGGAGAAAATACC
GCATCAGGCTAATACGACTCACTATAGGGACCAAGGGGAAAACCAAGATTAATCCTCTTCTTTTTTCT
TAAACTAATTCTGATTCTTGATATGTTTAGATTTCCTCGCCATCGATCCCGCGGTCTATTTCTgcTCA
GGCACCGGGCTTGCGGGTCATGCACCAGGTGCGCGGTCCTTCGGGCACCTCGACGTCGGCGGTGACGG
TGAAGCCGAGCCGCTCGTAGAAGGGGAGGTTGCGGGGCGCGGAGGTCTCCAGGAAGGCGGGCACCCCG
GCGCGCTCGGCCGCCTCCACTCCGGGGAGCACGACGGCGCTGCCCAGACCCTTGCCCTGGTGGTCGGG
CGAGACTCCGACGGTGGCCAGGAACCACGCGGGCTCCTTGGGCCGGTGCGGCGCCAGGAGGCCTTCCA
TCTGTTGCTGCGCGGCCAGCCGGGAACCGCTCAACTCGGCCATGCGCGGGCCGATCTCGGCGAACACC
GCCCCCGCTTCGACGCTCTCCGGCGTGGTCCAGACCGCCACCGCGGCGCCGTCGTCCGCGACCCACAC
CTTGCCGATGTCGAGCCCGACGCGCGTGAGGAAGAGTTCTTGCAGCTCGGTGACCCGCTCGATGTGGC
GGTCCGGATCGACGGTGTGGCGCGTGGCGGGGTAGTCGGCGAACGCGGCGGCGAGGGTGCGTACGGCC
CTGGGGACGTCGTCGCGGGTGGCGAGGCGCACCGTGGGCTTGTACTCGGTCATTGGGCCAGGATTCTC
CTCGACGTCACCGCATGTTAGCAGACTTCCTCTGCCCTCTCCCTTGTACAGCTCGTCCATGCCGCCGG
TGGAGTGGCGGCCCTCGGCGCGTTCGTACTGTTCCACGATGGTGTAGTCCTCGTTGTGGGAGGTGATG
TCCAACTTGATGTTGACGTTGTAGGCGCCGGGCAGCTGCACGGGCTTCTTGGCCTTGTAGGTGGTCTT
GACCTCAGCGTCGTAGTGGCCGCCGTCCTTCAGCTTCAGCCTCTGCTTGATCTCGCCCTTCAGGGCGC
CGTCCTCGGGGTACATCCGCTCGGAGGAGGCCTCCCAGCCCATGGTCTTCTTCTGCATTACGGGGCCG
TCGGAGGGGAAGTTGGTGCCGCGCAGCTTCACCTTGTAGATGAACTCGCCGTCCTGCAGGGAGGAGTC
CTGGGTCACGGTCACCACGCCGCCGTCCTCGAAGTTCATCACGCGCTCCCACTTGAAGCCCTCGGGGA
AGGACAGCTTCAAGTAGTCGGGGATGTCGGCGGGGTGCTTCACGTAGGCCTTGGAGCCGTACATGAAC
TGAGGGGACAGGATGTCCCAGGCGAAGGGCAGGGGGCCACCCTTGGTCACCTTCAGCTTGGCGGTCTG
GGTGCCCTCGTAGGGGCGGCCCTCGCCCTCGCCCTCGATCTCGAACTCGTGGCCGTTCACGGAGCCCT
CCATGTGCACCTTGAAGCGCATGAACTCCTTGATGATGGCCATGTTATCCTCCTCGCCCTTGCTCACC
ATcacacAATTCGCTTTATGATAACAATCTGTGATTGTCACCATAAGCAGCCACAATAAAATAAAAGG
AAACACGGACACCCAAAGTAGTCGGTTCCGCCACGGACTTGCGCGTTACGACAGGCCAATCACTGGTT
TGTGACCACCTGCTCCGAGGTTGGGATTAGCCGCATTCAGGGGCCGGAGGATTCTTATGTAGCTCAAT
AGGCTCTTCACACCTTGTTCACAACTAGCGTCCCATGGCGTTAGCCATAGGTAGGCCGCCAACGCAGC
CTGGACCACCGTCACCGGTGAGGGATGTCCAGACTCATCAGCCTAAGCTACACTCTGGGGTTGAGTGC
TGAGCGCAACGCATCGAAGATTCCGAGGTGGTACTGGGCTTCTCGAAGTACATAAGCGGATAACGGAT
CCGTCGCTTTCAACCACGCAAGCAGTCTATACGACATCACCGGGGAAACAGAAGTGCTTGTTCGTGGT
GGTACTGGTTTGTACCCCCTTCTATTGAACTTGGTTTTGTGCGTCTAAGTTACGGGAAGGGAGTATAA
AACAGGCGTACAAGGGTACCGCAATACCGGAGTACTAGCCGCCACGTGGGCCTCTGGGGTGGGTACAA
CCCCAGAGCTGTTTTAAGCTAACTACTCTGACTCGAGGTCGGTCACGAAGGCCATAGGTTCCGGACCT
ATGTAATCTCGAGGGTCTTCGATGATTTGAGTCACCACTTCATTTTCCCCTTGGTGGGTCGGCATGGC
ATCTCCACCTCCTCGCGGTCCGACCTGGGCATCCGAAGGAGGACGCACGTCCACTCGGATGGCTAAGG
GAGCGGCCGGGGATCCGGCTGCTAACAAAGCCCGAAAGGAAGCTGAGTTGGCTGCTGCCACCGCTGAG
CAATAACTAGCATAACCCCTTGGGGCCTCTAAACGGGTCTTGAGGGGTTTTTTGCTGAAAGGAGGAAC
TATATCCGGAT
Indel NP-mCh-2A-Blast insert in T7 promoter orientation
CATGTGAGCAAAAGGCCAGCAAAAGGCCAGGAACCGTAAAAAGGCCGCGTTGCTGGCGTTTTTCCATA
GGCTCCGCCCCCCTGACGAGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCCTGTTCCGACCCTGCCGCT
TACCGGATACCTGTCCGCCTTTCTCCCTTCGGGAAGCGTGGCGCTTTCTCATAGCTCACGCTGTAGGT
ATCTCAGTTCGGTGTAGGTCGTTCGCTCCAAGCTGGGCTGTGTGCACGAACCCCCCGTTCAGCCCGAC
CGCTGCGCCTTATCCGGTAACTATCGTCTTGAGTCCAACCCGGTAAGACACGACTTATCGCCACTGGC
AGCAGCCACTGGTAACAGGATTAGCAGAGCGAGGTATGTAGGCGGTGCTACAGAGTTCTTGAAGTGGT
GGCCTAACTACGGCTACACTAGAAGAACAGTATTTGGTATCTGCGCTCTGCTGAAGCCAGTTACCTTC
GGAAAAAGAGTTGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTTCTACGGGGTCTG
ACGCTCAGTGGAACGAAAACTCACGTTAAGGGATTTTGGTCATGAGATTATCAAAAAGGATCTTCACC
TAGATCCTTTTAAATTAAAAATGAAGTTTTAAATCAATCTAAAGTATATATGAGTAAACTTGGTCTGA
CAGTTACCAATGCTTAATCAGTGAGGCACCTATCTCAGCGATCTGTCTATTTCGTTCATCCATAGTTG
CCTGACTCCCCGTCGTGTAGATAACTACGATACGGGAGGGCTTACCATCTGGCCCCAGTGCTGCAATG
ATACCGCGAGACCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGGGAAGCTAGAG
TAAGTAGTTCGCCAGTTAATAGTTTGCGCAACGTTGTTGCCATTGCTACAGGCATCGTGGTGTCACGC
TCGTCGTTTGGTATGGCTTCATTCAGCTCCGGTTCCCAACGATCAAGGCGAGTTACATGATCCCCCAT
GTTGTGCAAAAAAGCGGTTAGCTCCTTCGGTCCTCCGATCGTTGTCAGAAGTAAGTTGGCCGCAGTGT
TATCACTCATGGTTATGGCAGCACTGCATAATTCTCTTACTGTCATGCCATCCGTAAGATGCTTTTCT
GTGACTGGTGAGTACTCAACCAAGTCATTCTGAGAATAGTGTATGCGGCGACCGAGTTGCTCTTGCCC
GGCGTCAATACGGGATAATACCGCGCCACATAGCAGAACTTTAAAAGTGCTCATCATTGGAAAACGTT
CTTCGGGGCGAAAACTCTCAAGGATCTTACCGCTGTTGAGATCCAGTTCGATGTAACCCACTCGTGCA
CCCAACTGATCTTCAGCATCTTTTACTTTCACCAGCGTTTCTGGGTGAGCAAAAACAGGAAGGCAAAA
TGCCGCAAAAAAGGGAATAAGGGCGACACGGAAATGTTGAATACTCATACTCTTCCTTTTTCAATATT
ATTGAAGCATTTATCAGGGTTATTGTCTCATGAGCGGATACATATTTGAATGTATTTAGAAAAATAAA
CAAATAGGGGTTCCGCGCACATTTCCCCGAAAAGTGCCACCTGACGTCTAAGAAACCATTATTATCAT
GACATTAACCTATAAAAATAGGCGTATCACGAGGCCCTTTCGTCTCGCGCGTTTCGGTGATGACGGTG
AAAACCTCTGACACATGCAGCTCCCGGAGACGGTCACAGCTTGTCTGTAAGCGGATGCCGGGAGCAGA
CAAGCCCGTCAGGGCGCGTCAGCGGGTGTTGGCGGGTGTCGGGGCTGGCTTAACTATGCGGCATCAGA
GCAGATTGTACTGAGAGTGCACCATATGCGGTGTGAAATACCGCACAGATGCGTAAGGAGAAAATACC
GCATCAGGCTAATACGACTCACTATAGGGACCAAGGGGAAAACCAAGATTAATCCTCTTCTTTTTTCT
TAAACTAATTCTGATTCTTGATATGTTTAGATTTCCTCGCCATCGATCCCGCGGTCTATTTCTggggc
TTAGCCCTCCCACACATAACCAGAGGGCAGCAATTCACGAATCCCAACTGCCGTCGGCTGTCCATCAC
TGTCCTTCACTATGGCTTTGATCCCAGGATGCAGATCGAGAAGCACCTGTCGGCACCGTCCGCAGGGG
CTCAAGATGCCCCTGTTCTCATTTCCGATCGCGACGATACAAGTCAGGTTGCCAGCTGCCGCAGCAGC
AGCAGTGCCCAGCACCACGAGTTCTGCACAAGGTCCCCCAGTAAAATGATATACATTGACACCAGTGA
AGATGCGGCCGTCGCTAGAGAGAGCTGCGCTGGCGACGCTGTAGTCTTCAGAGATGGGGATGCTGTTG
ATTGTAGCCGTTGCTCTTTCAATGAGGGTGGATTCTTCTTGAGACAAAGGCTTGGCCATTGGGCCAGG
ATTCTCCTCGACGTCACCGCATGTTAGCAGACTTCCTCTGCCCTCTCCCTTGTACAGCTCGTCCATGC
CGCCGGTGGAGTGGCGGCCCTCGGCGCGTTCGTACTGTTCCACGATGGTGTAGTCCTCGTTGTGGGAG
GTGATGTCCAACTTGATGTTGACGTTGTAGGCGCCGGGCAGCTGCACGGGCTTCTTGGCCTTGTAGGT
GGTCTTGACCTCAGCGTCGTAGTGGCCGCCGTCCTTCAGCTTCAGCCTCTGCTTGATCTCGCCCTTCA
GGGCGCCGTCCTCGGGGTACATCCGCTCGGAGGAGGCCTCCCAGCCCATGGTCTTCTTCTGCATTACG
GGGCCGTCGGAGGGGAAGTTGGTGCCGCGCAGCTTCACCTTGTAGATGAACTCGCCGTCCTGCAGGGA
GGAGTCCTGGGTCACGGTCACCACGCCGCCGTCCTCGAAGTTCATCACGCGCTCCCACTTGAAGCCCT
CGGGGAAGGACAGCTTCAAGTAGTCGGGGATGTCGGCGGGGTGCTTCACGTAGGCCTTGGAGCCGTAC
ATGAACTGAGGGGACAGGATGTCCCAGGCGAAGGGCAGGGGGCCACCCTTGGTCACCTTCAGCTTGGC
GGTCTGGGTGCCCTCGTAGGGGCGGCCCTCGCCCTCGCCCTCGATCTCGAACTCGTGGCCGTTCACGG
AGCCCTCCATGTGCACCTTGAAGCGCATGAACTCCTTGATGATGGCCATGTTATCCTCCTCGCCCTTG
CTCACCATTGTAGATTGTGGATTGCGCTGTTGGCACCGGCAGTCGCTTGTTGCTAACCCGTCCGGGCC
TATTTTTTTCTTTAAAGCCGATGGTAGGCAGGAACCTTATGGAATCCACTGTAGTTCCATCTAACTAA
TCAATTAGGTATGTCAATTTTAGATTGGGATCTGAATTGTCTAGATGTCAAGATCACCCAGTGCGGCA
TTCAGGTCATCTGTATGGATGGGAGCTCCAACTGCTGCGTTCTGGATGTCCAAGGCCACTTGTTCAAG
AGTTGGTCCAGTGCGTGGACCTGCAGATGTGTCGTCCTCTTCCTCCTCTTCCAATGGTGGCATCTCAT
CATCATGGGTGCCTGCATCAGCATATCTCTGCCTCAGTCGATCCGCTGCCATGTAACTCTCGAGTGTG
TCACCTGTTGCTGCAGCGGGCACCTGAGTTGTCCCAGTGACAGATGAGAACGGGTTGACTCCTCCCCT
GGTGTCTGCCCCTCGATTTGCTGTGGTCAATTTGGCAAGTGTATTTGCCATCTCGGTGCGTTCGGCTT
GAGTTAGACCGAGATCTTCTGCCATCCTCATGTCAACTGCACCCTGTTGTTTTCTTGCAGTTTCCATT
CCCAATTGGAAATATGTCTTGTTCATGTATGATCTGGAGAAAGCGTAGTTCCTCATGTTGACATCTAA
CACATAGCCTATTCCCATAGCATAGCTATATAGCAGTGGGTAGTTTGCTGCAGCAAAATCCATCAAAT
GTGGTGACTCCAATAGGGCCAAATATCGGGCCTGCTCACCAAGGGTCTGGTATAATGCCATGAGGGAC
TTTAGCTTTGTTAGCTCTCCTGAAAATGCAGCTAAAGCAAGTGTGGGCCATCTAGTTCCTAATGCATA
TTTTAGTGTCAAAAAGAAGCCTCCCATTCCACAATTCTCTATATACTTTCCAACATCCCCTACCATAG
CATAATACCTATTTGATACAAGGCTTTGTGCTCGGGCAAGCTGCAGTTCAAAGGTGAGGAAATGTCTG
ACCACCATTCCCTTCCGGATTACATTCTGGATTATTCGTCGAGCCTCCGGTTGCAGGAGATATCTCGG
GTTGATCCTGCCTTGCTGACGATATTTTTGCAGGCGTTTCTCAATAGAAGCAGCAGGTTGGTCTGGCG
CAGTCATGCACTTGCAAGTCACTATCCATGCCTGCATTAGGACACTGTAACACATATCTAAGAAAGTC
TCAATCTCATCCCATGCAGTTCCCTCGACTTCGGAATCAACGAAAGGTGTTGCATGGTTTAGTGTGTC
AGGTAGATCTTCTGCAAGTGCAGCATAGGCATTGATCTCTCCACGGCTCATACCTGAACGaGCATTGG
GGATTAAGCGGAATGATCCCTCCTCAAAGCCATCGATCTCTACCCTTTCGATATCAGCTTCTGGTGAC
TGGTCAGCTAATTTGACATGATTGAGCATTGTGGCTGATGGTAGCGAAAACATTGTGAGTAATGCTCC
AAAGCGATGGGAATCCCTTGCACCATTACTGAGAACAATCCGTAGGCAGAATAGAAGAAGCCGGGATC
TTAGCTCTGGGTTATTAGAGGTTAGTATAAATACCCTGATTACCGGTTTTAGTGTTGTAGGTGGGATT
GTACCTTCCTCACTCTGATCTTGCAGTTCTTGAGTGAGCGTGAATCGCTCATATGCTTTAAGCACGGA
TGACATTTTTGTGTCGGGGACGAAAAATTGCCCAAACTTGATAGGTCCTTATTGAACTACTCTGACTC
GAGGTCGGTCACGAAGGCCATAGGTTCCGGACCTATGTAATCTCGAGGGTCTTCGATGATTTGAGTCA
CCACTTCATTTTCCCCTTGGTGGGTCGGCATGGCATCTCCACCTCCTCGCGGTCCGACCTGGGCATCC
GAAGGAGGACGCACGTCCACTCGGATGGCTAAGGGAGCGGCCGGGGATCCGGCTGCTAACAAAGCCCG
AAAGGAAGCTGAGTTGGCTGCTGCCACCGCTGAGCAATAACTAGCATAACCCCTTGGGGCCTCTAAAC
GGGTCTTGAGGGGTTTTTTGCTGAAAGGAGGAACTATATCCGGAT
NP primers (use minigenome vector)to make NP: NP-P intergenic region and P UTR
1. mCherry-NP-top
CCTCCTCGCCCTTGCTCACCATTGTAGATTGTGGATTGCGC
2. NP-genomic prom-low
CCGACCTCGAGTCAGAGTAGTTCAATAAGGACCTATCAAGTTTGGG
PCR primers to remove IRES from InDel-IRES-mCherry-2A-Blast vector for
subsequent insertion of NP
3. InDel-delta IRES-mCherry rev
ATGGTGAGCAAGGGCGAGGAG
4. InDel-delta IRES-mCherry for
ACTACTCTGACTCGAGGTCGG
Run PCR using Phusion polymerase and re-ligate.
PCR primers to make InDel-NP-mCherry-2A-Blast
5. NP-genomic prom-top
CCCAAACTTGATAGGTCCTTATTGAACTACTCTGACTCGAGGTCGG
6. mCherry-NP-low
GCGCAATCCACAATCTACAATGGTGAGCAAGGGCGAGGAGG

1. A product derived from the PIV5 genome comprising the 3′ replication promoter of the PIV5 genome, duplicated and in the opposite orientation together with some of the L gene sequence.

2. The product of claim 1, obtainable by:
(i) passaging a wild type PIV5 in a cell; or
(ii) passaging PIV5ΔF in a cell which expresses the PIV F protein.

3. The product of claim 2, wherein the passaging is done at a high multiplicity of infection.

4. A PIV5 derived Indel vector comprising a nucleic acid sequence which, relative to a wild-type PIV5 genome, lacks the NP, P/V, M, F, SH, HN and/or L genes.

5. The PIV5 Indel vector of claim 4, wherein the Indel vector retains or comprises the 3′ Le and 5′ Tr sequences of PIV5 viral genome.

6. The PIV5 derived Indel vector of claim 4, wherein the vector further comprises a heterologous sequence for expression.

7. The PIV5 derived Indel vector of claim 6, wherein the heterologous sequences encodes or provides any one or more of the following categories of protein:
(i) antigens (including viral and/or bacterial antigens);
(ii) tumour specific antigens;
(iii) multivalent CTL antigens;
(iv) recombinant proteins (for expression);
(v) components of the immune system;
(vi) immunomodulatory compounds;
(vii) antibodies (including fragments and/or parts thereof); and
(viii) cytokines.

8. (canceled)

9. A vector derived from the parainfluenza virus 5 (PIV5) genome
wherein relative to a wild-type PIV5 genome, the vector comprises one or more deleted or functionally deleted wild-type PIV5 genes.

10. (canceled)

11. The vector of claim 9, wherein the vector further comprises a heterologous sequence for expression.

12. The vector of claim 11, wherein the heterologous sequence encodes or provides any one or more of the following categories of protein:
(i) antigens (including viral and/or bacterial antigens);
(ii) tumour specific antigens;
(iii) multivalent CTL antigens;
(iv) recombinant proteins (for expression);
(v) components of the immune system;
(vii) immunomodulatory compounds;
(viii) antibodies (including fragments and/or parts thereof);
(ix) cytokines.

13. The vector of claim 9, wherein the vector comprises the deletion or functional deletion of:
the F gene; and/or
the M gene; and/or
the HN gene.

14. The vector of claim 9, wherein the vector lacks a functional copy of the PIV5 F gene.

15. The vector of claim 9, wherein the vector lacks a functional copy of the PIV5 F and M genes.

16. The vector of claim 9, wherein the vector lacks functional copies of the PIV5 M, F and HN genes.

17. A method of (i) of replicating a PIV5 vector, said method comprising, contacting a PIV5 vector with a helper cell.

18. The method of claim 17, wherein the helper cell is permissive to a PIV5 virus.

19. The method of claim 17, wherein the helper cell is a cell which expresses the gene or genes deleted or functionally deleted from the vector.

20. A cell modified to express the:
(i) PIV5 F protein; and/or
(ii) the PIV5 M protein; and/or
(iii) the PIV5 HN protein.

21. A method of modulating, improving or augmenting an immune response to an antigen or vaccine, said method comprising immunising a subject with the vaccine or antigen and a vector or product according to claim 11.

22. An immunogenic composition comprising an antigen and a vector or product according to claim 11.

23. A method of treating or preventing:
acute or chronic diseases and infections; or
cancer;
said method comprising administering a subject in need thereof, a therapeutically effective amount of a vector or product according to claim 11.

24. A method of modulating gene expression in a cell, said method comprising contacting the cell with a product according to claim 1.

25. The method of claim 24, wherein one or more of the genes listed in Table 1 can be (directly or indirectly) modulated:
TABLE 1

| No | Gene |
|---|---|
| 1 | KCNJ18 |
| 2 | FAM45A |
| 3 | RSAD2 |
| 4 | CYP2D6 |
| 5 | IFNL2 |
| 6 | TNFSF12-TN |
| 7 | IFNL1 |
| 8 | C10orf32-AS |
| 9 | IFIT2 |
| 10 | CH25H |
| 11 | IFNL4 |
| 12 | DHX58 |
| 13 | OASL |
| 14 | IFIT1 |
| 15 | IFNB1 |
| 16 | HCAR2 |
| 17 | OAS2 |
| 18 | FSBP |
| 19 | SAMD9L |
| 20 | IFIH1 |
| 21 | XAF1 |
| 22 | IFIT3 |
| 23 | MX1 |
| 24 | HCAR |
| 25 | GBP1 |
| 26 | LRP2 |
| 27 | TAC3 |
| 8 | MX2 |
| 29 | TNFSF10 |
| 30 | IL6 |
| 31 | CMPK2 |
| 32 | GBP4 |
| 33 | IDO1 |
| 34 | CCL5 |
| 35 | SAMD9 |
| 36 | DDX58 |
| 37 | IFI27 |
| 38 | IFI44 |
| 39 | TNFSF13B |
| 40 | ISG15 |
| 41 | HERC5 |
| 42 | FAM65B |
| 43 | IFI27 |


SEQUENCE LISTING (14 sequences)

SEQ ID NO: 1 - DNA, 16687 bp, synthetic construct
  source 1..16687: mol_type = other DNA
catgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggctaatacgactcactatagggaccaaggggaaaatgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacgctcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattactcacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacacctttcgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgacgaataatccagaatgtaatccggaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaactagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaacaagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctgtcactgggacaactcaggtgcccgctgcagcaacaggtgacacactcgagagttacatggcagcggatcgactgaggcagagatatgctgatgcaggcacccatgatgatgagatgccaccattggaagaggaggaagaggacgacacatctgcaggtccacgcactggaccaactcttgaacaagtggccttggacatccagaacgcagcagttggagctcccatccatacagatgacctgaatgccgcactgggtgatcttgacatctagacaattcagatcccaatctaaaattgacatacctaattgattagttagatggaactacagtggattccataaggttcctgcctaccatcggctttaaagaaaaaaataggcccggacgggttagcaacaagcgactgccggtgccaacagcgcaatccacaatctacaatggatcccactgatctgagcttctccccagatgagatcaataagctcatagagacaggcctgaatactgtagagtattttacttcccaacaagtcacaggaacatcctctcttggaaagaatacaataccaccaggggtcacaggactactaaccaatgctgcagaggcaaagatccaagagtcaactaaccatcagaagggctcagttggtgggggtgcaaaaccaaagaaaccgcgaccaaaaattgccattgtgccagcagatgacaaaacagtgcccggaaagccgatcccaaaccctctattaggtctggactccaccccgagcacccaaactgtgcttgatctaagtgggaaaacattaccatcaggatcctataagggggttaagcttgcgaaatttggaaaagaaaatctgatgacacggttcatcgaggaacccagagagaatcctatcgcaaccagttcccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagacactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagattacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacactacctgaggcagccatgccatccatcagcattcccgcagaccccaccaatccacgtcaatcaataaaagcgttcccaattgtgatcaacagtgatgggggtgagaaaggccgcttggttaaacaactacgcacaacctacttgaatgacctagatactcatgagccactggtgacattcataaatacctatggattcatctacgaacaggatcgggggaataccattgtcggagaggatcaacttgggaagaaaagagaggctgtgaccgctgcaatggttacccttggatgtgggcctaatctaccatcattagggaatgtcctgggacaactgagggaattccaggtcactgttaggaagacatccagcaaagcggaagagatggtctttgaaattgttaagtatccgagaatatttcggggtcatacattaatccagaaaggactagtctgtgtctccgcagaaaaatttgttaagtcaccagggaaaatacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatacccctgacaaggagggaagaggatgtcttgtatcagtatggctgcatgtatgcaacatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaactaaatcactgcctaacctgacggagctggaatgactgacctctaatcgagactacaccgccgcaaactataggtgggtggtacctcagtgattaatcttgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaaggaccgaacctagtattgaaagaaccgtctcggtcaatctaggtaatcgagctgataccgtctcggaaagctcaaatcatgctgcctgatccggaagatccggaaagcaagaaagctacaaggagagcaggaaacctaattatctgcttcctattcatcttctttctgtttgtaaccttcattgttccaactctaagacacttgctgtcctaacacctgctataggctatccactgcatcatctctcctgccatacttcctactcacatcatatctattttaaagaaaaaataggcccgaacactaatcgtgccggcagtgccactgcacacacaacactacacatacaatacactacaatggttgcagaagatgcccctgttagggccacttgccgagtattatttcgaacaacaactttaatctttctatgcacactactagcattaagcatctctatcctttatgagagtttaataacccaaaagcaaatcatgagccaagcaggctcaactggatctaattctggattaggaagtatcactgatcttcttaataatattctctctgtcgcaaatcagattatatataactctgcagtcgctctacctctacaattggacactcttgaatcaacactccttacagccattaagtctcttcaaaccagtgacaagctagaacagaactgctcgtggagtgctgcactgattaatgataatagatacattaatggcatcaatcagttctatttttcaattgctgagggtcgcaatctgacacttggcccacttcttaatatgcctagtttcattccaactgccacgacaccagagggctgcaccaggatcccatcattctcgctcactaagacacactggtgttatacacacaatgttatcctgaatggatgccaggatcatgtatcctcaaatcaatttgtttctatgggaatcattgaacccacttctgccgggtttccattctttcgaaccctaaagactctatatctcagcgatggggtcaatcgtaagagctgctctatcagtacagttccggggggttgtatgatgtactgttttgtttctactcaaccagagagggatgactacttttctgccgctcctccagaacaacgaattattataatgtactataatgatacaatcgtggagcgcataattaatccacccggggtactagatgtatgggcaacattgaacccaggaacaggaagcggggtatattatttaggttgggtgctctttccaatatatggcggcgtgattaaaggtacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcgacaggatctaaccaatgagtgtttagttctgcccttttctaatgatcaggtgcttatgggtgctgaagggagattatacatgtatggtgactcggtgtattactatcaaagaagcaatagttggtggcctatgaccatgctgtataaggtaaccataacattcactaatggtcagccatctgctatatcagctcagaatgtgcccacacagcaggtccctagacctgggacaggagactgctctgcaaccaatagatgtcccggtttttgcttgacaggagtgtatgccgatgcctggttactgaccaacccttcgtctaccagtacatttggatcagaagcaaccttcactggttcttatctcaacacagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagccttcaggtctgacccagccaatcaaagcattataccagaccatggcctaccatcggctttaaagaaaaaaataggcccggacgggttagcaacaagcggcggccgcaatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaggcggccgcctaaggtcgactcatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgaccttgacgatttaggtccattacataatcaaaattggaatcagatagcacatgaagagtctaacttagctcaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctgctcaaaaattgggacaagttagttaaagaatcatgttcagtaatcaatgcaggtacttcccagtgcattcagaatctcagctatggactgacaggtcgtgggaacctctttacacgatcacgtgaactctctggtgaccgcagggatattgatcttaagacagttgtggcagcatggcatgactcagactggaaaagaataagtgatttttggattatgatcaaattccagatgagacaattaattgttaggcaaacagatcataatgattctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaagaggaactaatagtttcacccaggatgaattaagaactgtgacaactaatttgatatccccattccaagatctgaccccagatcttacggctgaattgctctgtataatgaggctttggggacaccccatgctcactgccagtcaagctgcaggaaaggtacgcgagtctatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgaaataagctatgaattcacacttaagcattggaaggaagtctctcttataaaattcaagaaatgttttgatgcagacgcaggtgaggaactcagtatatttatgaaagataaggcaattagtgccccaaaacaagactggatgagtgtgtttagaagaagcctaatcaaacagcgccatcagcatcatcaggtccccctaccaaatccattcaatcgacggctgttgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaagtcaactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctgtgtcaaaaggcttggacaatgatatctatcgctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgcacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttttgccatcccagttaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccctccaactacagctcttaagaggcacacccaacaagctctgatggaactcagtacgaatccaatgttacgtggcatattctctgacaatgcacaggcagaagaaaataaccttgctaggtttctcctggatagggaggtgatctttccgcgtgtagctcacatcatcattgagcaaaccagtgtcgggaggagaaaacagattcaaggatatttggattcaactagatcgataatgaggaaatcactagaaattaagcccttatccaataggaagcttaatgaaatactggattacaacatcaattacctagcttacaatttggcattactcaagaatgctattgaacctccgacttatttgaaggcaatgacacttgaaacatgtagcatcgacattgcaaggaacctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcttaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaatcttagaaacatggaataatcccccaatcaatagaacattcgaagaatctaccctacatttgcacactggtgcatcatgttgtgtccgacctgtggactcctgcattctctcagaagcattaacagtcaagccacatattacagtaccgtacagcaataaatttgtatttgatgaggacccgctatctgaatatgaaactgcaaaactggaatcgttatcattccaagcccaattaggcaacattgatgctgtagatatgacaggtaaattaacattattgtcccaattcactgcaaggcagattatcaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggcacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgctgcaggtaccttgagcgattaaagctaccacaaggtgatcatttatatattggagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaacgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctgatccagctaatcatgaggtttatttaatttgtatccttgccaacaactttgcattccagactgtctcgcaggcaacaggaatggcgatgactttaactgatcaagggtttactttgatatcacctgaaagaataaatcagtattgggatggtcacttgaagcaagaacgtatcgtagcagaagcaattgataaggtggttctaggagaaaatgctctatttaattcgagtgataatgaattaattctcaaatgtggagggacaccaaatgcacggaatctcatcgatatcgagccagtcgcaactttcatagaatttgaacaattgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattgcacaattaactgcaggctacattaggaaattgattgagggggattgcaatatcgatctaaccagacctatccaaaagcaaatctggaaagcattaggttgtgtagtctattgtcacgatccaatggatcaaagggagtcaacagagtttattgatataaatattaatgaagaaatagaccgcgggatcgatggcgaggaaatctaaacatatcaagaatcagaattagtttaagaaaaaagaagaggattaatcttggttttccccttggtgggtcggcatggcatctccacctcctcgcggtccgacctgggcatccgaaggaggacgcacgtccactcggatggctaagggagcggccggggatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 2 - DNA, 21445 bp, synthetic construct
  source 1..21445: mol_type = other DNA
catgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggctaatacgactcactatagggaccaaggggaaaatgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacgctcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattactcacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacacctttcgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgacgaataatccagaatgtaatccggaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaactagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaacaagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctgtcactgggacaactcaggtgcccgctgcagcaacaggtgacacactcgagagttacatggcagcggatcgactgaggcagagatatgctgatgcaggcacccatgatgatgagatgccaccattggaagaggaggaagaggacgacacatctgcaggtccacgcactggaccaactcttgaacaagtggccttggacatccagaacgcagcagttggagctcccatccatacagatgacctgaatgccgcactgggtgatcttgacatctagacaattcagatcccaatctaaaattgacatacctaattgattagttagatggaactacagtggattccataaggttcctgcctaccatcggctttaaagaaaaaaataggcccggacgggttagcaacaagcgactgccggtgccaacagcgcaatccacaatctacaatggatcccactgatctgagcttctccccagatgagatcaataagctcatagagacaggcctgaatactgtagagtattttacttcccaacaagtcacaggaacatcctctcttggaaagaatacaataccaccaggggtcacaggactactaaccaatgctgcagaggcaaagatccaagagtcaactaaccatcagaagggctcagttggtgggggtgcaaaaccaaagaaaccgcgaccaaaaattgccattgtgccagcagatgacaaaacagtgcccggaaagccgatcccagaccctctattaggtctggactccaccccgagcacccaaactgtgcttgatctaagtgggaaaacattaccatcaggatcctataagggggttaagcttgcgaaatttggaaaagaaaatctgatgacacggttcatcgaggaacccagagagaatcctatcgcaaccagtttccccatcgattttaagaggggcaggtataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagacactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagattacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacactacctgaggcagccatgccatccatcagcattcccgcagaccccaccaatccacgtcaatcaataaaagcgttcccaattgtgatcaacagtgatgggggtgagaaaggccgcttggttaaacaactacgcacaacctacttgaatgacctagatactcatgagccactggtgacattcataaatacctatggattcatctacgaacaggatcgggggaataccattgtcggagaggatcaacttgggaagaaaagagaggctgtgaccgctgcaatggttacccttggatgtgggcctaatctaccatcattagggaatgtcctgggacaactgagggaattccaggtcactgttaggaagacatccagcaaagcggaagagatggtctttgaaattgttaagtatccgagaatatttcggggtcatacattaatccagaaaggactagtctgtgtctccgcagaaaaatttgttaagtcaccagggaaaatacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatacccctgacaaggagggaagaggatgtcttgtatcagtatggctgcatgtatgcaacatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaactaaatcactgcctaacctgacggagctggaatgactgacctctaatcgagactacaccgccgcaaactataggtgggtggtacctcagtgattaatcttgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatggaccccaagggcagcctgagctggagaatcctgctgttcctgagcctggccttcgagctgagctacggcgacgtgcaggtggtggagagcggaggcggagtggtgcagccaggcggcagcctgagactgagctgcgctgcgagtggcttcaccttcagcagcttcgggatgcactgggtgaggcaggcacccggcaagggcctggagtgggtggcctacatcaacaccgacagcaccaccatctactacgcagacagcgtgaagggcaggttcaccatcagcagggacaacagcaagaacaccctgtacctgcagatgaactctttgcgggcagaagacactgccgtgtactactgcgccagtgctggcccctactatggctttgactactggggacagggcaccctggtgaccgtgagcagcgctagcaccaagggcctcagcgtgttccctctggcccccagcagcaagagcaccagcggcggaaccgccgccctgggctgcctggtgaaggactacttccccgagcccgtgaccgtgtcctggaacagcggcgctctgaccagcggagtgcacaccttccctgccgtgctgcagagcagcggcctgtactccctgagcagcgtggtgaccgtgcccagcagcagcctgggcacccagacctacatctgcaacgtgaaccacaagccctccaacaccaaggtggacaagaaggtggagcctaagagctgcgacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacaactccacctaccgcgtggtgagcgtgctgaccgtgctgcaccaggactggctgaacggcaaggagtacaagtgcaaggtgagcaacaaggccctgcccgctcccatcgagaagaccatcagcaaggccaagggccagcctcgggagcctcaggtgtacaccctgccccccagccgcgaagagatgaccaagaaccaggtgagcctgacctgcctggtgaagggcttctacccctccgacatcgccgtggagtgggagagcaacggccagcctgagaacaactacaagaccacccctcccgtgctggacagcgacggcagcttcttcctgtacagcaagctgaccgtggacaagtcccggtggcagcagggcaacgtgttcagctgcagcgtgatgcacgaggccctgcacaaccactacacccagaagagcctgagcctgagccccggacgcgcaaaaagatctagagagggcaggggaagtcttctaacatgcggggacgtggaggaaaatcccgggcctatggagaccgacaccctgctgctctgggtgctgctgctctgggtgcccggctccaccggagacatcgtgatgacccagagtccactgagcctgcccgtgacccccggtgagccagccagcatcagctgcaggagcagcaagagcctggtgcacagcaacggcatcacctacctgtactggtacgtgcagaagcccggacagagcccccagttgctcatttatcagatgagcagcctggcaagcggcgtgcccgacaggttcagcggctccggcagcggcaccgacttcagcctgaagatcagccgggtggaggccgaggacgtgggcgtgtactactgcggccaaattctggagctgcccttcaccttcggccagggaaccaaggtggagatcaagcggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaatgagcattccaccactcacgatctgatctcagtgagaaaaatcaacctgcaactcttggaacaagataagacagtcatccattagtaatttttaagaaaaaaacgataggaccgaacctagtattgaaagaaccgtctcggtcaatctaggtaatcgagctgataccgtctcggaaagctcaaatcatgctgcctgatccggaagatccggaaagcaagaaagctacaaggagagcaggaaacctaattatctgcttcctattcatcttctttctgtttgtaaccttcattgttccaactctaagacacttgctgtcctaacacctgctataggctatccactgcatcatctctcctgccatacttcctactcacatcatatctattttaaagaaaaaataggcccgaacactaatcgtgccggcagtgccactgcacacacaacactacacatacaatacactacaatggttgcagaagatgcccctgttagggccacttgccgagtattatttcgaacaacaactttaatctttctatgcacactactagcattaagcatctctatcctttatgagagtttaataacccaaaagcaaatcatgagccaagcaggctcaactggatctaattctggattaggaagtatcactgatcttcttaataatattctctctgtcgcaaatcagattatatataactctgcagtcgctctacctctacaattggacactcttgaatcaacactccttacagccattaagtctcttcaaaccagtgacaagctagaacagaactgctcgtggagtgctgcactgattaatgataatagatacattaatggcatcaatcagttctatttttcaattgctgagggtcgcaatctgacacttggcccacttcttaatatgcctagtttcattccaactgccacgacaccagagggctgcaccaggatcccatcattctcgctcactaagacacactggtgttatacacacaatgttatcctgaatggatgccaggatcatgtatcctcaaatcaatttgtttctatgggaatcattgaacccacttctgccgggtttccattctttcgaaccctaaagactctatatctcagcgatggggtcaatcgtaagagctgctctatcagtacagttccggggggttgtatgatgtactgttttgtttctactcaaccagagagggatgactacttttctgccgctcctccagaacaacgaattattataatgtactataatgatacaatcgtggagcgcataattaatccacccggggtactagatgtatgggcaacattgaacccaggaacaggaagcggggtatattatttaggttgggtgctctttccaatatatggcggcgtgattaaaggtacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcgacaggatctaaccaatgagtgtttagttctgcccttttctaatgatcaggtgcttatgggtgctgaagggagattatacatgtatggtgactcggtgtattactatcaaagaagcaatagttggtggcctatgaccatgctgtataaggtaaccataacattcactaatggtcagccatctgctatatcagctcagaatgtgcccacacagcaggtccctagacctgggacaggagactgctctgcaaccaatagatgtcccggtttttgcttgacaggagtgtatgccgatgcctggttactgaccaacccttcgtctaccagtacatttggatcagaagcaaccttcactggttcttatctcaacacagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagccttcaggtctgacccagccaatcaaagcattataccagaccatggcctaccatcggctttaaagaaaaaaataggcccggacgggttagcaacaagcggcggccgcaatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaggcggccgcctaaggtcgactcatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgaccttgacgatttaggtccattacataatcaaaattggaatcagatagcacatgaagagtctaacttagctcaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctgctcaaaaattgggacaagttagttaaagaatcatgttcagtaatcaatgcaggtacttcccagtgcattcagaatctcagctatggactgacaggtcgtgggaacctctttacacgatcacgtgaactctctggtgaccgcagggatattgatcttaagacagttgtggcagcatggcatgactcagactggaaaagaataagtgatttttggattatgatcaaattccagatgagacaattaattgttaggcaaacagatcataatgattctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaagaggaactaatagtttcacccaggatgaattaagaactgtgacaactaatttgatatccccattccaagatctgaccccagatcttacggctgaattgctctgtataatgaggctttggggacaccccatgctcactgccagtcaagctgcaggaaaggtacgcgagtctatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgaaataagctatgaattcacacttaagcattggaaggaagtctctcttataaaattcaagaaatgttttgatgcagacgcaggtgaggaactcagtatatttatgaaagataaggcaattagtgccccaaaacaagactggatgagtgtgtttagaagaagcctaatcaaacagcgccatcagcatcatcaggtccccctaccaaatccattcaatcgacggctgttgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaagtcaactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctgtgtcaaaaggcttggacaatgatatctatcgctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgcacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttttgccatcccagttaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccctccaactacagctcttaagaggcacacccaacaagctctgatggaactcagtacgaatccaatgttacgtggcatattctctgacaatgcacaggcagaagaaaataaccttgctaggtttctcctggatagggaggtgatctttccgcgtgtagctcacatcatcattgagcaaaccagtgtcgggaggagaaaacagattcaaggatatttggattcaactagatcgataatgaggaaatcactagaaattaagcccttatccaataggaagcttaatgaaatactggattacaacatcaattacctagcttacaatttggcattactcaagaatgctattgaacctccgacttatttgaaggcaatgacacttgaaacatgtagcatcgacattgcaaggaacctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcttaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaatcttagaaacatggaataatcccccaatcaatagaacattcgaagaatctaccctacatttgcacactggtgcatcatgttgtgtccgacctgtggactcctgcattctctcagaagcattaacagtcaagccacatattacagtaccgtacagcaataaatttgtatttgatgaggacccgctatctgaatatgaaactgcaaaactggaatcgttatcattccaagcccaattaggcaacattgatgctgtagatatgacaggtaaattaacattattgtcccaattcactgcaaggcagattatcaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggcacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgctgcaggtaccttgagcgattaaagctaccacaaggtgatcatttatatattggagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaacgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctgatccagctaatcatgaggtttatttaatttgtatccttgccaacaactttgcattccagactgtctcgcaggcaacaggaatggcgatgactttaactgatcaagggtttactttgatatcacctgaaagaataaatcagtattgggatggtcacttgaagcaagaacgtatcgtagcagaagcaattgataaggtggttctaggagaaaatgctctatttaattcgagtgataatgaattaattctcaaatgtggagggacaccaaatgcacggaatctcatcgatatcgagccagtcgcaactttcatagaatttgaacaattgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattgcacaattaactgcaggctacattaggaaattgattgagggggattgcaatatcgatctaaccagacctatccaaaagcaaatctggaaagcattaggttgtgtagtctattgtcacgatccaatggatcaaagggagtcaacagagtttattgatataaatattaatgaagaaatagaccgcgggatcgatggcgaggaaatctaaacatatcaagaatcagaattagtttaagaaaaaagaagaggattaatcttggttttccccttggtgggtcggcatggcatctccacctcctcgcggtccgacctgggcatccgaaggaggacgcacgtccactcggatggctaagggagcggccggggatccggctgctaacaaagcccgaaaggaagctgagttggctgctgccaccgctgagcaataactagcataaccccttggggcctctaaacgggtcttgaggggttttttgctgaaaggaggaactatatccggatggaatcaccagcttgatttatctccaaaatgattcaaagaaaacaaatcatattaagactatcctaagcacgaacccatatcgtccttcaaatcatggaccccaagggcagcctgagctggagaatcctgctgttcctgagcctggccttcgagctgagctacggcgacgtgcaggtggtggagagcggaggcggagtggtgcagccaggcggcagcctgagactgagctgcgctgcgagtggcttcaccttcagcagcttcgggatgcactgggtgaggcaggcacccggcaagggcctggagtgggtggcctacatcaacaccgacagcaccaccatctactacgcagacagcgtgaagggcaggttcaccatcagcagggacaacagcaagaacaccctgtacctgcagatgaactctttgcgggcagaagacactgccgtgtactactgcgccagtgctggcccctactatggctttgactactggggacagggcaccctggtgaccgtgagcagcgctagcaccaagggcctcagcgtgttccctctggcccccagcagcaagagcaccagcggcggaaccgccgccctgggctgcctggtgaaggactacttccccgagcccgtgaccgtgtcctggaacagcggcgctctgaccagcggagtgcacaccttccctgccgtgctgcagagcagcggcctgtactccctgagcagcgtggtgaccgtgcccagcagcagcctgggcacccagacctacatctgcaacgtgaaccacaagccctccaacaccaaggtggacaagaaggtggagcctaagagctgcgacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacaactccacctaccgcgtggtgagcgtgctgaccgtgctgcaccaggactggctgaacggcaaggagtacaagtgcaaggtgagcaacaaggccctgcccgctcccatcgagaagaccatcagcaaggccaagggccagcctcgggagcctcaggtgtacaccctgccccccagccgcgaagagatgaccaagaaccaggtgagcctgacctgcctggtgaagggcttctacccctccgacatcgccgtggagtgggagagcaacggccagcctgagaacaactacaagaccacccctcccgtgctggacagcgacggcagcttcttcctgtacagcaagctgaccgtggacaagtcccggtggcagcagggcaacgtgttcagctgcagcgtgatgcacgaggccctgcacaaccactacacccagaagagcctgagcctgagccccggacgcgcaaaaagatctagagagggcaggggaagtcttctaacatgcggggacgtggaggaaaatcccgggcctatggagaccgacaccctgctgctctgggtgctgctgctctgggtgcccggctccaccggagacatcgtgatgacccagagtccactgagcctgcccgtgacccccggtgagccagccagcatcagctgcaggagcagcaagagcctggtgcacagcaacggcatcacctacctgtactggtacgtgcagaagcccggacagagcccccagttgctcatttatcagatgagcagcctggcaagcggcgtgcccgacaggttcagcggctccggcagcggcaccgacttcagcctgaagatcagccgggtggaggccgaggacgtgggcgtgtactactgcggccaaattctggagctgcccttcaccttcggccagggaaccaaggtggagatcaagcggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaatgagcattccaccactcacgatctgatctcagtgagaaaaatcaacctgcaactcttggaacaagataagacagtcatccattagtaatttttaagaaaaaaacgataggaccgaacct

SEQ ID NO: 3 - DNA, 12313 bp, synthetic construct
  source 1..12313: mol_type = other DNA
catgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggctaatacgactcactatagggaccaaggggaaaatgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacgctcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattactcacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacacctttcgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgacgaataatccagaatgtaatccggaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaactagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaacaagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctgtcactgggacaactcaggtgcccgctgcagcaacaggtgacacactcgagagttacatggcagcggatcgactgaggcagagatatgctgatgcaggcacccatgatgatgagatgccaccattggaagaggaggaagaggacgacacatctgcaggtccacgcactggaccaactcttgaacaagtggccttggacatccagaacgcagcagttggagctcccatccatacagatgacctgaatgccgcactgggtgatcttgacatctagacaattcagatcccaatctaaaattgacatacctaattgattagttagatggaactacagtggattccataaggttcctgcctaccatcggctttaaagaaaaaaataggcccggacgggttagcaacaagcgactgccggtgccaacagcgcaatccacaatctacaatggatcccactgatctgagcttctccccagatgagatcaataagctcatagagacaggcctgaatactgtagagtattttacttcccaacaagtcacaggaacatcctctcttggaaagaatacaataccaccaggggtcacaggactactaaccaatgctgcagaggcaaagatccaagagtcaactaaccatcagaagggctcagttggtgggggtgcaaaaccaaagaaaccgcgaccaaaaattgccattgtgccagcagatgacaaaacagtgcccggaaagccgatcccaaaccctctattaggtctggactccaccccgagcacccaaactgtgcttgatctaagtgggaaaacattaccatcaggatcctataagggggttaagcttgcgaaatttggaaaagaaaatctgatgacacggttcatcgaggaacccagagagaatcctatcgcaaccagttcccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagacactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagattacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgccaagagaacaataggccagaatggctgggtctcgggagatattactccctgaagtccatctcaattcaccaattgtaaagcataagctatactattacattctacttggaaacctcccaaatgagatcgaccttgacgatttaggtccattacataatcaaaattggaatcagatagcacatgaagagtctaacttagctcaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctgctcaaaaattgggacaagttagttaaagaatcatgttcagtaatcaatgcaggtacttcccagtgcattcagaatctcagctatggactgacaggtcgtgggaacctctttacacgatcacgtgaactctctggtgaccgcagggatattgatcttaagacagttgtggcagcatggcatgactcagactggaaaagaataagtgatttttggattatgatcaaattccagatgagacaattaattgttaggcaaacagatcataatgattctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaagaggaactaatagtttcacccaggatgaattaagaactgtgacaactaatttgatatccccattccaagatctgaccccagatcttacggctgaattgctctgtataatgaggctttggggacaccccatgctcactgccagtcaagctgcaggaaaggtacgcgagtctatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgaaataagctatgaattcacacttaagcattggaaggaagtctctcttataaaattcaagaaatgttttgatgcagacgcaggtgaggaactcagtatatttatgaaagataaggcaattagtgccccaaaacaagactggatgagtgtgtttagaagaagcctaatcaaacagcgccatcagcatcatcaggtccccctaccaaatccattcaatcgacggctgttgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaagtcaactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctgtgtcaaaaggcttggacaatgatatctatcgctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgcacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttttgccatcccagttaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccctccaactacagctcttaagaggcacacccaacaagctctgatggaactcagtacgaatccaatgttacgtggcatattctctgacaatgcacaggcagaagaaaataaccttgctaggtttctcctggatagggaggtgatctttccgcgtgtagctcacatcatcattgagcaaaccagtgtcgggaggagaaaacagattcaaggatatttggattcaactagatcgataatgaggaaatcactagaaattaagcccttatccaataggaagcttaatgaaatactggattacaacatcaattacctagcttacaatttggcattactcaagaatgctattgaacctccgacttatttgaaggcaatgacacttgaaacatgtagcatcgacattgcaaggaacctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcttaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaatcttagaaacatggaataatcccccaatcaatagaacattcgaagaatctaccctacatttgcacactggtgcatcatgttgtgtccgacctgtggactcctgcattctctcagaagcattaacagtcaagccacatattacagtaccgtacagcaataaatttgtatttgatgaggacccgctatctgaatatgaaactgcaaaactggaatcgttatcattccaagcccaattaggcaacattgatgctgtagatatgacaggtaaattaacattattgtcccaattcactgcaaggcagattatcaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggcacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgctgcaggtaccttgagcgattaaagctaccacaaggtgatcatttatatattggagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaacgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctgatccagctaatcatgaggtttatttaatttgtatccttgccaacaactttgcattccagactgtctcgcaggcaacaggaatggcgatgactttaactgatcaagggtttactttgatatcacctgaaagaataaatcagtattgggatggtcacttgaagcaagaacgtatcgtagcagaagcaattgataaggtggttctaggagaaaatgctctatttaattcgagtgataatgaattaattctcaaatgtggagggacaccaaatgcacggaatctcatcgatatcgagccagtcgcaactttcatagaatttgaacaattgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattgcacaattaactgcaggctacattaggaaattgattgagggggattgcaatatcgatctaaccagacctatccaaaagcaaatctggaaagcattaggttgtgtagtctattgtcacgatccaatggatcaaagggagtcaacagagtttattgatataaatattaatgaagaaatagaccgcgggatcgatggcgaggaaatctaaacatatcaagaatcagaattagtttaagaaaaaagaagaggattaatcttggttttccccttggtgggtcggcatggcatctccacctcctcgcggtccgacctgggcatccgaaggaggacgcacgtccactcggatggctaagggagcggccggggatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 4 - DNA, 4567 bp, synthetic construct
  source 1..4567: mol_type = other DNA
catgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggctaatacgactcactatagggaccaaggggaaaaccaagattaatcctcttcttttttcttaaactaattctgattcttgatatgtttagatttcctcgccatcgatcccgcggtctatttctgctcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagactccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcattgggccaggattctcctcgacgtcaccgcatgttagcagacttcctctgccctctcccttgtacagctcgtccatgccgccggtggagtggcggccctcggcgcgttcgtactgttccacgatggtgtagtcctcgttgtgggaggtgatgtccaacttgatgttgacgttgtaggcgccgggcagctgcacgggcttcttggccttgtaggtggtcttgacctcagcgtcgtagtggccgccgtccttcagcttcagcctctgcttgatctcgcccttcagggcgccgtcctcggggtacatccgctcggaggaggcctcccagcccatggtcttcttctgcattacggggccgtcggaggggaagttggtgccgcgcagcttcaccttgtagatgaactcgccgtcctgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaactgaggggacaggatgtcccaggcgaagggcagggggccacccttggtcaccttcagcttggcggtctgggtgccctcgtaggggcggccctcgccctcgccctcgatctcgaactcgtggccgttcacggagccctccatgtgcaccttgaagcgcatgaactccttgatgatggccatgttatcctcctcgcccttgctcaccatcacacaattcgctttatgataacaatctgtgattgtcaccataagcagccacaataaaataaaaggaaacacggacacccaaagtagtcggttccgccacggacttgcgcgttacgacaggccaatcactggtttgtgaccacctgctccgaggttgggattagccgcattcaggggccggaggattcttatgtagctcaataggctcttcacaccttgttcacaactagcgtcccatggcgttagccataggtaggccgccaacgcagcctggaccaccgtcaccggtgagggatgtccagactcatcagcctaagctacactctggggttgagtgctgagcgcaacgcatcgaagattccgaggtggtactgggcttctcgaagtacataagcggataacggatccgtcgctttcaaccacgcaagcagtctatacgacatcaccggggaaacagaagtgcttgttcgtggtggtactggtttgtacccccttctattgaacttggttttgtgcgtctaagttacgggaagggagtataaaacaggcgtacaagggtaccgcaataccggagtactagccgccacgtgggcctctggggtgggtacaaccccagagctgttttaagctaactactctgactcgaggtcggtcacgaaggccataggttccggacctatgtaatctcgagggtcttcgatgatttgagtcaccacttcattttccccttggtgggtcggcatggcatctccacctcctcgcggtccgacctgggcatccgaaggaggacgcacgtccactcggatggctaagggagcggccggggatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 5 - DNA, 5443 bp, synthetic construct
  source 1..5443: mol_type = other DNA
catgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggctaatacgactcactatagggaccaaggggaaaaccaagattaatcctcttcttttttcttaaactaattctgattcttgatatgtttagatttcctcgccatcgatcccgcggtctatttctgctcaggcaccgggcttgcgggtcatgcaccaggtgcgcggtccttcgggcacctcgacgtcggcggtgacggtgaagccgagccgctcgnagaaggggaggttgcggggcgcggaggtctccaggaaggcgggcaccccggcgcgctcggccgcctccactccggggagcacgacggcgctgcccagacccttgccctggtggtcgggcgagactccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcattgggccaggattctcctcgacgtcaccgcatgttagcagacttcctctgccctctcccttgtggtacacgaagttttccattctatttctgttggccaccacgatggtcagcagcttccacaccaccacactcagcagaatgatcaggatcaagcccagagatcccagacaaatggcgatgatgctcagcacggaggtggtggtggcgcttgtgatggcgctcaggtaggtgtcgctttgagccaggtgctgcagggcatcgctcagggacttgttcacggcggccaggttctgagagatatccagagggtcgatgctcaggatctggctgctttccagcttaatggtgctgttgtaggtcacgttggccagctgtgtgatggtgaaccgcaggttatccagctgcagggagacgcacttatacatatcgatcactgtcacgggggagctgctaggctgcaaaatcacagcggctggctgcatgcatttacacagcatgctccggcagttggcgtaaacgatgccgtcgaacagcacgaatctggtcagaaaagagcccaccactgggctgaaggtacatcttgtcaggttgccttgcaggcaggccattgtgtcgtcagacagcacttgggcgtcgttgtatctgcagtacactgtattaggggtgatggtgcactgagaggcaggataagcctggatcaggctgccggtcaccatcactctggttggcagctgggccatcacctcctggttgttgataaaagcgctgattgtagccaggtcgatgatctgggtggcaggctggactgtcagtgtaggcagctcgatcttgatgaccatctgcatgtatgtcaggtccaggcccacgatctgtcctgtcagcaggccggagctcagcagctcggcggcgctgatctgggtgttgaagctcttctcgaccactgtaggcagggtagagccgagcaggattctcagggcctggatggtgatagggctcagggcggggttggtgatctggttgtggaagattgtggtcagttcagtcaggtacaggttcaggatgcttccgatgatggcgtcctgagctttacaattagcagctgtgatggcaggagacaccacgctattgatatgatcctgcacggcctgcacggcggtgcccaggctctgggtagcttgaaccacgtcggccactgcggcgttggttttctggatggcgttcttcaggttcaggatagcagcggcgttctcattggccttgaccagggccacggcggcggtcacctgggcggcggtggccacgccgagagcggccaggccgatcaccactccggcgaaccttctccgccgtcttgtggggatcagctggttccggatggtttccaggttctcgccgatgggctgaagcagctttgtcacggtggcattgtaggagctgatgctggtgatgttgcagccgctgatgggagaatcgatggtaggcatcagcttaaccacgatgaaggcgctagaggcctcggtgtagtacatcagctggcgcacattggtagggatcactccaatctgcatgagagcggcggggtccagagagccggcgccggcgagcaggcagcttaccaccagaaattgtatgattgtgcccatcaaattcgctttatgataacaatctgtgattgtcaccataagcagccacaataaaataaaaggaaacacggacacccaaagtagtcggttccgccacggacttgcgcgttacgacaggccaatcactggtttgtgaccacctgctccgaggttgggattagccgcattcaggggccggaggattcttatgtagctcaataggctcttcacaccttgttcacaactagcgtcccatggcgttagccataggtaggccgccaacgcagcctggaccaccgtcaccggtgagggatgtccagactcatcagcctaagctacactctggggttgagtgctgagcgcaacgcatcgaagattccgaggtggtactgggcttctcgaagtacataagcggataacggatccgtcgctttcaaccacgcaagcagtctatacgacatcaccggggaaacagaagtgcttgttcgtggtggtactggtttgtacccccttctattgaacttggttttgtgcgtctaagttacgggaagggagtataaaacaggcgtacaagggtaccgcaataccggagtactagccgccacgtgggcctctggggtgggtacaaccccagagctgttttaagctaactactctgactcgaggtcggtcacgaaggccataggttccggacctatgtaatctcgagggtcttcgatgatttgagtcaccacttcattttccccttggtgggtcggcatggcatctccacctcctcgcggtccgacctgggcatccgaaggaggacgcacgtccactcggatggctaagggagcggccggggatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 6 - DNA, 7615 bp, synthetic construct
  source 1..7615: mol_type = other DNA
catgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggctaatacgactcactatagggaccaaggggaaaaccaagattaatcctcttcttttttcttaaactaattctgattcttgatatgtttagatttcctcgccatcgatcccgcggtctatttctgcgctcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagactccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcattgggccaggattctcctcgacgtcaccgcatgttagcagacttcctctgccctctccggtgctatccaggcccagcagcgggttcggaatcggtttgccactggtcatacagcaaagcataattgtcaccattactatggcaatcaagccagctataaaacctagccaaatgtaccatggccattttatatactgctcatactttccaagttcttggagatcgatgagagattcatttaaattcttggcaacctcattgaggcggtcaatttctttttgaatgtttacaactgaagcattaatgccagagatgtcacctaaatcaacatctggtgatgtatgattcttaaaatatttatctaactcctccttgaatgagtctaattcaggttgcaaaggatcataaactgtgttgttgacaattcctattacaacatcacagttaccagacacaaatgtgttgtctgtagtaatgatttgtggttcataaaaattcctttgtgttacaaaccagtgtgtgccatttgaaacaaagacaccttcacgaggaaagtgtgcttttccatcatgacaaatggcaggagcagttgtgaagttcttttcttgtgcagggacataagtcacatgcaagaagactacaccatgaggtgctgactgagggaaggacataagatgatagccctttccacaaaaatcaactctttttgattgtccaagtacacactctgacattttagtagcagcaagattagcagaagctctgatttctgcagctctaattaattgttgagtcacatatgtctgcaaactttgaagtctgcctgtgatcaacctatcaatttgcacttcagcctcaactttgtcaagacgtgaaaggatatcatttaaaacacttgaaattgcaccaaaattggagctaagttgtttaacaagcgtgtttaaagcttgtgcattttggttgaccacatcttgaagttttccaagtgcacttgctgtggaagaaagtgagtcttgaattttgccaatagcactattaaattggttggcaatcaatttttggttctcatagagaacattctgtgtaactccaataccattaaacctataagccatttgcatagcaaatggtatttgtaatgcagcacctgcaccaaaggtccaaccagaagtgattgtacccgctaacagtgcagaagtgtattgagcaatcatttcatctgtgagcaaaggtggcaaaacagtaaggccgttaaacttttgtgcacaaatgaggtctctagcagcaatatcaccaaggcaatcaccatattgtttgatgaagccagcatctgcaagtgtcactttgttgaaaagtagatcttcaataaatgacctcttgcttggttttgatggatctggtaatatttgtgaaaaattaaaaccaccaaaatctttaattggtggtgttttgtaaatttgtttgacttgtgcaaaaacttcttgggtgtttttgtcttgttcaacagctattccagttaaagcacggtttaattgtgtacaaaaactgccatattgcaacaaaagattgctgcattcagttgaatcaccacaaatgtacattgtacaatctactgatgtcttggtcatagacactggtagaatttctgtggtaacactaatagtaaaatttgtgggtatggcaatagagttattagagtaagcaactgaattttctgcaccaagtgacatagtgtaggcaatgatggattgactagctacactacgtgcccgccgaggagaattagtctgagtctgataactagcgcatatacctgcaccaatgggtatgtcacactcatatgagttgttgacatgttcagcccctattaaacagcctgcacgtgtttgaaaaacattagaacctgtagaataaacacgccaagtaggagtaagttgatctgcatgaatagcaacagggacttctgtgcagttaacatcctgataaagaacagcaacctggttagaagtatttgttcctggtgttataacactgacaccaccaaaagaacatggtgtaatgtcaagaatctcaagtgtctgtggatcacggacagcatcagtagtgtcagcaatgtctctgccaaattgttggaaaggcagaaactttttgttagactcagtaagaacacctgtgcctgttaaaccattgaagttgaaattgacacatttgtttttaaccaaattagtagactttttaggtccacaaacagttgctggtgcatgtagaagttcaaaagaaagtactactactctgtatggttggtaaccaacaccattagtgggttggaaaccatatgattgtaaaggaaagtaacaattaaaaccttcaacaccattacaaggtgtgctaccggcctgatagatttcagttgaaatatctctctcaaaaggtttgagattagacttcctaaacaatctatacaggtaattataattaccaccaaccttagaatcaagattgttagagttccaagctataacgcagcctgtaaaatcatctggtaatttataattataatcagcaatctttccagtttgccctggagcgatttgtctgacttcatcacctctaattacaaatgaatctgcatagacattagtaaagcagagatcatttaatttagtaggagacactccataacacttaaaagtggaaaatgatgcggaattatataggacagaataatcagcaacacagttgctgattctcttcctgttccaagcataaacagatgcaaatctggtggcgttaaaaacttcaccaaaagggcacaagtttgtaatattaggaaatctaacaatagattctgttggttggactctaaagttagaagtttgatagattcctttttctacagtgaaggatttcaacgtacactttgtttctgagagagggtcaagtgcacagtctacagcatctgtaatggttccattttcattatattttaatagaaaagtcctaggttgaagataacccacataataagctgcagcaccagctgtccaacctgaagaagaatcaccaggagtcaaataacttctatgtaaagcaagtaaagtttgaaacctagtgatgttaatacctattggcaaatctaccaatggttctaaagccgaaaaaccctgagggagatcacgcactaaattaataggcgtgtgcttagaatatattttaaaataaccatcaatattcttaaacacaaattccctaagatttttgaaattaccctgttttccttcaaggtccataagaaaaggctgagagacatattcaaaagtgcaattattcgcactagaataaactctgaactcactttccatccaacttttgttgtttttgtggtaataaacacccaaaaatggatcattacaaaattgaaattcacagactttaataacaacattagtagcgttattaacaataagtagggactgggtcttcgaatctaaagtagtaccaaaaatccagcctcttattatgttagacttctcagtggaagcaaaataaacaccatcattaaatggtaggacagggttatcaaacctcttagtaccattggtcccagagacatgtatagcatggaaccaagtaacattggaaaagaaaggtaagaacaagtcctgagttgaatgtaaaactgaggatctgaaaactttgtcagggtaataaacaccacgtgtgaaagaattagtgtatgcagggggtaattgagttctggttgtaagattaacacactgactagagactagcggcaataaaacaagaaaaacaaacataattcgctttatgataacaatctgtgattgtcaccataagcagccacaataaaataaaaggaaacacggacacccaaagtagtcggttccgccacggacttgcgcgttacgacaggccaatcactggtttgtgaccacctgctccgaggttgggattagccgcattcaggggccggaggattcttatgtagctcaataggctcttcacaccttgttcacaactagcgtcccatggcgttagccataggtaggccgccaacgcagcctggaccaccgtcaccggtgagggatgtccagactcatcagcctaagctacactctggggttgagtgctgagcgcaacgcatcgaagattccgaggtggtactgggcttctcgaagtacataagcggataacggatccgtcgctttcaaccacgcaagcagtctatacgacatcaccggggaaacagaagtgcttgttcgtggtggtactggtttgtacccccttctattgaacttggttttgtgcgtctaagttacgggaagggagtataaaacaggcgtacaagggtaccgcaataccggagtactagccgccacgtgggcctctggggtgggtacaaccccagagctgttttaagctaactactctgactcgaggtcggtcacgaaggccataggttccggacctatgtaatctcgagggtcttcgatgatttgagtcaccacttcattttccccttggtgggtcggcatggcatctccacctcctcgcggtccgacctgggcatccgaaggaggacgcacgtccactcggatggctaagggagcggccggggatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 7 - DNA, 4567 bp, synthetic construct; identical to SEQ ID NO: 4
  source 1..4567: mol_type = other DNA

SEQ ID NO: 8 - DNA, 5485 bp, synthetic construct
  source 1..5485: mol_type = other DNA
catgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggctaatacgactcactatagggaccaaggggaaaaccaagattaatcctcttcttttttcttaaactaattctgattcttgatatgtttagatttcctcgccatcgatcccgcggtctatttctggggcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccattgggccaggattctcctcgacgtcaccgcatgttagcagacttcctctgccctctcccttgtacagctcgtccatgccgccggtggagtggcggccctcggcgcgttcgtactgttccacgatggtgtagtcctcgttgtgggaggtgatgtccaacttgatgttgacgttgtaggcgccgggcagctgcacgggcttcttggccttgtaggtggtcttgacctcagcgtcgtagtggccgccgtccttcagcttcagcctctgcttgatctcgcccttcagggcgccgtcctcggggtacatccgctcggaggaggcctcccagcccatggtcttcttctgcattacggggccgtcggaggggaagttggtgccgcgcagcttcaccttgtagatgaactcgccgtcctgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaactgaggggacaggatgtcccaggcgaagggcagggggccacccttggtcaccttcagcttggcggtctgggtgccctcgtaggggcggccctcgccctcgccctcgatctcgaactcgtggccgttcacggagccctccatgtgcaccttgaagcgcatgaactccttgatgatggccatgttatcctcctcgcccttgctcaccattgtagattgtggattgcgctgttggcaccggcagtcgcttgttgctaacccgtccgggcctatttttttctttaaagccgatggtaggcaggaaccttatggaatccactgtagttccatctaactaatcaattaggtatgtcaattttagattgggatctgaattgtctagatgtcaagatcacccagtgcggcattcaggtcatctgtatggatgggagctccaactgctgcgttctggatgtccaaggccacttgttcaagagttggtccagtgcgtggacctgcagatgtgtcgtcctcttcctcctcttccaatggtggcatctcatcatcatgggtgcctgcatcagcatatctctgcctcagtcgatccgctgccatgtaactctcgagtgtgtcacctgttgctgcagcgggcacctgagttgtcccagtgacagatgagaacgggttgactcctcccctggtgtctgcccctcgatttgctgtggtcaatttggcaagtgtatttgccatctcggtgcgttcggcttgagttagaccgagatcttctgccatcctcatgtcaactgcaccctgttgttttcttgcagtttccattcccaattggaaatatgtcttgttcatgtatgatctggagaaagcgtagttcctcatgttgacatctaacacatagcctattcccatagcatagctatatagcagtgggtagtttgctgcagcaaaatccatcaaatgtggtgactccaatagggccaaatatcgggcctgctcaccaagggtctggtataatgccatgagggactttagctttgttagctctcctgaaaatgcagctaaagcaagtgtgggccatctagttcctaatgcatattttagtgtcaaaaagaagcctcccattccacaattctctatatactttccaacatcccctaccatagcataatacctatttgatacaaggctttgtgctcgggcaagctgcagttcaaaggtgaggaaatgtctgaccaccattcccttccggattacattctggattattcgtcgagcctccggttgcaggagatatctcgggttgatcctgccttgctgacgatatttttgcaggcgtttctcaatagaagcagcaggttggtctggcgcagtcatgcacttgcaagtcactatccatgcctgcattaggacactgtaacacatatctaagaaagtctcaatctcatcccatgcagttccctcgacttcggaatcaacgaaaggtgttgcatggtttagtgtgtcaggtagatcttctgcaagtgcagcataggcattgatctctccacggctcatacctgaacgagcattggggattaagcggaatgatccctcctcaaagccatcgatctctaccctttcgatatcagcttctggtgactggtcagctaatttgacatgattgagcattgtggctgatggtagcgaaaacattgtgagtaatgctccaaagcgatgggaatcccttgcaccattactgagaacaatccgtaggcagaatagaagaagccgggatcttagctctgggttattagaggttagtataaataccctgattaccggttttagtgttgtaggtgggattgtaccttcctcactctgatcttgcagttcttgagtgagcgtgaatcgctcatatgctttaagcacggatgacatttttgtgtcggggacgaaaaattgcccaaacttgataggtccttattgaactactctgactcgaggtcggtcacgaaggccataggttccggacctatgtaatctcgagggtcttcgatgatttgagtcaccacttcattttccccttggtgggtcggcatggcatctccacctcctcgcggtccgacctgggcatccgaaggaggacgcacgtccactcggatggctaagggagcggccggggatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 9 - DNA, 41 bp, synthetic construct
  source 1..41: mol_type = other DNA
cctcctcgcccttgctcaccattgtagattgtggattgcgc

SEQ ID NO: 10 - DNA, 46 bp, synthetic construct
  source 1..46: mol_type = other DNA
ccgacctcgagtcagagtagttcaataaggacctatcaagtttggg

SEQ ID NO: 11 - DNA, 21 bp, synthetic construct
  source 1..21: mol_type = other DNA
atggtgagcaagggcgaggag

SEQ ID NO: 12 - DNA, 21 bp, synthetic construct
  source 1..21: mol_type = other DNA
actactctgactcgaggtcgg

SEQ ID NO: 13 - DNA, 46 bp, synthetic construct
  source 1..46: mol_type = other DNA
cccaaacttgataggtccttattgaactactctgactcgaggtcgg

SEQ ID NO: 14 - DNA, 41 bp, synthetic construct
  source 1..41: mol_type = other DNA
gcgcaatccacaatctacaatggtgagcaagggcgaggagg